(19) United States
(12) Patent Application Publication
(10) Pub. No.: US 2024/0174724 A1
(43) Pub. Date: May 30, 2024

(54) ARENAVIRUSES USED IN TREATMENTS OF PROSTATE CANCER

(71) Applicant: Hookipa Biotech GmbH, Vienna (AT)

(72) Inventors: Igor Matushansky, Brooklyn, NY (US); Andy Hwang, Jersey City, NJ (US); Kianoosh Katchar, Hoboken, NJ (US); Henning Lauterbach, Eching (DE); Klaus Orlinger, Vienna (AT); Sarah Schmidt, Vienna (AT); Felix Stemeseder, Vienna (AT)

(21) Appl. No.: 18/283,369

(22) PCT Filed: Mar. 22, 2022
(86) PCT No.: PCT/EP2022/057532

§ 371 Date: Sep. 21, 2023

Related U.S. Application Data
(60) Provisional application No. 63/165,028, filed on Mar. 23, 2021.

(51) Int. Cl. C07K 14/47; A61K 39/00; A61P 37/04; C12N 7/00; C12N 9/16

(52) U.S. Cl. CPC C07K 14/4748; A61K 39/001193; A61K 39/001194; A61K 39/001195; A61P 37/04; C12N 7/00; C12N 9/16; C12Y 301/03002; A61K 2039/5256; A61K 2039/55511; C12N 2760/10034; C12N 2760/10043

(57) ABSTRACT

The present application relates to arenaviruses expressing prostate cancer-related antigens. In particular, described herein is a modified arenavirus particle which is engineered to carry a heterologous open reading frame (ORF) encoding a prostate cancer-related antigen or an antigenic fragment thereof, wherein the prostate cancer-related antigen is selected from the group consisting of: prostatic acid phosphatase (PAP), prostate specific antigen (PSA), and prostate-specific membrane antigen (PSMA). Also described herein are arenavius genome segments or S segments encoding the prostate cancer-related antigen or an antigenic fragment thereof, cDNA, DNA expression vector, a pharmaceutical composition comprising such an arenavirus particle, methods of generating such an arenavirus particle and treating prostate cancer using such an arenavirus particle, and a kit for such usage.

### CROSS-REFERENCE TO RELATED APPLICATIONS

This application claims the benefit of U.S. Ser. No. 63/165,028, filed Mar. 23, 2021, which is herein incorporated by reference in its entirety.

### REFERENCE TO SEQUENCE LISTING SUBMITTED ELECTRONICALLY

This application contains a sequence listing, which is submitted electronically as an ASCII formatted sequence listing with a file name “13194-062-228_SEQ_LISTING” and a creation date of Mar. 21, 2022 and having a size of 56,767 bytes. The sequence listing submitted is part of the specification and is herein incorporated by reference in its entirety.

### 1. INTRODUCTION

The present application relates to arenaviruses expressing prostate cancer-related antigens. In particular, described herein is a modified arenavirus particle which is engineered to carry a heterologous open reading frame (ORF) encoding a prostate cancer-related antigen or an antigenic fragment thereof, wherein the prostate cancer-related antigen is selected from the group consisting of: prostatic acid phosphatase (PAP), prostate specific antigen (PSA), and prostate-specific membrane antigen (PSMA). Also described herein are arenavirus genome segments, such as S segments, encoding the prostate cancer-related antigen or an antigenic fragment thereof, cDNA, DNA expression vector, a pharmaceutical composition comprising such an arenavirus particle, methods of generating such an arenavirus particle and treating prostate cancer using such an arenavirus particle, and a kit for such usage.

### 2. BACKGROUND

### 2.1 Prostate Cancer

Prostate cancer is an androgen-dependent type of cancer and is among the most common cancers in men worldwide. It is also the most common cancer besides skin cancer affecting men in the United States (U.S.). It is estimated that there are about 191,930 new cases of prostate cancer and about 33,330 deaths from prostate cancer in the U.S. in 2020. In the European Union (EU), prostate cancer is ranked first among the most frequently diagnosed cancer among men, with around 345,000 new cases estimated in 2012. Prostate cancer accounted for 24% of all new cancers in the same year in the EU. For 2015 the estimated number of new prostate cancer cases was about 365,000 in the EU. About 1 out of 9 men will be diagnosed with prostate cancer during their lifetime. Age is the leading risk factor in prostate cancer, with 60% of the disease diagnosed in men over 65 years of age and rarely found in men under 40 years old. Following lung cancer, prostate cancer is the second leading cause of cancer death in US males, accounting for 10% of all cancer deaths in 2020 (Siegel, Miller, and Jemal, 2020, CA Cancer J Clin, 70: 7-30). In the EU, a total of 78,800 men are predicted to die from prostate cancer in 2020 (Carioli et al., 2020, Ann Oncol, 31: 650-58.).

For localized prostate cancer, surgery, radiation, and androgen deprivation therapy (ADT) achieved with surgical castration or medical orchiectomy are often curative. Non-standard ablative techniques, such as cryotherapy, high-intensity ultrasound, and photodynamic therapy are also used, however, long-term data are still lacking. Patients with lymph node metastases are often treated with definitive radiotherapy (RT) and ADT with or without radical prostatectomy (Bekelman et al., 2018, J Clin Oncol, 36: 3251-58; NCCN_Guidelines® 2019, NCCN Clinical Practice Guidelines in Oncology). The 5-year survival rate for patients with localized or regional prostate cancer is almost 100%.

About 15% of prostate cancer patients present with metastatic disease upon diagnosis. In addition, about 20% of patients with clinically localized disease will fail local therapy and develop incurable metastatic disease (Cooperberg et al., 2004, J Urol, 171: 1393-401). While ADT remains the standard treatment for patients with metastatic prostate cancer, progression usually occurs within 1-2 years after initial response with a 5-year survival rate of 31%. Prostate cancer that has progressed despite castrate levels of androgens (<50 ng/mL) is known as castration-resistant prostate cancer (CRPC). Estimated survival of patients who developed metastatic CRPC (mCRPC) is 2-3 years (Scher et al., 2016, J Clin Oncol, 34: 1402-18).

Treatment options for metastatic prostate cancer remain limited. Antiandrogen agents and cytotoxic chemotherapy are among the top lines of treatment options. Standard therapies that have demonstrated survival and quality-of-life benefits include abiraterone acetate/prednisone, enzalutamide, radium-223, and docetaxel/prednisone. Therapies that demonstrated survival benefit with unclear quality-of-life benefit include sipuleucel-T and cabazitaxel/prednisone (Basch et al., 2014, J Clin Oncol, 32: 3436-48). Despite of the available treatment options for prostate cancer, the demand for new treatments is still high, especially for patients with poor prognosis or in advanced stages. For example, almost all patients will eventually develop resistance to the new-generation antiandrogen agents including enzalutamide due to various mechanisms (Watson, Arora, and Sawyers, 2015, Nat Rev Cancer, 15: 701-11). There has been increasing interests in developing new therapies in prostate cancer to overcome drug resistance. The compositions and methods described herein address this need and provide related advantages.

### 2.2 Prostate Cancer-Related Antigens

Prostate cancer-related antigens encompass PAP, PSA, and PSMA, which have been most commonly used as target antigens in immunotherapies for prostate cancer (Karan, 2013, Immunotherapy, 5: 907-10).

PAP is a glycoprotein with the molecular weight of 100 kDa secreted by prostate epithelial cells (Vihko, Kontturi, and Korhonen, 1978, Clin Chem, 24: 466-70.). There are two forms of PAP. A cellular form (cPAP) is highly expressed in the prostate cells and a secretory form (sPAP) is mostly released into seminal fluid (Lin et al., 2001, J Urol, 166: 1943-50.). PAP is mainly restricted to benign and malignant prostate tissue with low expression in other tissues. Its expression is highest in tumors with high Gleason scores (6 and 7), but it is also found in other adenocarcinomas such as gastric, breast and colon (Kiessling A, et al. Cancers. 2012; 4:193-217.). PAP-specific immune activation is correlated with survival of patients with CRPC treated with sipuleucel-T (Sheikh et al., 2013, Cancer Immunol Immunother, 62: 137-47).

PSA is a kallikrein-related peptidase that is almost exclusively secreted by prostate epithelial cells and is the diagnostic biomarker for diagnosis and monitoring of prostate cancer (Kiessling A, et al. Cancers. 2012; 4:193-217). PSA can be detected in the majority of prostate cancer tissues. PSA has been extensively explored as a target antigen by multiple immunotherapy platforms, including adenovirus, poxviruses, listeria, plasmid DNA, peptide, and dendritic cells (DCs), at various stages of clinical development, most of which are in early phases (Venturini and Drake, 2019, Cold Spring Harb Perspect Med, 9). Both preclinical and clinical studies have demonstrated that PSA, as an immunotherapy target, induces generation of PSA-specific CD8+ T cells (Karan et al., 2011, Immunotherapy, 3: 735-46; Lubaroff et al., 2009, Clin Cancer Res, 15: 7375-80).

PSMA is a transmembrane glycoprotein expressed within the prostate tissue and its expression level increases after androgen ablation (Wright et al., 1996, Urology, 48: 326-34). PSMA is a marker for normal prostate cells and can be detected in most prostate cancer tumors, particularly undifferentiated, metastatic CRPC. Though PSMA can be found in other normal tissues, it has 100 to 1000 folds lower expression compared to prostate tissue (Kiessling A, et al. Cancers. 2012; 4:193-217). Antigen-specific CD4+ and CD8+ T cell responses have been reported with cancer vaccine targeting PSMA (Chudley et al., 2012, Cancer Immunol Immunother, 61: 2161-70).

### 3. SUMMARY

### 3.1 Arenavirus Genome Segments

In one aspect, provided herein is an arenavirus S segment. In some embodiments, the arenavirus S segment is engineered to carry an open reading frame (ORF) encoding a prostate cancer-related antigen or an antigenic fragment thereof. In some specific embodiments, the prostate cancer-related antigen is selected from the group consisting of: prostatic acid phosphatase (PAP), prostate specific antigen (PSA), and prostate-specific membrane antigen (PSMA).

In some embodiments, the arenavirus S segment is engineered to carry a heterologous ORF encoding PAP or an antigenic fragment thereof in a position under control of an arenavirus 5′ UTR, and an ORF encoding arenaviral glycoprotein (GP) in a position under control of an arenavirus 3′ UTR. In other embodiments, the arenavirus S segment is engineered to carry a heterologous ORF encoding PAP or an antigenic fragment thereof in a position under control of an arenavirus 3′ UTR, and an ORF encoding GP in a position under control of an arenavirus 5′ UTR. In other embodiments, the arenavirus S segment is engineered to carry a heterologous ORF encoding PAP or an antigenic fragment thereof in a position under control of an arenavirus 5′ UTR, and an ORF encoding arenaviral nucleoprotein (NP) in a position under control of an arenavirus 3′ UTR. In other embodiments, the arenavirus S segment is engineered to carry a heterologous ORF encoding PAP or an antigenic fragment thereof in a position under control of an arenavirus 3′ UTR, and an ORF encoding NP in a position under control of an arenavirus 5′ UTR. In some specific embodiments, the amino acid sequence of the PAP or an antigenic fragment thereof comprises at least 80% or 90% identity to SEQ ID NO: 1. In other specific embodiments, the amino acid sequence of the PAP or an antigenic fragment thereof comprises SEQ ID NO: 1. In yet other specific embodiments, the amino acid sequence of the PAP or an antigenic fragment thereof consists of SEQ ID NO: 1. In other specific embodiments, the amino acid sequence of the PAP or an antigenic fragment thereof comprises SEQ ID NO: 18. In yet other specific embodiments, the amino acid sequence of the PAP or an antigenic fragment thereof consists of SEQ ID NO: 18. In some specific embodiments, the nucleotide sequence of the ORF encoding the PAP or an antigenic fragment thereof comprises at least 50%, 60%, 70%, 80%, or 90% identity to SEQ ID NO: 5. In other specific embodiments, the nucleotide sequence of the ORF encoding the PAP or an antigenic fragment thereof comprises SEQ ID NO: 5. In yet other specific embodiments, the nucleotide sequence of the ORF encoding the PAP or an antigenic fragment thereof consists of SEQ ID NO: 5.

In some embodiments, the arenavirus S segment is engineered to carry a heterologous ORF encoding PSA or an antigenic fragment thereof in a position under control of an arenavirus 5′ UTR, and an ORF encoding arenaviral glycoprotein (GP) in a position under control of an arenavirus 3′ UTR. In other embodiments, the arenavirus S segment is engineered to carry a heterologous ORF encoding PSA or an antigenic fragment thereof in a position under control of an arenavirus 3′ UTR, and an ORF encoding GP in a position under control of an arenavirus 5′ UTR. In other embodiments, the arenavirus S segment is engineered to carry a heterologous ORF encoding PSA or an antigenic fragment thereof in a position under control of an arenavirus 5′ UTR, and an ORF encoding arenaviral nucleoprotein (NP) in a position under control of an arenavirus 3′ UTR. In other embodiments, the arenavirus S segment is engineered to carry a heterologous ORF encoding PSA or an antigenic fragment thereof in a position under control of an arenavirus 3′ UTR, and an ORF encoding NP in a position under control of an arenavirus 5′ UTR. In some specific embodiments, the amino acid sequence of the PSA or an antigenic fragment thereof comprises at least 80% or 90% identity to SEQ ID NO: 2. In other specific embodiments, the amino acid sequence of the PSA or an antigenic fragment thereof comprises SEQ ID NO: 2. In yet other specific embodiments, the amino acid sequence of the PSA or an antigenic fragment thereof consists of SEQ ID NO: 2. In some specific embodiments, the nucleotide sequence of the ORF encoding the PSA or an antigenic fragment thereof comprises at least 50%, 60%, 70%, 80%, or 90% identity to SEQ ID NO: 6. In other specific embodiments, the nucleotide sequence of the ORF encoding the PSA or an antigenic fragment thereof comprises SEQ ID NO: 6. In yet other specific embodiments, the nucleotide sequence of the ORF encoding the PSA or an antigenic fragment thereof consists of SEQ ID NO: 6.

In some embodiments, the arenavirus S segment is engineered to carry a heterologous ORF encoding an antigenic fragment of PSMA in a position under control of an arenavirus 5′ UTR, and an ORF encoding arenaviral glycoprotein (GP) in a position under control of an arenavirus 3′ UTR. In other embodiments, the arenavirus S segment is engineered to carry a heterologous ORF encoding an antigenic fragment of PSMA in a position under control of an arenavirus 3′ UTR, and an ORF encoding GP in a position under control of an arenavirus 5′ UTR. In other embodiments, the arenavirus S segment is engineered to carry a heterologous ORF encoding an antigenic fragment of PSMA in a position under control of an arenavirus 5′ UTR, and an ORF encoding arenaviral nucleoprotein (NP) in a position under control of an arenavirus 3′ UTR. In other embodiments, the arenavirus S segment is engineered to carry a heterologous ORF encoding an antigenic fragment of PSMA in a position under control of an arenavirus 3′ UTR, and an ORF encoding NP in a position under control of an arenavirus 5′ UTR. In some specific embodiments, the amino acid sequence of the antigenic fragment of PSMA comprises at least 80% or 90% identity to SEQ ID NO: 3 or SEQ ID NO: 4. In other specific embodiments, the amino acid sequence of the antigenic fragment of PSMA comprises SEQ ID NO: 3 or SEQ ID NO: 4. In yet other specific embodiments, the amino acid sequence of the antigenic fragment of PSMA consists of SEQ ID NO: 3 or SEQ ID NO: 4. In some specific embodiments, the nucleotide sequence of the ORF encoding the antigenic fragment of PSMA comprises at least 50%, 60%, 70%, 80%, or 90% identity to SEQ ID NO: 7 or SEQ ID NO: 8. In other specific embodiments, the nucleotide sequence of the ORF encoding the antigenic fragment of PSMA comprises SEQ ID NO: 7 or SEQ ID NO: 8. In yet other specific embodiments, the nucleotide sequence of the ORF encoding the antigenic fragment of PSMA consists of SEQ ID NO: 7 or SEQ ID NO: 8.

In some embodiments, the invention further comprises a cDNA encoding the above-identified arenavirus S segments. In other embodiments, the invention further comprises a DNA expression vector comprising the cDNA. In other embodiments, the invention further comprises a host cell comprising the above-identified arenavirus S segments, cDNA or the DNA expression vector.

### 3.2 Arenavirus Particles and Methods of Generation Such Arenavirus Particles

In another aspect, provided herein is a tri-segmented arenavirus particle comprising one arenavirus L segment and two arenavirus S segments. In some embodiments, the two arenavirus S segments are engineered to carry an open reading frame (ORF) encoding a prostate cancer-related antigen or an antigenic fragment thereof as described herein. In some embodiments, one of the two arenavirus S segments includes the ORF encoding the arenavirus GP and the other includes the ORF encoding the arenavirus NP.

In some embodiments, the tri-segmented arenavirus particle has stable expression of the prostate cancer-related antigen(s) or antigenic fragment(s) thereof after being passaged at least 4, 5, 6, 7, 8, 9, or 10 generations.

In a specific embodiment, a tri-segmented arenavirus particle comprises one arenavirus L segment and two arenavirus S segments, wherein a first arenavirus S segment is engineered to carry a heterologous ORF consisting of SEQ ID NO: 5 in a position under control of an arenavirus 5′ UTR and an ORF encoding arenaviral nucleoprotein (NP) in a position under control of an arenavirus 3′ UTR, and a second arenavirus S segment is engineered to carry a heterologous ORF consisting of SEQ ID NO: 6 in a position under control of an arenavirus 5′ UTR and an ORF encoding arenaviral glycoprotein (GP) in a position under control of an arenavirus 3′ UTR.

In a specific embodiment, a tri-segmented arenavirus particle comprises one arenavirus L segment and two arenavirus S segments, wherein a first arenavirus S segment is engineered to carry a heterologous ORF consisting of SEQ ID NO: 8 in a position under control of an arenavirus 5′ UTR and an ORF encoding arenaviral nucleoprotein (NP) in a position under control of an arenavirus 3′ UTR, and a second arenavirus S segment is engineered to carry a heterologous ORF consisting of SEQ ID NO: 7 in a position under control of an arenavirus 5′ UTR and an ORF encoding arenaviral glycoprotein (GP) in a position under control of an arenavirus 3′ UTR.

In a specific embodiment, a tri-segmented arenavirus particle comprises one arenavirus L segment and two arenavirus S segments, wherein a first arenavirus S segment is engineered to carry a heterologous ORF consisting of SEQ ID NO: 7 in a position under control of an arenavirus 5′ UTR and an ORF encoding arenaviral nucleoprotein (NP) in a position under control of an arenavirus 3′ UTR, and a second arenavirus S segment is engineered to carry a heterologous ORF consisting of SEQ ID NO: 8 in a position under control of an arenavirus 5′ UTR and an ORF encoding arenaviral glycoprotein (GP) in a position under control of an arenavirus 3′ UTR.

In some embodiments, the tri-segmented arenavirus particle is derived from lymphocytic choriomeningitis virus (LCMV) or Pichinde virus (PICV). In some specific embodiments, the LCMV is MP strain, WE strain, Armstrong strain, Armstrong Clone 13 strain, or LCMV clone 13 expressing the glycoprotein of LCMV strain WE instead of endogenous LCMV clone 13 glycoprotein. In other specific embodiments, the PICV is strain Munchique CoAn4763 isolate P18, or P2 strain.

In a specific embodiment, a tri-segmented arenavirus particle comprises two S segments, wherein one of the two S segments comprises SEQ ID NO. 10, and the other one of the two S segments comprises SEQ ID NO. 11.

In a specific embodiment, a tri-segmented arenavirus particle comprises two S segments, wherein one of the two S segments comprises SEQ ID NO. 12, and the other one of the two S segments comprises SEQ ID NO. 13.

In a specific embodiment, a tri-segmented arenavirus particle comprises two S segments, wherein one of the two S segments comprises SEQ ID NO. 14, and the other one of the two S segments comprises SEQ ID NO. 15.

In a specific embodiment, a tri-segmented arenavirus particle comprises two S segments, wherein one of the two S segments comprises SEQ ID NO. 16, and the other one of the two S segments comprises SEQ ID NO. 17.

In some embodiments, the tri-segmented arenavirus particle is infectious and replication competent. In other embodiments, the tri-segmented arenavirus particle is attenuated.

In another aspect, provided herein is a method of generating a tri-segmented arenavirus particle, wherein the method comprises: (i) transfecting into a host cell the nucleic acids of two arenavirus S segments and one arenavirus L segment, wherein the two arenavirus S segments are engineered to carry an open reading frame (ORF) encoding a prostate cancer-related antigen or an antigenic fragment thereof as described herein; (ii) maintaining the host cell under conditions suitable for virus formation; and
(iii) harvesting the cell culture supernatant containing the arenavirus particle. In some specific embodiments, the nucleic acids are cDNA. In other specific embodiments, the nucleic acids are RNA.

In some embodiments, the transcription of the arenavirus L segment and the two arenavirus S segments are performed using a bidirectional expression cassette. In some embodiments, the method further comprises transfecting one or more nucleic acids encoding an arenavirus polymerase into the host cell. In some specific embodiments, the arenavirus polymerase is the arenavirus L protein. In other embodiments, the method further comprises transfecting one or more nucleic acids encoding the arenavirus NP protein into the host cell. In some embodiments, transcription of the arenavirus L segment and the two arenavirus S segments are each under the control of a promoter. In some specific embodiments, the promoter is selected from the group consisting of: (i) a RNA polymerase I promoter; (ii) a RNA polymerase II promoter; and (iii) a T7 promoter.

### 3.3 Methods of Treatment and Kits Thereof

In another aspect, provided herein is a pharmaceutical composition comprising an arenavirus particle as identified above, and a pharmaceutically acceptable carrier.

In another aspect, provided herein is a method for treating prostate cancer comprising administering to a subject in need thereof the pharmaceutical composition in a therapeutically effective amount.

In another aspect, provided herein is a method for treating prostate cancer in a subject in need thereof, wherein the method comprises (i) administering to the subject a first pharmaceutical composition, wherein the first pharmaceutical composition comprises one or more arenavirus particles as identified above; and (ii) administering to the subject, after a period of time, a second pharmaceutical composition, wherein the second pharmaceutical composition comprises one or more arenavirus particles as identified above. In some embodiments, the method further comprises repeating (i) and (ii). In some embodiments, the one or more arenavirus particles from the first pharmaceutical composition are derived from different arenavirus species but carry ORF(s) encoding the same prostate cancer-related antigens or antigenic fragments thereof, when compared to the one or more arenavirus particles from the second pharmaceutical composition. In some specific embodiments, the one or more arenavirus particles from the first pharmaceutical composition are derived from PICV, and the one or more arenavirus particles from the second pharmaceutical composition are derived from LCMV. In other specific embodiments, the one or more arenavirus particles from the first pharmaceutical composition are derived from LCMV, and the one or more arenavirus particles from the second pharmaceutical composition are derived from PICV.

In some embodiments, the first and the second pharmaceutical compositions are administered intravenously. In other embodiments, the first and the second pharmaceutical compositions are administered intratumorally. In yet other embodiments, the first pharmaceutical composition is administered intratumorally, and the second pharmaceutical composition is administered intravenously. In still yet other embodiments, the first pharmaceutical composition is administered intravenously, and the second pharmaceutical composition is administered intratumorally.

In some embodiments, a second agent is administered in combination with the first and/or the second pharmaceutical composition. In some specific embodiments, the second agent is an agent to treat prostate cancer. In certain embodiments, the second agent is selected from the group consisting of docetaxel, mitoxantrone, cabazitaxel, and pembrolizumab. In other certain embodiments, the second agent is selected from the group consisting of enzalutamide and abiraterone. In yet other certain embodiments, the second agent is administered with a steroid. In one embodiment, the steroid comprises prednisone or methylprednisolone.

In some embodiments, the pharmaceutical compositions and the second agent are co-administered simultaneously. In other embodiments, the pharmaceutical composition(s) is/are administered prior to administration of the second agent. In other embodiments, the pharmaceutical composition(s) is/are administered after administration of the second agent. In yet other embodiments, the second agent is administered after the first pharmaceutical composition but before the second pharmaceutical composition. In some embodiments, the interval between administration of the pharmaceutical composition(s) and the second agent is about 1 hour, about 2 hours, about 3 hours, about 4 hours, about 5 hours, about 6 hours, about 7 hours, about 8 hours, about 9 hours, about 10 hours, about 11 hours, about 12 hours, about 1 day, about 2 days, about 3 days, about 4 days, about 5 days, about 6 days, about 1 week, about 8 days, about 9 days, about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, or more.

In some embodiments, the subject is suffering from, is susceptible to, or is at risk for prostate cancer.

In another aspect, provided herein is a kit comprising a container and an instruction for use, wherein the container comprises an arenavirus particle as identified above. In some embodiments, the arenavirus particle is in a pharmaceutical composition suitable for intravenous administration. In some embodiments, the kit further comprises an apparatus suitable for performing intravenous administration.

In another aspect, provided herein is a kit comprising two or more containers and an instruction for use, wherein one of the containers comprises an arenavirus particle as identified above, and another of the containers comprises a second agent. In some embodiments, the arenavirus particle is in a pharmaceutical composition suitable for intravenous administration. In some embodiments, the kit further comprises an apparatus suitable for performing intravenous administration.

### 3.4 Conventions and Abbreviations

| Abbreviation | Convention |
|---|---|
| GP | Glycoprotein |
| IGR | Intergenic region |
| JUNV | Junin virus |
| LCMV | Lymphocytic choriomeningitis virus |
| L protein | RNA-dependent RNA polymerase |
| L segment | Long segment |
| MHC | Major Histocompatibility Complex |
| Z protein | Matrix protein Z |
| NP | Nucleoprotein |
| ORF | Open reading frame |
| S segment | Short segment |
| UTR | Untranslated region |
| AE | Adverse event |
| CD8 | Cluster of differentiation 8 |
| CNS | Central nervous system |
| CT | Computed tomography |
| CTCAE | Common Terminology Criteria for |
| | Adverse Events |
| CTL | Cytotoxic T lymphocyte |
| DC | Dendritic cell |
| DCR | Disease control rate |
| DLT | Dose-limiting toxicity |
| DNA | Deoxyribonucleic acid |
| ECG | Electrocardiogram |
| ECOG | Eastern Cooperative Oncology Group |
| EOT | End of Treatment |
| FFU | Focus-forming units |
| HBsAg | Hepatitis B surface antigen |
| HCV | Hepatitis C virus |
| iCPD | Immune-confirmed progressive disease |
| ICS | Intracellular Cytokine Staining |
| IFN | Interferon |
| IL | Interleukin |
| iRECIST | Immune Response Evaluation Criteria |
| | in Solid Tumors |
| IV | Intravenous(ly) |
| MTD | Maximum tolerated dose |
| mCRPC | Metastatic castration resistant |
| | prostate cancer |
| MRI | Magnetic resonance imaging |
| N | Number of observations |
| ORR | Objective response rate |
| OS | Overall survival |
| PAP | Prostatic acid phosphatase |
| PCWG3 | Prostate cancer working group 3 |
| PET | Positron emission tomography |
| PFS | Progression-free survival |
| PICV | Pichinde virus |
| PMVS | Pre-master virus seed |
| PSA | Prostate specific antigen |
| PSMA | Prostate specific membrane antigen |
| RCV | Replication-competent virus |
| RECIST | Response Evaluation Criteria in |
| | Solid Tumors |
| RNA | Ribonucleic acid |
| RP2D | Recommended Phase II dose |
| SAE | Serious adverse event |
| TNF | Tumor necrosis factor |

### 4. BRIEF DESCRIPTION OF THE FIGURES

FIGS. 1A-IC: schematic illustration of the genetic composition of four exemplary tri-segmented arenavirus particles. (FIG. 1A) Schematic illustration of the genetic composition of artLCMV-PAP-NP/PSA-GP or artPICV-PAP-NP/PSA-GP; (FIG. 1B) Schematic illustration of the genetic composition of artLCMV-PSMA2-NP/PSMA1-GP; (FIG. 1C) Schematic illustration of the genetic composition of artPICV-PSMA1-NP/PSMA2-GP.

FIGS. 2A-2B: transgene stability of PMVS (12) of artLCMV-PAP-NP/PSA-GP. (FIG. 2A) PAP and PSA transgene stability was analyzed by PCR at indicated passage levels (P1, P5, or P10); (FIG. 2B) PAP and PSA transgene expression of PMVS (12) at indicated passages was confirmed by Western Blot analysis. Whole cell lysates of HEK/293VRC cells used for parental PMVS stock production and generation of passages were analyzed in Western Blots using PAP, PSA, LCMV NP and MAPK specific antibodies. Cell lysate of uninfected HEK/293VRC cells (control; C) or cell lysates from cells infected with R&D vector preparations of artLCMV-PAP-NP/PAP-GP (positive control 1; PC1) or R&D stock of artLCMV-PSA-NP/PSA-GP (positive control 2; PC2) were used as controls. Protein sizes (kDa) refer to peqGold protein standard V, PeqLab (M). PAP: 1162 bp=387 amino acids, PSA: 786 bp=262 amino acids.

FIGS. 3A-3B: transgene stability of PMVS(05) c132/05/05 of artPICV-PAP-NP/PSA-GP. (FIG. 3A) PAP and PSA transgene stability of PMVS(05) c132/05/05 was analyzed by PCR at indicated passage levels; (FIG. 3B) transgene expression of PMVS(05) c132/05/05 at indicated passage levels was confirmed by Western Blot analysis. PAP: 1162 bp=387 amino acids, PSA: 786 bp=262 amino acids. Cell lysate of uninfected HEK/293VRC cells (negative control, -c) or cell lysates from cells infected with R&D vector preparations of artPICV-PAP-NP/PSA-GP were used as controls (positive control, +c).

FIG. 4: transgene stability of artLCMV encoding full length PSMA on both S segments at indicated passages, tested by PCR. The expected size of the transgene encoded on the NP segment is 2532 bp, and the expected size of the transgene encoded on the GP segment is 2518 bp.

FIGS. 5A-5B: transgene stability of PMVS (09) Cl. 9/7/2 of artLCMV-PSMA2-NP/PSMA1-GP. (FIG. 5A) PSMA1 and PSMA2 transgene stability of PMVS (09) Cl. 9/7/2 was analyzed by PCR at indicated passage levels; (FIG. 5B) PSMA1 and PSMA2 transgene expression at indicated passages was analyzed by Western Blot analysis. The unavailability of strong and specific antibodies impeded signal detection for PSMA2 protein expression. Asterisk (*) highlights a band with weak signal found for the artPICV-PSMA1/2 positive control. Cell lysate of uninfected HEK/293VRC cells (negative control, -c) or cell lysates from cells infected with R&D vector preparations of artPICV-PSMA1-NP/PSMA2-GP (artPICV-PSMA1/2) or artPICV-PSMA-NP/PSMA-GP (artPICV-PSMA, encoding full length PSMA) were used as controls.

FIGS. 6A-6B: transgene stability of PMVS 26 of artPICV-PSMA1-NP/PSMA2-GP. (FIG. 6A) stability of transgenes encoded on NP and GP segments was analyzed by PCR at indicated passage levels; (FIG. 6B) Transgene expression at indicated passages was confirmed by Western Blot analysis.

FIGS. 7A-7E: induction of CD8 T cell response after administration of different arenavirus particles encoding prostate cancer-related antigens in mice. (FIG. 7A) CD8 T cell (i.e., IFN-γ+) responses against PSA and PAP in mice 7 days after single administration of indicated vectors. Peptide stimulation was performed with overlapping peptide libraries for PAP and PSA, respectively. Percentages of IFN-γ positive CD3+B220−CD8+ T cells are shown for individual mice, as arithmetic means±standard deviation; (FIG. 7B) CD8 T cell (i.e., IFN-γ+) responses against PSMA and subdomains of PSMA in mice 7 days after single administration of indicated vectors. Peptide stimulation was performed with an overlapping peptide library for PSMA or with the single peptides PSMA76-90 (PSMA1) or PSMA634-642 (PSMA2). Percentages of IFN-γ positive CD3+B220−CD8+ T cells are shown for individual mice, as arithmetic means±standard deviation; (FIG. 7C) CD8 T cell (i.e., IFN-γ+) responses against PAP and PSA in mice 7 days after single administration of indicated vector combinations. Peptide stimulation was performed with overlapping peptide libraries for PAP or PSA. Percentages of IFN-γ positive CD3+B220−CD8+ T cells are shown for individual mice, as arithmetic means±standard deviation; (FIG. 7D) CD8 T cell (i.e., IFN-γ+) responses against PSMA and subdomains of PSMA in mice 7 days after single administration of indicated vector combinations. Peptide stimulation was performed with an overlapping peptide library for PSMA or with the single peptides PSMA76-90 (PSMA1) or PSMA634-642 (PSMA2). Percentages of IFN-γ positive CD3+B220−CD8+ T cells are shown for individual mice, as arithmetic means±standard deviation; (FIG. 7E) CD8 T cell (i.e., IFN-γ+) responses against LCMV NP (left panel) and PICV NP (right panel) in mice 7 days after single administration of indicated vectors or vector combinations. Peptide stimulation was performed with overlapping peptide libraries for LCMV NP and PICV NP, respectively. Percentages of IFN-γ positive CD3+B220−CD8+ T cells are shown for individual mice, as arithmetic means±standard deviation.

FIGS. 8A-8C: immunogenicity of artLCMV-PAP-NP/PSA-GP and artPICV-PAP-NP/PSA-GP after homologous or heterologous alternating vector administration: (FIG. 8A) CD8 T cell (i.e., IFN-γ+) responses against PAP in mice, 26 days after the initial administration and 5 days after the sequential administration with indicated vectors. Peptide stimulation was performed with an overlapping peptide library for PAP. Percentages of IFN-γ positive CD3+B220−CD8+ T cells are shown for individual mice, as arithmetic means ±standard deviation; (FIG. 8B) CD8 T cell (i.e., IFN-γ+) responses against PSA in mice, 26 days after the initial administration and 5 days after the sequential administration of indicated vectors. Peptide stimulation was performed with an overlapping peptide library for PSA. Percentages of IFN-γ positive CD3+B220−CD8+ T cells are shown for individual mice, as arithmetic means±standard deviation; (FIG. 8C) CD8 T cell (i.e., IFN-γ+) responses against LCMV NP (left panel) and PICV NP (right panel) in mice 26 days after the initial administration and 5 days after the sequential administration with indicated vectors. Peptide stimulation was performed with overlapping peptide libraries for LCMV NP and PICV NP, respectively. Percentages of IFN-γ positive CD3+B220−CD8+ T cells are shown for individual mice, as arithmetic means±standard deviation.

FIGS. 9A-9B: immunogenicity of artLCMV-PSMA2-NP/PSMA1-GP and artPICV-PSMA1-NP/PSMA2-GP after homologous or heterologous alternating vector administration. (FIG. 9A) CD8 T cell (i.e., IFN-γ+) responses against PSMA and subdomains of PSMA in mice 26 days after the initial administration and 5 days after the sequential administration with indicated vectors. Peptide stimulation was performed with an overlapping peptide library for PSMA or with the single peptides PSMA₇₆₋₉₀ (PSMA1) or PSMA₆₃₄₋₆₄₂ (PSMA2). Percentages of IFN-γ positive CD3+B220−CD8+ T cells are shown for individual mice, as arithmetic means±standard deviation; (FIG. 9B) CD8 T cell (i.e., IFN-γ+) responses against LCMV NP (left panel) and PICV NP (right panel) in mice 26 days after the initial administration and 5 days after the sequential administration with indicated vectors. Peptide stimulation was performed with overlapping peptide libraries for LCMV NP and PICV NP, respectively. Percentages of IFN-γ positive CD3+B220−CD8+ T cells are shown for individual mice, as arithmetic means±standard deviation.

FIG. 10: immunogenicity of artLCMV and artPICV vector combinations after homologous or heterologous alternating vector administration. (FIG. 10A) CD8 T cell (i.e., IFN-γ+) responses against PAP in mice, 26 days after the initial administration and 5 days after the sequential administration with indicated vector mixes. Peptide stimulation was performed with an overlapping peptide library for PAP. Percentages of IFN-γ positive CD3+B220−CD8+ T cells are shown for individual mice, as arithmetic means±standard deviation; (FIG. 10B) CD8 T cell (i.e., IFN-γ+) responses against PSA in mice, 26 days after the initial administration and 5 days after the sequential administration with indicated vector mixes. Peptide stimulation was performed with an overlapping peptide library for PSA. Percentages of IFN-γ positive CD3+B220−CD8+ T cells are shown for individual mice, as arithmetic means±standard deviation; (FIG. 10C) CD8 T cell (i.e., IFN-γ+) responses against PSMA and subdomains of PSMA in mice 26 days after the initial administration and 5 days after the sequential administration with indicated vector mixes. Peptide stimulation was performed with an overlapping peptide library for PSMA or with the single peptides PSMA₇₆₋₉₀ (PSMA1) or PSMA₆₃₄₋₆₄₂ (PSMA2). Percentages of IFN-γ positive CD3+B220-CD8+ T cells are shown for individual mice, as arithmetic means±standard deviation; (FIG. 10D) CD8 T cell (i.e., IFN-γ+) responses against LCMV NP (left panel) and PICV NP (right panel) in mice 26 days after the initial administration and 5 days after the sequential administration with indicated vector mixes. Peptide stimulation was performed with overlapping peptide libraries for LCMV NP and PICV NP, respectively. Percentages of IFN-7 positive CD3+B220−CD8+ T cells are shown for individual mice, as arithmetic means standard deviation.

FIG. 11: Study Design for Dose Escalation and Dose Expansion. Abbreviations: Alt.=alternating, Approx=approximately, art=artificial, CRPC=castration resistant prostate cancer, GP=glycoprotein, IV=intravenous(ly), LCMV=Lymphocytic choriomeningitis virus, NP=nucleoprotein, PAP=prostatic acid phosphatase, PICV=Pichinde virus, PSA=Prostate specific antigen, PSMA=Prostate specific membrane antigen, RP2D=recommended Phase II dose, Seq.=sequential.

FIG. 12: Study Design Scheme. Abbreviations: DL1/2/3=dose level 1/2/3; mCRPC=Metastatic Castration Resistant Prostate Cancer; Ph2=Phase 2; RP2D=recommended Phase 2 dose.

### 5. DETAILED DESCRIPTION

Provided herein is a modified arenavirus genome segment which is engineered to carry a heterologous ORF encoding a prostate cancer-related antigen or an antigenic fragment thereof, as described in Section 5.1. Also provided herein is a modified tri-segmented arenavirus particle as described in Section 5.3 which contains arenavirus genome segments each carrying a heterologous ORF encoding a prostate cancer-related antigen or an antigenic fragment thereof, and a method of generating such an arenavirus particle, as described in Section 5.4. Also described herein are cDNA, DNA expression vectors, and host cells, as described in Section 5.2. Further provided herein is a pharmaceutical composition comprising such an arenavirus particle, as described in Section 5.5. Methods of treating prostate cancer using such an arenavirus particle are described in Section 5.6. Lastly, a variety of assays, such as the ones for detecting the modified arenavirus genome segment of an arenavirus particle described herein, and the ones for measuring immune response in treated animals, are described in Section 5.7.

### 5.1 Arenavirus Genome Segments

Provided herein are novel arenavirus genome segments having a heterologous ORF encoding a prostate cancer-related antigen. Such novel engineered arenavirus segments have a heterologous ORF encoding a prostate cancer-related antigen in addition to an arenavirus ORF encoding an arenavirus protein, such as the GP, NP, Z or L protein. Accordingly, in some embodiments, provided herein is an arenavirus S segment, wherein the arenavirus S segment is engineered to carry a heterologous ORF encoding PAP. In some embodiments, provided herein is an arenavirus S segment, wherein the arenavirus S segment is engineered to carry a heterologous ORF encoding PSA. In some embodiments, provided herein is an arenavirus S segment, wherein the arenavirus S segment is engineered to carry a heterologous ORF encoding PSMA. In some embodiments, provided herein is an arenavirus S segment, wherein the arenavirus S segment is engineered to carry a heterologous ORF encoding prostate stem cell antigen (PSCA). In some embodiments, provided herein is an arenavirus S segment, wherein the arenavirus S segment is engineered to carry a heterologous ORF encoding Mucin-1. In some embodiments, provided herein is an arenavirus S segment, wherein the arenavirus S segment is engineered to carry a heterologous ORF encoding NY-ESO-1. In some embodiments, provided herein is an arenavirus S segment, wherein the arenavirus S segment is engineered to carry a heterologous ORF encoding MAGE-A. In some embodiments, provided herein is an arenavirus S segment, wherein the arenavirus S segment is engineered to carry a heterologous ORF encoding AKAP-4.

As used herein, the term “antigenic fragment” is intended to refer to a portion of the antigen that either includes or corresponds to a sequential amino acid sequence or conformational immunologically active region that is sufficient to elicit an immune response against the antigen from which the antigenic fragment is derived. Such an immune response in the treated animals can be the same or similar to the immune response elicited by the original antigen from which the fragment is derived. An immune response elicited by an antigenic fragment includes detectable T-cell responses that are specific to the original antigen. Such antigenic fragments include amino acid sequences that are at least 500 amino acids, at least 490 amino acids, at least 480 amino acids, at least 470 amino acids, at least 460 amino acids, at least 450 amino acids, at least 440 amino acids, at least 430 amino acids, at least 420 amino acids, at least 410 amino acids, at least 400 amino acids, at least 390 amino acids, at least 380 amino acids, at least 370 amino acids, at least 360 amino acids, at least 350 amino acids, at least 340 amino acids, at least 330 amino acids, at least 320 amino acids, at least 310 amino acids, at least 300 amino acids, at least 290 amino acids, at least 280 amino acids, at least 270 amino acids, at least 260 amino acids, at least 250 amino acids, at least 240 amino acids, at least 230 amino acids, at least 220 amino acids, at least 210 amino acids, at least 200 amino acids, at least 190 amino acids, at least 180 amino acids, at least 170 amino acids, at least 160 amino acids, at least 150 amino acids, at least 100 amino acids, at least 50 amino acids in length, but less than the full length of the original antigen from which the fragment is derived.

Provided herein are novel arenavirus genome segments having a heterologous ORF encoding an antigenic fragment of a prostate cancer-related antigen. As such, in some embodiments, provided herein is an arenavirus S segment, wherein the arenavirus S segment is engineered to carry a heterologous ORF encoding an antigenic fragment of PAP. In some embodiments, provided herein is an arenavirus S segment, wherein the arenavirus S segment is engineered to carry a heterologous ORF encoding an antigenic fragment of PSA. In some embodiments, provided herein is an arenavirus S segment, wherein the arenavirus S segment is engineered to carry a heterologous ORF encoding an antigenic fragment of PSMA. In some embodiments, provided herein is an arenavirus S segment, wherein the arenavirus S segment is engineered to carry a heterologous ORF encoding an antigenic fragment of PSCA. In some embodiments, provided herein is an arenavirus S segment, wherein the arenavirus S segment is engineered to carry a heterologous ORF encoding an antigenic fragment of Mucin-1. In some embodiments, provided herein is an arenavirus S segment, wherein the arenavirus S segment is engineered to carry a heterologous ORF encoding an antigenic fragment of NY-ESO-1. In some embodiments, provided herein is an arenavirus S segment, wherein the arenavirus S segment is engineered to carry a heterologous ORF encoding an antigenic fragment of MAGE-A. In some embodiments, provided herein is an arenavirus S segment, wherein the arenavirus S segment is engineered to carry a heterologous ORF encoding an antigenic fragment of AKAP-4.

In some embodiments, provided herein are two arenavirus S segments each of which is engineered to carry a heterologous ORF encoding an antigenic fragment of PSMA, wherein the two antigenic fragments of PSMA comprise about half of the PSMA amino acid sequence. In some embodiments, such a fragment can be generated by taking advantage of a naturally existing ATG codon in the nucleotide sequence of the ORF. In other embodiments, such a fragment can be generated by artificially introducing ATG to the nucleotide sequence of PSMA.

Specifically, in some specific embodiments, the split of PSMA occurs right before a naturally existing ATG so that the second half of PSMA starts with the naturally existing ATG and is in frame with the translation of wild-type PSMA. Accordingly, in one embodiment, the split of PSMA generates the first half of PSMA that corresponds to 1^{st} to 171^{th} nucleotide of SEQ ID NO: 9 plus a stop codon added in the end, and the second half that corresponds to 172^{th} to 2253^{th} nucleotide of SEQ ID NO: 9. In another embodiment, the split of PSMA generates the first half of PSMA that corresponds to 1^{st} to 504^{th} nucleotide of SEQ ID NO: 9 plus a stop codon added in the end, and the second half that corresponds to 505^{th} to 2253^{th} nucleotide of SEQ ID NO: 9. In another embodiment, the split of PSMA generates the first half of PSMA that corresponds to 1^{st} to 573^{th} nucleotide of SEQ ID NO: 9 plus a stop codon added in the end, and the second half that corresponds to 5741^{th} to 2253^{th} nucleotide of SEQ ID NO: 9. In another embodiment, the split of PSMA generates the first half of PSMA that corresponds to 1^{st} to 924^{th} nucleotide of SEQ ID NO: 9 plus a stop codon added in the end, and the second half that corresponds to 925^{th} to 2253^{th} nucleotide of SEQ ID NO: 9. In another embodiment, the split of PSMA generates the first half of PSMA that corresponds to 1^{st} to 1029^{th} nucleotide of SEQ ID NO: 9 plus a stop codon added in the end, and the second half that corresponds to 1030^{th} to 2253^{th} nucleotide of SEQ ID NO: 9. In another embodiment, the split of PSMA generates the first half of PSMA that corresponds to 1^{st} to 1407^{th} nucleotide of SEQ ID NO: 9 plus a stop codon added in the end, and the second half that corresponds to 1408^{th} to 2253^{th} nucleotide of SEQ ID NO: 9. In another embodiment, the split of PSMA generates the first half of PSMA that corresponds to 1^{st} to 1524^{th} nucleotide of SEQ ID NO: 9 plus a stop codon added in the end, and the second half that corresponds to 1525^{th} to 2253^{th} nucleotide of SEQ ID NO: 9. In another embodiment, the split of PSMA generates the first half of PSMA that corresponds to 1^{st} to 1704^{th} nucleotide of SEQ ID NO: 9 plus a stop codon added in the end, and the second half that corresponds to 1705^{th} to 2253^{th} nucleotide of SEQ ID NO: 9. In another embodiment, the split of PSMA generates the first half of PSMA that corresponds to 1^{st} to 1746^{th} nucleotide of SEQ ID NO: 9 plus a stop codon added in the end, and the second half that corresponds to 1747^{th} to 2253^{th} nucleotide of SEQ ID NO: 9. In another embodiment, the split of PSMA generates the first half of PSMA that corresponds to 1^{st} to 1845′ nucleotide of SEQ ID NO: 9 plus a stop codon added in the end, and the second half that corresponds to 1846^{th} to 2253^{th} nucleotide of SEQ ID NO: 9. In another embodiment, the split of PSMA generates the first half of PSMA that corresponds to 1^{st} to 1863^{th} nucleotide of SEQ ID NO: 9 plus a stop codon added in the end, and the second half that corresponds to 1864^{th} to 2253^{th} nucleotide of SEQ ID NO: 9. In another embodiment, the split of PSMA generates the first half of PSMA that corresponds to 1^{st} to 1986^{th} nucleotide of SEQ ID NO: 9 plus a stop codon added in the end, and the second half that corresponds to 1987^{th} to 2253^{th} nucleotide of SEQ ID NO: 9. In another embodiment, the split of PSMA generates the first half of PSMA that corresponds to 1^{st} to 1989^{th} nucleotide of SEQ ID NO: 9 plus a stop codon added in the end, and the second half that corresponds to 1990^{th} to 2253^{th} nucleotide of SEQ ID NO: 9. In another embodiment, the split of PSMA generates the first half of PSMA that corresponds to 1st to 2004^{th} nucleotide of SEQ ID NO: 9 plus a stop codon added in the end, and the second half that corresponds to 2005^{th} to 2253^{th} nucleotide of SEQ ID NO: 9. In the above-described embodiments, the last codon of the first half of PSMA is mutated to be a stop codon. In the above-described embodiments, the last codon of the first half of PSMA can be TAG. In the above-described embodiments, the last codon of the first half of PSMA can be TAA. In the above-described embodiments, the last codon of the first half of PSMA can be TGA.

In some embodiments, the arenavirus S segment provided herein is engineered to carry a heterologous ORF encoding a prostate cancer-related antigen in a position under control of an arenavirus 5′ UTR, and an ORF encoding GP in a position under control of an arenavirus 3′ UTR. In other embodiments, the arenavirus S segment provided herein is engineered to carry a heterologous ORF encoding a prostate cancer-related antigen in a position under control of an arenavirus 3′ UTR, and an ORF encoding GP in a position under control of an arenavirus 5′ UTR. In other embodiments, the arenavirus S segment provided herein is engineered to carry a heterologous ORF encoding a prostate cancer-related antigen in a position under control of an arenavirus 5′ UTR, and an ORF encoding NP in a position under control of an arenavirus 3′ UTR. In other embodiments, the arenavirus S segment provided herein is engineered to carry a heterologous ORF encoding a prostate cancer-related antigen in a position under control of an arenavirus 3′ UTR, and an ORF encoding NP in a position under control of an arenavirus 5′ UTR. In other embodiments, the arenavirus S segment provided herein is engineered to carry a heterologous ORF encoding a prostate cancer-related antigen in a position under control of an arenavirus 5′ UTR, and an ORF encoding Z in a position under control of an arenavirus 3′ UTR. In other embodiments, the arenavirus S segment provided herein is engineered to carry a heterologous ORF encoding a prostate cancer-related antigen in a position under control of an arenavirus 3′ UTR, and an ORF encoding Z in a position under control of an arenavirus 5′ UTR. In other embodiments, the arenavirus S segment provided herein is engineered to carry a heterologous ORF encoding a prostate cancer-related antigen in a position under control of an arenavirus 5′ UTR, and an ORF encoding L in a position under control of an arenavirus 3′ UTR. In other embodiments, the arenavirus S segment provided herein is engineered to carry a heterologous ORF encoding a prostate cancer-related antigen in a position under control of an arenavirus 3′ UTR, and an ORF encoding L in a position under control of an arenavirus 5′ UTR.

In some embodiments, the arenavirus S segment provided herein is engineered to carry a heterologous ORF encoding an antigenic fragment of a prostate cancer-related antigen in a position under control of an arenavirus 5′ UTR, and an ORF encoding GP in a position under control of an arenavirus 3′ UTR. In other embodiments, the arenavirus S segment provided herein is engineered to carry a heterologous ORF encoding an antigenic fragment of a prostate cancer-related antigen in a position under control of an arenavirus 3′ UTR, and an ORF encoding GP in a position under control of an arenavirus 5′ UTR. In other embodiments, the arenavirus S segment provided herein is engineered to carry a heterologous ORF encoding an antigenic fragment of a prostate cancer-related antigen in a position under control of an arenavirus 5′ UTR, and an ORF encoding NP in a position under control of an arenavirus 3′ UTR. In other embodiments, the arenavirus S segment provided herein is engineered to carry a heterologous ORF encoding an antigenic fragment of a prostate cancer-related antigen in a position under control of an arenavirus 3′ UTR, and an ORF encoding NP in a position under control of an arenavirus 5′ UTR. In other embodiments, the arenavirus S segment provided herein is engineered to carry a heterologous ORF encoding an antigenic fragment of a prostate cancer-related antigen in a position under control of an arenavirus 5′ UTR, and an ORF encoding Z in a position under control of an arenavirus 3′ UTR. In other embodiments, the arenavirus S segment provided herein is engineered to carry a heterologous ORF encoding an antigenic fragment of a prostate cancer-related antigen in a position under control of an arenavirus 3′ UTR, and an ORF encoding Z in a position under control of an arenavirus 5′ UTR. In other embodiments, the arenavirus S segment provided herein is engineered to carry a heterologous ORF encoding an antigenic fragment of a prostate cancer-related antigen in a position under control of an arenavirus 5′ UTR, and an ORF encoding L in a position under control of an arenavirus 3′ UTR. In other embodiments, the arenavirus S segment provided herein is engineered to carry a heterologous ORF encoding an antigenic fragment of a prostate cancer-related antigen in a position under control of an arenavirus 3′ UTR, and an ORF encoding L in a position under control of an arenavirus 5′ UTR.

In some embodiments, the arenavirus S segment provided herein is engineered to carry a heterologous ORF encoding PAP as described herein in a position under control of an arenavirus 5′ UTR, and an ORF encoding GP in a position under control of an arenavirus 3′ UTR. In other embodiments, the arenavirus S segment provided herein is engineered to carry a heterologous ORF encoding PAP as described herein in a position under control of an arenavirus 3′ UTR, and an ORF encoding GP in a position under control of an arenavirus 5′ UTR. In other embodiments, the arenavirus S segment provided herein is engineered to carry a heterologous ORF encoding PAP as described herein in a position under control of an arenavirus 5′ UTR, and an ORF encoding NP in a position under control of an arenavirus 3′ UTR. In other embodiments, the arenavirus S segment provided herein is engineered to carry a heterologous ORF encoding PAP as described herein in a position under control of an arenavirus 3′ UTR, and an ORF encoding NP in a position under control of an arenavirus 5′ UTR. In other embodiments, the arenavirus S segment provided herein is engineered to carry a heterologous ORF encoding PAP as described herein in a position under control of an arenavirus 5′ UTR, and an ORF encoding Z in a position under control of an arenavirus 3′ UTR. In other embodiments, the arenavirus S segment provided herein is engineered to carry a heterologous ORF encoding PAP as described herein in a position under control of an arenavirus 3′ UTR, and an ORF encoding Z in a position under control of an arenavirus 5′ UTR. In other embodiments, the arenavirus S segment provided herein is engineered to carry a heterologous ORF encoding PAP as described herein in a position under control of an arenavirus 5′ UTR, and an ORF encoding L in a position under control of an arenavirus 3′ UTR. In other embodiments, the arenavirus S segment provided herein is engineered to carry a heterologous ORF encoding PAP as described herein in a position under control of an arenavirus 3′ UTR, and an ORF encoding L in a position under control of an arenavirus 5′ UTR.

In some embodiments, the arenavirus S segment provided herein is engineered to carry a heterologous ORF encoding an antigenic fragment of PAP as described herein in a position under control of an arenavirus 5′ UTR, and an ORF encoding GP in a position under control of an arenavirus 3′ UTR. In other embodiments, the arenavirus S segment provided herein is engineered to carry a heterologous ORF encoding an antigenic fragment of PAP as described herein in a position under control of an arenavirus 3′ UTR, and an ORF encoding GP in a position under control of an arenavirus 5′ UTR. In other embodiments, the arenavirus S segment provided herein is engineered to carry a heterologous ORF encoding an antigenic fragment of PAP as described herein in a position under control of an arenavirus 5′ UTR, and an ORF encoding NP in a position under control of an arenavirus 3′ UTR. In other embodiments, the arenavirus S segment provided herein is engineered to carry a heterologous ORF encoding an antigenic fragment of PAP as described herein in a position under control of an arenavirus 3′ UTR, and an ORF encoding NP in a position under control of an arenavirus 5′ UTR. In other embodiments, the arenavirus S segment provided herein is engineered to carry a heterologous ORF encoding an antigenic fragment of PAP as described herein in a position under control of an arenavirus 5′ UTR, and an ORF encoding Z in a position under control of an arenavirus 3′ UTR. In other embodiments, the arenavirus S segment provided herein is engineered to carry a heterologous ORF encoding an antigenic fragment of PAP as described herein in a position under control of an arenavirus 3′ UTR, and an ORF encoding Z in a position under control of an arenavirus 5′ UTR. In other embodiments, the arenavirus S segment provided herein is engineered to carry a heterologous ORF encoding an antigenic fragment of PAP as described herein in a position under control of an arenavirus 5′ UTR, and an ORF encoding L in a position under control of an arenavirus 3′ UTR. In other embodiments, the arenavirus S segment provided herein is engineered to carry a heterologous ORF encoding an antigenic fragment of PAP as described herein in a position under control of an arenavirus 3′ UTR, and an ORF encoding L in a position under control of an arenavirus 5′ UTR.

In some embodiments, the arenavirus S segment provided herein is engineered to carry a heterologous ORF encoding PSA as described herein in a position under control of an arenavirus 5′ UTR, and an ORF encoding GP in a position under control of an arenavirus 3′ UTR. In other embodiments, the arenavirus S segment provided herein is engineered to carry a heterologous ORF encoding PSA as described herein in a position under control of an arenavirus 3′ UTR, and an ORF encoding GP in a position under control of an arenavirus 5′ UTR. In other embodiments, the arenavirus S segment provided herein is engineered to carry a heterologous ORF encoding PSA as described herein in a position under control of an arenavirus 5′ UTR, and an ORF encoding NP in a position under control of an arenavirus 3′ UTR. In other embodiments, the arenavirus S segment provided herein is engineered to carry a heterologous ORF encoding PSA as described herein in a position under control of an arenavirus 3′ UTR, and an ORF encoding NP in a position under control of an arenavirus 5′ UTR. In other embodiments, the arenavirus S segment provided herein is engineered to carry a heterologous ORF encoding PSA as described herein in a position under control of an arenavirus 5′ UTR, and an ORF encoding Z in a position under control of an arenavirus 3′ UTR. In other embodiments, the arenavirus S segment provided herein is engineered to carry a heterologous ORF encoding PSA as described herein in a position under control of an arenavirus 3′ UTR, and an ORF encoding Z in a position under control of an arenavirus 5′ UTR. In other embodiments, the arenavirus S segment provided herein is engineered to carry a heterologous ORF encoding PSA as described herein in a position under control of an arenavirus 5′ UTR, and an ORF encoding L in a position under control of an arenavirus 3′ UTR. In other embodiments, the arenavirus S segment provided herein is engineered to carry a heterologous ORF encoding PSA as described herein in a position under control of an arenavirus 3′ UTR, and an ORF encoding L in a position under control of an arenavirus 5′ UTR.

In some embodiments, the arenavirus S segment provided herein is engineered to carry a heterologous ORF encoding an antigenic fragment of PSA as described herein in a position under control of an arenavirus 5′ UTR, and an ORF encoding GP in a position under control of an arenavirus 3′ UTR. In other embodiments, the arenavirus S segment provided herein is engineered to carry a heterologous ORF encoding an antigenic fragment of PSA as described herein in a position under control of an arenavirus 3′ UTR, and an ORF encoding GP in a position under control of an arenavirus 5′ UTR. In other embodiments, the arenavirus S segment provided herein is engineered to carry a heterologous ORF encoding an antigenic fragment of PSA as described herein in a position under control of an arenavirus 5′ UTR, and an ORF encoding NP in a position under control of an arenavirus 3′ UTR. In other embodiments, the arenavirus S segment provided herein is engineered to carry a heterologous ORF encoding an antigenic fragment of PSA as described herein in a position under control of an arenavirus 3′ UTR, and an ORF encoding NP in a position under control of an arenavirus 5′ UTR. In other embodiments, the arenavirus S segment provided herein is engineered to carry a heterologous ORF encoding an antigenic fragment of PSA as described herein in a position under control of an arenavirus 5′ UTR, and an ORF encoding Z in a position under control of an arenavirus 3′ UTR. In other embodiments, the arenavirus S segment provided herein is engineered to carry a heterologous ORF encoding an antigenic fragment of PSA as described herein in a position under control of an arenavirus 3′ UTR, and an ORF encoding Z in a position under control of an arenavirus 5′ UTR. In other embodiments, the arenavirus S segment provided herein is engineered to carry a heterologous ORF encoding an antigenic fragment of PSA as described herein in a position under control of an arenavirus 5′ UTR, and an ORF encoding L in a position under control of an arenavirus 3′ UTR. In other embodiments, the arenavirus S segment provided herein is engineered to carry a heterologous ORF encoding an antigenic fragment of PSA as described herein in a position under control of an arenavirus 3′ UTR, and an ORF encoding L in a position under control of an arenavirus 5′ UTR.

In some embodiments, the arenavirus S segment provided herein is engineered to carry a heterologous ORF encoding PSMA as described herein in a position under control of an arenavirus 5′ UTR, and an ORF encoding GP in a position under control of an arenavirus 3′ UTR. In other embodiments, the arenavirus S segment provided herein is engineered to carry a heterologous ORF encoding PSMA as described herein in a position under control of an arenavirus 3′ UTR, and an ORF encoding GP in a position under control of an arenavirus 5′ UTR. In other embodiments, the arenavirus S segment provided herein is engineered to carry a heterologous ORF encoding PSMA as described herein in a position under control of an arenavirus 5′ UTR, and an ORF encoding NP in a position under control of an arenavirus 3′ UTR. In other embodiments, the arenavirus S segment provided herein is engineered to carry a heterologous ORF encoding PSMA as described herein in a position under control of an arenavirus 3′ UTR, and an ORF encoding NP in a position under control of an arenavirus 5′ UTR. In other embodiments, the arenavirus S segment provided herein is engineered to carry a heterologous ORF encoding PSMA as described herein in a position under control of an arenavirus 5′ UTR, and an ORF encoding Z in a position under control of an arenavirus 3′ UTR. In other embodiments, the arenavirus S segment provided herein is engineered to carry a heterologous ORF encoding PSMA as described herein in a position under control of an arenavirus 3′ UTR, and an ORF encoding Z in a position under control of an arenavirus 5′ UTR. In other embodiments, the arenavirus S segment provided herein is engineered to carry a heterologous ORF encoding PSMA as described herein in a position under control of an arenavirus 5′ UTR, and an ORF encoding L in a position under control of an arenavirus 3′ UTR. In other embodiments, the arenavirus S segment provided herein is engineered to carry a heterologous ORF encoding PSMA as described herein in a position under control of an arenavirus 3′ UTR, and an ORF encoding L in a position under control of an arenavirus 5′ UTR. In some embodiments, the arenavirus S segment provided herein is engineered to carry a heterologous ORF encoding an antigenic fragment of PSMA as described herein in a position under control of an arenavirus 5′ UTR, and an ORF encoding GP in a position under control of an arenavirus 3′ UTR. In other embodiments, the arenavirus S segment provided herein is engineered to carry a heterologous ORF encoding an antigenic fragment of PSMA as described herein in a position under control of an arenavirus 3′ UTR, and an ORF encoding GP in a position under control of an arenavirus 5′ UTR. In other embodiments, the arenavirus S segment provided herein is engineered to carry a heterologous ORF encoding an antigenic fragment of PSMA as described herein in a position under control of an arenavirus 5′ UTR, and an ORF encoding NP in a position under control of an arenavirus 3′ UTR. In other embodiments, the arenavirus S segment provided herein is engineered to carry a heterologous ORF encoding an antigenic fragment of PSMA as described herein in a position under control of an arenavirus 3′ UTR, and an ORF encoding NP in a position under control of an arenavirus 5′ UTR. In other embodiments, the arenavirus S segment provided herein is engineered to carry a heterologous ORF encoding an antigenic fragment of PSMA as described herein in a position under control of an arenavirus 5′ UTR, and an ORF encoding Z in a position under control of an arenavirus 3′ UTR. In other embodiments, the arenavirus S segment provided herein is engineered to carry a heterologous ORF encoding an antigenic fragment of PSMA as described herein in a position under control of an arenavirus 3′ UTR, and an ORF encoding Z in a position under control of an arenavirus 5′ UTR. In other embodiments, the arenavirus S segment provided herein is engineered to carry a heterologous ORF encoding an antigenic fragment of PSMA as described herein in a position under control of an arenavirus 5′ UTR, and an ORF encoding L in a position under control of an arenavirus 3′ UTR. In other embodiments, the arenavirus S segment provided herein is engineered to carry a heterologous ORF encoding an antigenic fragment of PSMA as described herein in a position under control of an arenavirus 3′ UTR, and an ORF encoding L in a position under control of an arenavirus 5′ UTR.

In some embodiments, the arenavirus S segment provided herein is engineered to carry a heterologous ORF encoding PSCA, Mucin-1, NY-ESO-1, MAGE-A, or AKAP-4 as described herein in a position under control of an arenavirus 5′ UTR, and an ORF encoding GP in a position under control of an arenavirus 3′ UTR. In other embodiments, the arenavirus S segment provided herein is engineered to carry a heterologous ORF encoding PSCA, Mucin-1, NY-ESO-1, MAGE-A, or AKAP-4 as described herein in a position under control of an arenavirus 3′ UTR, and an ORF encoding GP in a position under control of an arenavirus 5′ UTR. In other embodiments, the arenavirus S segment provided herein is engineered to carry a heterologous ORF encoding PSCA, Mucin-1, NY-ESO-1, MAGE-A, or AKAP-4 as described herein in a position under control of an arenavirus 5′ UTR, and an ORF encoding NP in a position under control of an arenavirus 3′ UTR. In other embodiments, the arenavirus S segment provided herein is engineered to carry a heterologous ORF encoding PSCA, Mucin-1, NY-ESO-1, MAGE-A, or AKAP-4 as described herein in a position under control of an arenavirus 3′ UTR, and an ORF encoding NP in a position under control of an arenavirus 5′ UTR. In other embodiments, the arenavirus S segment provided herein is engineered to carry a heterologous ORF encoding PSCA, Mucin-1, NY-ESO-1, MAGE-A, or AKAP-4 as described herein in a position under control of an arenavirus 5′ UTR, and an ORF encoding Z in a position under control of an arenavirus 3′ UTR. In other embodiments, the arenavirus S segment provided herein is engineered to carry a heterologous ORF encoding PSCA, Mucin-1, NY-ESO-1, MAGE-A, or AKAP-4 as described herein in a position under control of an arenavirus 3′ UTR, and an ORF encoding Z in a position under control of an arenavirus 5′ UTR. In other embodiments, the arenavirus S segment provided herein is engineered to carry a heterologous ORF encoding PSCA, Mucin-1, NY-ESO-1, MAGE-A, or AKAP-4 as described herein in a position under control of an arenavirus 5′ UTR, and an ORF encoding L in a position under control of an arenavirus 3′ UTR. In other embodiments, the arenavirus S segment provided herein is engineered to carry a heterologous ORF encoding PSCA, Mucin-1, NY-ESO-1, MAGE-A, or AKAP-4 as described herein in a position under control of an arenavirus 3′ UTR, and an ORF encoding L in a position under control of an arenavirus 5′ UTR.

In some embodiments, the arenavirus S segment provided herein is engineered to carry a heterologous ORF encoding an antigenic fragment of PSCA, Mucin-1, NY-ESO-1, MAGE-A, or AKAP-4 as described herein in a position under control of an arenavirus 5′ UTR, and an ORF encoding GP in a position under control of an arenavirus 3′ UTR. In other embodiments, the arenavirus S segment provided herein is engineered to carry a heterologous ORF encoding an antigenic fragment of PSCA, Mucin-1, NY-ESO-1, MAGE-A, or AKAP-4 as described herein in a position under control of an arenavirus 3′ UTR, and an ORF encoding GP in a position under control of an arenavirus 5′ UTR. In other embodiments, the arenavirus S segment provided herein is engineered to carry a heterologous ORF encoding an antigenic fragment of PSCA, Mucin-1, NY-ESO-1, MAGE-A, or AKAP-4 as described herein in a position under control of an arenavirus 5′ UTR, and an ORF encoding NP in a position under control of an arenavirus 3′ UTR. In other embodiments, the arenavirus S segment provided herein is engineered to carry a heterologous ORF encoding an antigenic fragment of PSCA, Mucin-1, NY-ESO-1, MAGE-A, or AKAP-4 as described herein in a position under control of an arenavirus 3′ UTR, and an ORF encoding NP in a position under control of an arenavirus 5′ UTR. In other embodiments, the arenavirus S segment provided herein is engineered to carry a heterologous ORF encoding an antigenic fragment of PSCA, Mucin-1, NY-ESO-1, MAGE-A, or AKAP-4 as described herein in a position under control of an arenavirus 5′ UTR, and an ORF encoding Z in a position under control of an arenavirus 3′ UTR. In other embodiments, the arenavirus S segment provided herein is engineered to carry a heterologous ORF encoding an antigenic fragment of PSCA, Mucin-1, NY-ESO-1, MAGE-A, or AKAP-4 as described herein in a position under control of an arenavirus 3′ UTR, and an ORF encoding Z in a position under control of an arenavirus 5′ UTR. In other embodiments, the arenavirus S segment provided herein is engineered to carry a heterologous ORF encoding an antigenic fragment of PSCA, Mucin-1, NY-ESO-1, MAGE-A, or AKAP-4 as described herein in a position under control of an arenavirus 5′ UTR, and an ORF encoding L in a position under control of an arenavirus 3′ UTR. In other embodiments, the arenavirus S segment provided herein is engineered to carry a heterologous ORF encoding an antigenic fragment of PSCA, Mucin-1, NY-ESO-1, MAGE-A, or AKAP-4 as described herein in a position under control of an arenavirus 3′ UTR, and an ORF encoding L in a position under control of an arenavirus 5′ UTR.

Provided herein are amino acid sequences of a prostate cancer-related antigen or an antigenic fragment thereof. Accordingly, in some embodiments, the amino acid sequence of PAP encoded by the heterologous ORF described herein has at least 50% sequence identity to SEQ ID NO: 1. In some embodiments, the amino acid sequence of PAP encoded by the heterologous ORF described herein has at least 55% sequence identity to SEQ ID NO: 1. In some embodiments, the amino acid sequence of PAP encoded by the heterologous ORF described herein has at least 60% sequence identity to SEQ ID NO: 1. In some embodiments, the amino acid sequence of PAP encoded by the heterologous ORF described herein has at least 65% sequence identity to SEQ ID NO: 1. In some embodiments, the amino acid sequence of PAP encoded by the heterologous ORF described herein has at least 70% sequence identity to SEQ ID NO: 1. In some embodiments, the amino acid sequence of PAP encoded by the heterologous ORF described herein has at least 75% sequence identity to SEQ ID NO: 1. In some embodiments, the amino acid sequence of PAP encoded by the heterologous ORF described herein has at least 80% sequence identity to SEQ ID NO: 1. In some embodiments, the amino acid sequence of PAP encoded by the heterologous ORF described herein has at least 85% sequence identity to SEQ ID NO: 1. In some embodiments, the amino acid sequence of PAP encoded by the heterologous ORF described herein has at least 90% sequence identity to SEQ ID NO: 1. In some embodiments, the amino acid sequence of PAP encoded by the heterologous ORF described herein has at least 91% sequence identity to SEQ ID NO: 1. In some embodiments, the amino acid sequence of PAP encoded by the heterologous ORF described herein has at least 92% sequence identity to SEQ ID NO: 1. In some embodiments, the amino acid sequence of PAP encoded by the heterologous ORF described herein has at least 93% sequence identity to SEQ ID NO: 1. In some embodiments, the amino acid sequence of PAP encoded by the heterologous ORF described herein has at least 94% sequence identity to SEQ ID NO: 1. In some embodiments, the amino acid sequence of PAP encoded by the heterologous ORF described herein has at least 95% sequence identity to SEQ ID NO: 1. In some embodiments, the amino acid sequence of PAP encoded by the heterologous ORF described herein has at least 96% sequence identity to SEQ ID NO: 1. In some embodiments, the amino acid sequence of PAP encoded by the heterologous ORF described herein has at least 97% sequence identity to SEQ ID NO: 1. In some embodiments, the amino acid sequence of PAP encoded by the heterologous ORF described herein has at least 98% sequence identity to SEQ ID NO: 1. In some embodiments, the amino acid sequence of PAP encoded by the heterologous ORF described herein has at least 99% sequence identity to SEQ ID NO: 1. In some embodiments, the amino acid sequence of PAP encoded by the heterologous ORF described herein consists of SEQ ID NO: 1. In some embodiments, the amino acid sequence of PAP encoded by the heterologous ORF described herein comprises SEQ ID NO: 1. In some embodiments, the amino acid sequence of PAP described herein possesses one or more amino acid substitutions of SEQ ID NO: 1. In some embodiments, the amino acid sequence of PAP described herein possesses an amino acid substitution of SEQ ID NO: 1. In some embodiments, the amino acid substitution is substitution of Isoleucine for Arginine at the amino acid position 2 of SEQ ID NO: 1 (i.e., an I2R mutation). In some embodiments, the amino acid sequence of PAP described herein comprises SEQ ID NO: 18. In some embodiments, the amino acid sequence of PAP described herein consists of SEQ ID NO: 18.

In some embodiments, the amino acid sequence of PSA encoded by the heterologous ORF described herein has at least 50% sequence identity to SEQ ID NO: 2. In some embodiments, the amino acid sequence of PSA encoded by the heterologous ORF described herein has at least 55% sequence identity to SEQ ID NO: 2. In some embodiments, the amino acid sequence of PSA encoded by the heterologous ORF described herein has at least 60% sequence identity to SEQ ID NO: 2. In some embodiments, the amino acid sequence of PSA encoded by the heterologous ORF described herein has at least 65% sequence identity to SEQ ID NO: 2. In some embodiments, the amino acid sequence of PSA encoded by the heterologous ORF described herein has at least 70% sequence identity to SEQ ID NO: 2. In some embodiments, the amino acid sequence of PSA encoded by the heterologous ORF described herein has at least 75% sequence identity to SEQ ID NO: 2. In some embodiments, the amino acid sequence of PSA encoded by the heterologous ORF described herein has at least 80% sequence identity to SEQ ID NO: 2. In some embodiments, the amino acid sequence of PSA encoded by the heterologous ORF described herein has at least 85% sequence identity to SEQ ID NO: 2. In some embodiments, the amino acid sequence of PSA encoded by the heterologous ORF described herein has at least 90% sequence identity to SEQ ID NO: 2. In some embodiments, the amino acid sequence of PSA encoded by the heterologous ORF described herein has at least 91% sequence identity to SEQ ID NO: 2. In some embodiments, the amino acid sequence of PSA encoded by the heterologous ORF described herein has at least 92% sequence identity to SEQ ID NO: 2. In some embodiments, the amino acid sequence of PSA encoded by the heterologous ORF described herein has at least 93% sequence identity to SEQ ID NO: 2. In some embodiments, the amino acid sequence of PSA encoded by the heterologous ORF described herein has at least 94% sequence identity to SEQ ID NO: 2. In some embodiments, the amino acid sequence of PSA encoded by the heterologous ORF described herein has at least 95% sequence identity to SEQ ID NO: 2. In some embodiments, the amino acid sequence of PSA encoded by the heterologous ORF described herein has at least 96% sequence identity to SEQ ID NO: 2. In some embodiments, the amino acid sequence of PSA encoded by the heterologous ORF described herein has at least 97% sequence identity to SEQ ID NO: 2. In some embodiments, the amino acid sequence of PSA encoded by the heterologous ORF described herein has at least 98% sequence identity to SEQ ID NO: 2. In some embodiments, the amino acid sequence of PSA encoded by the heterologous ORF described herein has at least 99% sequence identity to SEQ ID NO: 2. In some embodiments, the amino acid sequence of PSA encoded by the heterologous ORF described herein consists of SEQ ID NO: 2. In some embodiments, the amino acid sequence of PSA encoded by the heterologous ORF described herein comprises SEQ ID NO: 2.

In some embodiments, the amino acid sequence of the antigenic fragment of PSMA encoded by the heterologous ORF described herein has at least 50% sequence identity to SEQ ID NO: 3 or 4. In some embodiments, the amino acid sequence of the antigenic fragment of PSMA encoded by the heterologous ORF described herein has at least 55% sequence identity to SEQ ID NO: 3 or 4. In some embodiments, the amino acid sequence of the antigenic fragment of PSMA encoded by the heterologous ORF described herein has at least 60% sequence identity to SEQ ID NO: 3 or 4. In some embodiments, the amino acid sequence of the antigenic fragment of PSMA encoded by the heterologous ORF described herein has at least 65% sequence identity to SEQ ID NO: 3 or 4. In some embodiments, the amino acid sequence of the antigenic fragment of PSMA encoded by the heterologous ORF described herein has at least 70% sequence identity to SEQ ID NO: 3 or 4. In some embodiments, the amino acid sequence of the antigenic fragment of PSMA encoded by the heterologous ORF described herein has at least 75% sequence identity to SEQ ID NO: 3 or 4. In some embodiments, the amino acid sequence of the antigenic fragment of PSMA encoded by the heterologous ORF described herein has at least 80% sequence identity to SEQ ID NO: 3 or 4. In some embodiments, the amino acid sequence of the antigenic fragment of PSMA encoded by the heterologous ORF described herein has at least 85% sequence identity to SEQ ID NO: 3 or 4. In some embodiments, the amino acid sequence of the antigenic fragment of PSMA encoded by the heterologous ORF described herein has at least 90% sequence identity to SEQ ID NO: 3 or 4. In some embodiments, the amino acid sequence of the antigenic fragment of PSMA encoded by the heterologous ORF described herein has at least 91% sequence identity to SEQ ID NO: 3 or 4. In some embodiments, the amino acid sequence of the antigenic fragment of PSMA encoded by the heterologous ORF described herein has at least 92% sequence identity to SEQ ID NO: 3 or 4. In some embodiments, the amino acid sequence of the antigenic fragment of PSMA encoded by the heterologous ORF described herein has at least 93% sequence identity to SEQ ID NO: 3 or 4. In some embodiments, the amino acid sequence of the antigenic fragment of PSMA encoded by the heterologous ORF described herein has at least 94% sequence identity to SEQ ID NO: 3 or 4. In some embodiments, the amino acid sequence of the antigenic fragment of PSMA encoded by the heterologous ORF described herein has at least 95% sequence identity to SEQ ID NO: 3 or 4. In some embodiments, the amino acid sequence of the antigenic fragment of PSMA encoded by the heterologous ORF described herein has at least 96% sequence identity to SEQ ID NO: 3 or 4. In some embodiments, the amino acid sequence of the antigenic fragment of PSMA encoded by the heterologous ORF described herein has at least 97% sequence identity to SEQ ID NO: 3 or 4. In some embodiments, the amino acid sequence of the antigenic fragment of PSMA encoded by the heterologous ORF described herein has at least 98% sequence identity to SEQ ID NO: 3 or 4. In some embodiments, the amino acid sequence of the antigenic fragment of PSMA encoded by the heterologous ORF described herein has at least 99% sequence identity to SEQ ID NO: 3 or 4. In some embodiments, the amino acid sequence of the antigenic fragment of PSMA encoded by the heterologous ORF described herein consists of SEQ ID NO: 3 or 4. In some embodiments, the amino acid sequence of the antigenic fragment of PSMA encoded by the heterologous ORF described herein comprises SEQ ID NO: 3 or 4.

Provided herein are nucleotide sequences encoding a prostate cancer-related antigen or an antigenic fragment thereof. Accordingly, in some embodiments, the nucleotide sequence of PAP encoded by the heterologous ORF described herein has at least 50% sequence identity to SEQ ID NO: 5. In some embodiments, the nucleotide sequence of PAP encoded by the heterologous ORF described herein has at least 55% sequence identity to SEQ ID NO: 5. In some embodiments, the nucleotide sequence of PAP encoded by the heterologous ORF described herein has at least 60% sequence identity to SEQ ID NO: 5. In some embodiments, the nucleotide sequence of PAP encoded by the heterologous ORF described herein has at least 65% sequence identity to SEQ ID NO: 5. In some embodiments, the nucleotide sequence of PAP encoded by the heterologous ORF described herein has at least 70% sequence identity to SEQ ID NO: 5. In some embodiments, the nucleotide sequence of PAP encoded by the heterologous ORF described herein has at least 75% sequence identity to SEQ ID NO: 5. In some embodiments, the nucleotide sequence of PAP encoded by the heterologous ORF described herein has at least 80% sequence identity to SEQ ID NO: 5. In some embodiments, the nucleotide sequence of PAP encoded by the heterologous ORF described herein has at least 85% sequence identity to SEQ ID NO: 5. In some embodiments, the nucleotide sequence of PAP encoded by the heterologous ORF described herein has at least 90% sequence identity to SEQ ID NO: 5. In some embodiments, the nucleotide sequence of PAP encoded by the heterologous ORF described herein has at least 91% sequence identity to SEQ ID NO: 5. In some embodiments, the nucleotide sequence of PAP encoded by the heterologous ORF described herein has at least 92% sequence identity to SEQ ID NO: 5. In some embodiments, the nucleotide sequence of PAP encoded by the heterologous ORF described herein has at least 93% sequence identity to SEQ ID NO: 5. In some embodiments, the nucleotide sequence of PAP encoded by the heterologous ORF described herein has at least 94% sequence identity to SEQ ID NO: 5. In some embodiments, the nucleotide sequence of PAP encoded by the heterologous ORF described herein has at least 95% sequence identity to SEQ ID NO: 5. In some embodiments, the nucleotide sequence of PAP encoded by the heterologous ORF described herein has at least 96% sequence identity to SEQ ID NO: 5. In some embodiments, the nucleotide sequence of PAP encoded by the heterologous ORF described herein has at least 97% sequence identity to SEQ ID NO: 5. In some embodiments, the nucleotide sequence of PAP encoded by the heterologous ORF described herein has at least 98% sequence identity to SEQ ID NO: 5. In some embodiments, the nucleotide sequence of PAP encoded by the heterologous ORF described herein has at least 99% sequence identity to SEQ ID NO: 5. In some embodiments, the nucleotide sequence of PAP encoded by the heterologous ORF described herein consists of SEQ ID NO: 5. In some embodiments, the nucleotide sequence of PAP encoded by the heterologous ORF described herein comprises SEQ ID NO: 5.

In some embodiments, the nucleotide sequence of PSA encoded by the heterologous ORF described herein has at least 50% sequence identity to SEQ ID NO: 6. In some embodiments, the nucleotide sequence of PSA encoded by the heterologous ORF described herein has at least 55% sequence identity to SEQ ID NO: 6. In some embodiments, the nucleotide sequence of PSA encoded by the heterologous ORF described herein has at least 60% sequence identity to SEQ ID NO: 6. In some embodiments, the nucleotide sequence of PSA encoded by the heterologous ORF described herein has at least 65% sequence identity to SEQ ID NO: 6. In some embodiments, the nucleotide sequence of PSA encoded by the heterologous ORF described herein has at least 70% sequence identity to SEQ ID NO: 6. In some embodiments, the nucleotide sequence of PSA encoded by the heterologous ORF described herein has at least 75% sequence identity to SEQ ID NO: 6. In some embodiments, the nucleotide sequence of PSA encoded by the heterologous ORF described herein has at least 80% sequence identity to SEQ ID NO: 6. In some embodiments, the nucleotide sequence of PSA encoded by the heterologous ORF described herein has at least 85% sequence identity to SEQ ID NO: 6. In some embodiments, the nucleotide sequence of PSA encoded by the heterologous ORF described herein has at least 90% sequence identity to SEQ ID NO: 6. In some embodiments, the nucleotide sequence of PSA encoded by the heterologous ORF described herein has at least 91% sequence identity to SEQ ID NO: 6. In some embodiments, the nucleotide sequence of PSA encoded by the heterologous ORF described herein has at least 92% sequence identity to SEQ ID NO: 6. In some embodiments, the nucleotide sequence of PSA encoded by the heterologous ORF described herein has at least 93% sequence identity to SEQ ID NO: 6. In some embodiments, the nucleotide sequence of PSA encoded by the heterologous ORF described herein has at least 94% sequence identity to SEQ ID NO: 6. In some embodiments, the nucleotide sequence of PSA encoded by the heterologous ORF described herein has at least 95% sequence identity to SEQ ID NO: 6. In some embodiments, the nucleotide sequence of PSA encoded by the heterologous ORF described herein has at least 96% sequence identity to SEQ ID NO: 6. In some embodiments, the nucleotide sequence of PSA encoded by the heterologous ORF described herein has at least 97% sequence identity to SEQ ID NO: 6. In some embodiments, the nucleotide sequence of PSA encoded by the heterologous ORF described herein has at least 98% sequence identity to SEQ ID NO: 6. In some embodiments, the nucleotide sequence of PSA encoded by the heterologous ORF described herein has at least 99% sequence identity to SEQ ID NO: 6. In some embodiments, the nucleotide sequence of PSA encoded by the heterologous ORF described herein consists of SEQ ID NO: 6. In some embodiments, the nucleotide sequence of PSA encoded by the heterologous ORF described herein comprises SEQ ID NO: 6.

In some embodiments, the nucleotide sequence of the antigenic fragment of PSMA encoded by the heterologous ORF described herein has at least 50% sequence identity to SEQ ID NO: 7 or 8. In some embodiments, the nucleotide sequence of the antigenic fragment of PSMA encoded by the heterologous ORF described herein has at least 55% sequence identity to SEQ ID NO: 7 or 8. In some embodiments, the nucleotide sequence of the antigenic fragment of PSMA encoded by the heterologous ORF described herein has at least 60% sequence identity to SEQ ID NO: 7 or 8. In some embodiments, the nucleotide sequence of the antigenic fragment of PSMA encoded by the heterologous ORF described herein has at least 65% sequence identity to SEQ ID NO: 7 or 8. In some embodiments, the nucleotide sequence of the antigenic fragment of PSMA encoded by the heterologous ORF described herein has at least 70% sequence identity to SEQ ID NO: 7 or 8. In some embodiments, the nucleotide sequence of the antigenic fragment of PSMA encoded by the heterologous ORF described herein has at least 75% sequence identity to SEQ ID NO: 7 or 8. In some embodiments, the nucleotide sequence of the antigenic fragment of PSMA encoded by the heterologous ORF described herein has at least 80% sequence identity to SEQ ID NO: 7 or 8. In some embodiments, the nucleotide sequence of the antigenic fragment of PSMA encoded by the heterologous ORF described herein has at least 85% sequence identity to SEQ ID NO: 7 or 8. In some embodiments, the nucleotide sequence of the antigenic fragment of PSMA encoded by the heterologous ORF described herein has at least 90% sequence identity to SEQ ID NO: 7 or 8. In some embodiments, the nucleotide sequence of the antigenic fragment of PSMA encoded by the heterologous ORF described herein has at least 91% sequence identity to SEQ ID NO: 7 or 8. In some embodiments, the nucleotide sequence of the antigenic fragment of PSMA encoded by the heterologous ORF described herein has at least 92% sequence identity to SEQ ID NO: 7 or 8. In some embodiments, the nucleotide sequence of the antigenic fragment of PSMA encoded by the heterologous ORF described herein has at least 93% sequence identity to SEQ ID NO: 7 or 8. In some embodiments, the nucleotide sequence of the antigenic fragment of PSMA encoded by the heterologous ORF described herein has at least 94% sequence identity to SEQ ID NO: 7 or 8. In some embodiments, the nucleotide sequence of the antigenic fragment of PSMA encoded by the heterologous ORF described herein has at least 95% sequence identity to SEQ ID NO: 7 or 8. In some embodiments, the nucleotide sequence of the antigenic fragment of PSMA encoded by the heterologous ORF described herein has at least 96% sequence identity to SEQ ID NO: 7 or 8. In some embodiments, the nucleotide sequence of the antigenic fragment of PSMA encoded by the heterologous ORF described herein has at least 97% sequence identity to SEQ ID NO: 7 or 8. In some embodiments, the nucleotide sequence of the antigenic fragment of PSMA encoded by the heterologous ORF described herein has at least 98% sequence identity to SEQ ID NO: 7 or 8. In some embodiments, the nucleotide sequence of the antigenic fragment of PSMA encoded by the heterologous ORF described herein has at least 99% sequence identity to SEQ ID NO: 7 or 8. In some embodiments, the nucleotide sequence of the antigenic fragment of PSMA encoded by the heterologous ORF described herein consists of SEQ ID NO: 7 or 8. In some embodiments, the nucleotide sequence of the antigenic fragment of PSMA encoded by the heterologous ORF described herein comprises SEQ ID NO: 7 or 8.

In other embodiments, provided herein is an arenavirus L segment that is engineered to carry a heterologous ORF encoding a prostate cancer-related antigen or an antigenic fragment thereof as described in the preceeding paragraphs, in addition to an arenavirus ORF encoding an arenavirus protein, such as the GP, NP, Z or L protein.

The arenavirus genome segment provided herein can be derived from any species of arenavirus. In certain embodiments, the arenavirus genome segment provided herein can be derived from Lymphocytic choriomeningitis virus (LCMV). In certain embodiments, the arenavirus genome segment provided herein can be derived from Lassa virus. In certain embodiments, the arenavirus genome segment provided herein can be derived from Pichinde virus. In certain embodiments, the arenavirus genome segment provided herein can be derived from Junin virus. In certain embodiments, the arenavirus genome segment provided herein can be derived from Oliveros virus. In certain embodiments, the arenavirus genome segment provided herein can be derived from Tamiami virus. In certain embodiments, the arenavirus genome segment provided herein can be derived from Mobala virus. In certain embodiments, the arenavirus genome segment provided herein can be derived from Mopeia virus. In certain embodiments, the arenavirus genome segment provided herein can be derived from Ippy virus. In certain embodiments, the arenavirus genome segment provided herein can be derived from Amapari virus. In certain embodiments, the arenavirus genome segment provided herein can be derived from Flexal virus. In certain embodiments, the arenavirus genome segment provided herein can be derived from Guanarito virus. In certain embodiments, the arenavirus genome segment provided herein can be derived from Latino virus. In certain embodiments, the arenavirus genome segment provided herein can be derived from Machupo virus. In certain embodiments, the arenavirus genome segment provided herein can be derived from Parana virus. In certain embodiments, the arenavirus genome segment provided herein can be derived from Pirital virus. In certain embodiments, the arenavirus genome segment provided herein can be derived from Sabia virus. In certain embodiments, the arenavirus genome segment provided herein can be derived from Tacaribe virus. In certain embodiments, the arenavirus genome segment provided herein can be derived from Bear Canyon virus. In certain embodiments, the arenavirus genome segment provided herein can be derived from Whitewater Arroyo virus. In certain embodiments, the arenavirus genome segment provided herein can be derived from Allpahuayo virus (ALLV). In certain embodiments, the arenavirus genome segment provided herein can be derived from Alxa virus. In certain embodiments, the arenavirus genome segment provided herein can be derived from Chapare virus. In certain embodiments, the arenavirus genome segment provided herein can be derived from Lijiang virus. In certain embodiments, the arenavirus genome segment provided herein can be derived from Cupixi virus. In certain embodiments, the arenavirus genome segment provided herein can be derived from Gairo virus. In certain embodiments, the arenavirus genome segment provided herein can be derived from Loei River virus. In certain embodiments, the arenavirus genome segment provided herein can be derived from Lujo virus. In certain embodiments, the arenavirus genome segment provided herein can be derived from Luna virus. In certain embodiments, the arenavirus genome segment provided herein can be derived from Luli virus. In certain embodiments, the arenavirus genome segment provided herein can be derived from Lunk virus. In certain embodiments, the arenavirus genome segment provided herein can be derived from Mariental virus. In certain embodiments, the arenavirus genome segment provided herein can be derived from Merino Walk virus. In certain embodiments, the arenavirus genome segment provided herein can be derived from Morogoro virus. In certain embodiments, the arenavirus genome segment provided herein can be derived from Okahandja virus. In certain embodiments, the arenavirus genome segment provided herein can be derived from Apore virus. In certain embodiments, the arenavirus genome segment provided herein can be derived from Ryukyu virus. In certain embodiments, the arenavirus genome segment provided herein can be derived from Solwezi virus. In certain embodiments, the arenavirus genome segment provided herein can be derived from souris virus. In certain embodiments, the arenavirus genome segment provided herein can be derived from Wenzhou virus. In certain embodiments, the arenavirus genome segment provided herein can be derived from Big Brushy Tank virus. In certain embodiments, the arenavirus genome segment provided herein can be derived from Catarina virus. In certain embodiments, the arenavirus genome segment provided herein can be derived from Skinner Tank virus. In certain embodiments, the arenavirus genome segment provided herein can be derived from Tonto Creek virus. In certain embodiments, the arenavirus genome segment provided herein can be derived from Xapuri virus.

The methods of generating an arenavirus genome segment provided herein follow standard protocols well known in the art (see e.g., Kallert et al., 2017, Nat Commun, 8: 15327).

### 5.2 Nucleic Acids, Vector Systems and Host Cells

Provided herein are cDNAs comprising or consisting of the arenavirus S segment as described in Section 5.1 to form tri-segmented arenavirus particles as described in Section 5.3. Also provided herein are DNA expression vectors comprising the cDNA described in this section. Further provided herein are host cells comprising such cDNAs or vectors described in this section.

### 5.2.1 Nucleic Acids, Vector Systems and Host Cells for an Arenavirus S Segment

In specific embodiments, provided herein is a cDNA of the arenavirus S segment engineered to carry a heterologous ORF encoding a prostate cancer-related antigen that is described in Section 5.1. In other embodiments, provided herein is a cDNA that is an arenavirus L segment that has been engineered to carry a heterologous ORF encoding a prostate cancer-related antigen that is described in Section 5.1. In certain embodiments, provided herein is a cDNA of the arenavirus segments that have been engineered to carry (i) a heterologous ORF encoding prostate cancer-related antigens; and (ii) an ORF encoding an arenavirus GP, NP, Z protein, or L protein, wherein one of the ORFs encoding an arenavirus GP, NP, Z protein, or L protein has been removed and replaced with the heterologous ORF as described in Section 5.1.

In one embodiment, provided herein is a DNA expression vector that encodes an arenavirus S segment engineered to carry a heterologous ORF encoding a prostate cancer-related antigen as described herein. In another embodiment, a cDNA that is an arenavirus S segment that has been engineered to carry a heterologous ORF encoding a prostate cancer-related antigen as described herein is part of or incorporated into a DNA expression vector. In other embodiments, provided herein is a DNA expression vector that encodes an arenavirus L segment that has been engineered to carry a heterologous ORF encoding a prostate cancer-related antigen as described herein. In another embodiment, a cDNA that is an arenavirus L segment that has been engineered to carry a heterologous ORF encoding a prostate cancer-related antigen as described herein is part of or incorporated into a DNA expression vector.

In another embodiment, provided herein is a cell, wherein the cell comprises a cDNA or a vector system described above in this section. Cell lines derived from such cells, cultures comprising such cells, methods of culturing such cells are also provided herein. In certain embodiments, provided herein is a cell, wherein the cell comprises a cDNA of the arenavirus S segment that has been engineered to carry a heterologous ORF encoding a prostate cancer-related antigen as described herein. In some embodiments, the cell comprises the S segment and/or the L segment.

### 5.2.2 Nucleic Acids, Vector Systems and Host Cells for a Tri-Segmented Arenavirus Particle

Provided herein are nucleic acids that encode the three arenavirus genomic segments of a tri-segmented arenavirus particle as described in Section 5.3. In more specific embodiments, provided herein is a DNA nucleotide sequence or a set of DNA nucleotide sequences, for example, as set forth in Table 1. Host cells that comprise such nucleic acids are also provided.

In specific embodiments, provided herein is a series of DNA expression vectors that together encode the tri-segmented arenavirus particle as described in Section 5.3. Specifically, provided herein is a series of DNA expression vectors encoding three arenavirus genomic segments, namely, one L segment and two S segments of a tri-segmented arenavirus particle as described herein.

In another embodiment, provided herein is a cell, wherein the cell comprises a series of vectors described above in this section. Cell lines derived from such cells, cultures comprising such cells, methods of culturing such cells are also provided herein.

### 5.3 Tri-Segmented Arenavirus Particles

Provided herein is a tri-segmented arenavirus particle comprising one arenavirus L segment and two arenavirus S segments, wherein the two arenavirus S segments are as described in Section 5.1, and wherein one of the two arenavirus S segments comprises GP and the other comprises NP. Also provided herein is a tri-segmented arenavirus particle comprising one arenavirus L segment and two arenavirus S segments, wherein the two ORFs encoding a prostate cancer-related antigen as described in Section 5.1 are inserted into two of the three segments.

Table 1, below, is an exemplary illustration of the genome organization of a tri-segmented arenavirus particle comprising one L segment and two S segments, wherein intersegmental recombination of the two S segments in the tri-segmented arenavirus genome does not result in a replication-competent bi-segmented viral particle and abrogates arenaviral promoter activity (i.e., the resulting recombined S segment is made up of two 3′UTRs or two 5′ UTRs instead of a 3′ UTR and a 5′ UTR).

TABLE 1
Tri-segmented arenavirus particle comprising
one L segment and two S segments

| Position 1 | Position 2 | Position 3 | Position 4 | Position 5 | Position 6 |
|---|---|---|---|---|---|
| *ORF | GP | *ORF | NP | Z | L |
| *ORF | NP | *ORF | GP | Z | L |
| *ORF | GP | *ORF | NP | L | Z |
| *ORF | NP | *ORF | GP | L | Z |
| GP | *ORF | NP | *ORF | Z | L |
| NP | *ORF | GP | *ORF | Z | L |
| GP | *ORF | NP | *ORF | L | Z |
| NP | *ORF | GP | *ORF | L | Z |

Position 1 is under the control of a first arenavirus S segment 5′ UTR; Position 2 is under the control of a first arenavirus S segment 3′ UTR; Position 3 is under the control of a second arenavirus S segment 5′ UTR; Position 4 is under the control of a second arenavirus S segment 3′ UTR; Position 5 is under the control of an arenavirus L segment 5′ UTR;Position 6 is under the control of an arenavirus L segment 3′ UTR.
*ORF indicates a heterologous ORF encoding a prostate cancer-related antigen or an antigenic fragment thereof as described in Section 5.1.

In certain embodiments, the Intergenic region (IGR) between position one and position two can be an arenavirus S segment or L segment IGR; the IGR between position three and four can be an arenavirus S segment or L segment IGR; and the IGR between the position five and six can be an arenavirus L segment IGR. In a specific embodiment, the IGR between position one and position two can be an arenavirus S segment IGR; the IGR between position three and four can be an arenavirus S segment IGR; and the IGR between the position five and six can be an arenavirus L segment IGR. In certain embodiments, other combinations are also possible. In certain embodiments, intersegmental recombination of the two S segments in the tri-segmented arenavirus genome of a tri-segmented arenavirus particle comprising one L segment and two S segments does not result in a replication-competent bi-segmented viral particle and abrogates arenaviral promoter activity (i.e., the resulting recombined S segment is made up of two 3′UTRs or two 5′UTRs instead of a 3′ UTR and a 5′ UTR).

In certain embodiments, intersegmental recombination of an S segment and an L segment in the tri-segmented arenavirus particle comprising one L segment and two S segments, restores a functional segment with two viral genes on only one segment instead of two separate segments. In other embodiments, intersegmental recombination of an S segment and an L segment in the tri-segmented arenavirus particle comprising one L segment and two S segments does not result in a replication-competent bi-segmented viral particle.

In certain embodiments, one of skill in the art could construct an arenavirus genome with an organization as illustrated in Table 1 and as described herein, and then use an assay as described in Section 5.7 to determine whether the tri-segmented arenavirus particle is genetically stable, i.e., does not result in a replication-competent bi-segmented viral particle as discussed herein.

In addition to not resulting in a replication-competent bi-segmented viral particle as described above, in some embodiments, the tri-segmented arenavirus particle has a stable expression of the prostate cancer-related antigen or an antigenic fragment thereof as described herein after being passaged multiple generations, which is necessary for larger-scale commercial production. Therefore, provided herein is a tri-segmented arenavirus particle that has stable expression of the prostate cancer-related antigen or an antigenic fragment thereof as described herein after being passaged at least 4 generations. In other embodiments, provided herein is a tri-segmented arenavirus particle that has stable expression of the prostate cancer-related antigen or an antigenic fragment thereof as described herein after being passaged at least 5 generations. In other embodiments, provided herein is a tri-segmented arenavirus particle that has stable expression of the prostate cancer-related antigen or an antigenic fragment thereof as described herein after being passaged at least 6 generations. In other embodiments, provided herein is a tri-segmented arenavirus particle that has stable expression of the prostate cancer-related antigen or an antigenic fragment thereof as described herein after being passaged at least 7 generations. In other embodiments, provided herein is a tri-segmented arenavirus particle that has stable expression of the prostate cancer-related antigen or an antigenic fragment thereof as described herein after being passaged at least 8 generations. In other embodiments, provided herein is a tri-segmented arenavirus particle that has stable expression of the prostate cancer-related antigen or an antigenic fragment thereof as described herein after being passaged at least 9 generations. In other embodiments, provided herein is a tri-segmented arenavirus particle that has stable expression of the prostate cancer-related antigen or an antigenic fragment thereof as described herein after being passaged at least 10 generations.

As demonstrated in Example sections 6.1.1 and 6.1.2, provided herein is a tri-segmented arenavirus particle comprising one arenavirus L segment and two arenavirus S segments, wherein a first arenavirus S segment is engineered to carry a heterologous ORF consisting of SEQ ID NO: 5 in a position under control of an arenavirus 5′ UTR and an ORF encoding viral nucleoprotein (NP) in a position under control of an arenavirus 3′ UTR, and a second arenavirus S segment is engineered to carry a heterologous ORF consisting of SEQ ID NO: 6 in a position under control of an arenavirus 5′ UTR and an ORF encoding viral glycoprotein (GP) in a position under control of an arenavirus 3′ UTR.

In a specific embodiment, provided herein is a tri-segmented arenavirus particle comprising two S segments, wherein one of the two S segments comprises SEQ ID NO. 10, and the other one of the two S segments comprises SEQ ID NO. 11

In a specific embodiment, provided herein is a tri-segmented arenavirus particle comprising two S segments, wherein one of the two S segments comprises SEQ ID NO. 12, and the other one of the two S segments comprises SEQ ID NO. 13.

As demonstrated in Example sections 6.1.3, provided herein is a tri-segmented arenavirus particle comprising one arenavirus L segment and two arenavirus S segments, wherein a first arenavirus S segment is engineered to carry a heterologous ORF consisting of SEQ ID NO: 8 in a position under control of an arenavirus 5′ UTR and an ORF encoding viral nucleoprotein (NP) in a position under control of an arenavirus 3′ UTR, and a second arenavirus S segment is engineered to carry a heterologous ORF consisting of SEQ ID NO: 7 in a position under control of an arenavirus 5′ UTR and an ORF encoding viral glycoprotein (GP) in a position under control of an arenavirus 3′ UTR.

In a specific embodiment, provided herein is a tri-segmented arenavirus particle comprising two S segments, wherein one of the two S segments comprises SEQ ID NO. 14, and the other one of the two S segments comprises SEQ ID NO. 15.

As demonstrated in Example sections 6.1.4, provided herein is a tri-segmented arenavirus particle comprising one arenavirus L segment and two arenavirus S segments, wherein a first arenavirus S segment is engineered to carry a heterologous ORF consisting of SEQ ID NO: 7 in a position under control of an arenavirus 5′ UTR and an ORF encoding viral nucleoprotein (NP) in a position under control of an arenavirus 3′ UTR, and a second arenavirus S segment is engineered to carry a heterologous ORF consisting of SEQ ID NO: 8 in a position under control of an arenavirus 5′ UTR and an ORF encoding viral glycoprotein (GP) in a position under control of an arenavirus 3′ UTR.

In a specific embodiment, provided herein is a tri-segmented arenavirus particle comprising two S segments, wherein one of the two S segments comprises SEQ ID NO. 16, and the other one of the two S segments comprises SEQ ID NO. 17

In certain embodiments, the tri-segmented arenavirus particle as provided herein is infectious, i.e., is capable of entering into or injecting its genetic material into a host cell. In certain more specific embodiments, the tri-segmented arenavirus particle as provided herein is infectious, i.e., is capable of entering into or injecting its genetic material into a host cell followed by amplification and expression of its genetic information inside the host cell. In certain embodiments, the tri-segmented arenavirus particle is an infectious, replication-deficient arenavirus particle engineered to contain a genome with the ability to amplify and express its genetic information in infected cells but unable to produce further infectious progeny particles in normal, not genetically engineered cells. In certain embodiments, the infectious tri-segmented arenavirus particle is replication-competent and able to produce further infectious progeny particles in normal, not genetically engineered cells. In certain more specific embodiments, such a replication-competent viral vector is attenuated relative to the wild type virus from which the replication-competent viral vector is derived.

In certain embodiments, the arenavirus particle is derived from a Lassa virus. In certain embodiments, the arenavirus particle is derived from a Lymphocytic choriomeningitis virus (LCMV). In certain embodiments, the LCMV is Clone 13, MP strain, Arm CA 1371, Arm E-250, WE, LCMV c113/WE (i.e. LCMV clone 13 expressing the glycoprotein of LCMV strain WE instead of endogenous LCMV clone 13 glycoprotein), UBC, Traub, Pasteur, 810885, CH-5692, Marseille #12, HP65-2009, 200501927, 810362, 811316, 810316, 810366, 20112714, Douglas, GR01, SN05, CABN and their derivatives. In certain embodiments, the arenavirus particle is derived from a Pichinde virus (PICV). In certain embodiments, the PICV is strain Munchique CoAn4763 isolate P18, P2 strain, or is derived from any of the several isolates described by Trapido and colleagues (Trapido et al, 1971, Am J Trop Med Hyg, 20: 631-641). In certain embodiments, the arenavirus particle is derived from a Junin virus vaccine Candid #1, or a Junin virus vaccine XJ Clone 3 strain. In certain embodiments, the arenavirus particle is derived from an Oliveros virus. In certain embodiments, the arenavirus particle is derived from a Tamiami virus. In certain embodiments, the arenavirus particle is derived from a Mobala virus. In certain embodiments, the arenavirus particle is derived from a Mopeia virus. In certain embodiments, the arenavirus particle is derived from an Ippy virus. In certain embodiments, the arenavirus particle is derived from an Amapari virus. In certain embodiments, the arenavirus particle is derived from a Flexal virus. In certain embodiments, the arenavirus particle is derived from a Guanarito virus. In certain embodiments, the arenavirus particle is derived from a Latino virus. In certain embodiments, the arenavirus particle is derived from a Machupo virus. In certain embodiments, the arenavirus particle is derived from a Parana virus. In certain embodiments, the arenavirus particle is derived from a Pirital virus. In certain embodiments, the arenavirus particle is derived from a Sabia virus. In certain embodiments, the arenavirus particle is derived from a Tacaribe virus. In certain embodiments, the arenavirus particle is derived from a Bear Canyon virus. In certain embodiments, the arenavirus particle is derived from a Whitewater Arroyo virus. In certain embodiments, the arenavirus particle is derived from a Allpahuayo virus (ALLV). In certain embodiments, the arenavirus particle is derived from an Alxa virus. In certain embodiments, the arenavirus particle is derived from a Chapare virus. In certain embodiments, the arenavirus particle is derived from a Lijiang virus. In certain embodiments, the arenavirus particle is derived from a Cupixi virus. In certain embodiments, the arenavirus particle is derived from a Gairo virus. In certain embodiments, the arenavirus particle is derived from a Loei River virus. In certain embodiments, the arenavirus particle is derived from a Lujo virus. In certain embodiments, the arenavirus particle is derived from a Luna virus. In certain embodiments, the arenavirus particle is derived from a Luli virus. In certain embodiments, the arenavirus particle is derived from a Lunk virus. In certain embodiments, the arenavirus particle is derived from a Mariental virus. In certain embodiments, the arenavirus particle is derived from a Merino Walk virus. In certain embodiments, the arenavirus particle is derived from a Morogoro virus. In certain embodiments, the arenavirus particle is derived from an Okahandja virus. In certain embodiments, the arenavirus particle is derived from an Apore virus. In certain embodiments, the arenavirus particle is derived from a Ryukyu virus. In certain embodiments, the arenavirus particle is derived from a Solwezi virus. In certain embodiments, the arenavirus particle is derived from a souris virus. In certain embodiments, the arenavirus particle is derived from a Wenzhou virus. In certain embodiments, the arenavirus particle is derived from a Big Brushy Tank virus. In certain embodiments, the arenavirus particle is derived from a Catarina virus. In certain embodiments, the arenavirus particle is derived from a Skinner Tank virus. In certain embodiments, the arenavirus particle is derived from a Tonto Creek virus. In certain embodiments, the arenavirus particle is derived from a Xapuri virus.

In certain embodiments, provided herein is a replication deficient arenavirus particle in which (i) one or more of its genome segment(s) are engineered to carry a heterologous ORF encoding a prostate cancer-related antigen or an antigenic fragment thereof as described herein; and (ii) an ORF encoding GP, NP, Z protein, or L protein has been removed or functionally inactivated such that the resulting virus cannot produce further infectious progeny virus particles. An arenavirus particle comprising a genetically modified genome in which one or more ORFs has been deleted or functionally inactivated can be produced in complementing cells (i.e., cells that express the arenavirus ORF that has been deleted or functionally inactivated) (see, e.g., WO 2009083210, which is incorporated herein by reference in its entirety).

In certain embodiments, provided herein is a replication-competent arenavirus particle in which: (i) one or more of its genome segment(s) are engineered to carry a heterologous ORF encoding a prostate cancer-related antigen or an antigenic fragment thereof as described herein; and (ii) ORFs encoding GP, NP, Z protein, and L protein are expressed, but one or more of these ORFs encoding GP, NP, Z protein, and L protein are in a position under the control of a UTR other than the wild-type UTR for the corresponding ORF (see, e.g., WO 2016075250, which is incorporated herein by reference in its entirety).

In certain embodiments, the present application relates to the arenavirus particle as described in the preceding paragraph suitable for use as a vaccine and methods of using such arenavirus particle in a vaccination and treatment of prostate cancer. In certain embodiments, provided herein is a kit comprising, in one or more containers, one or more cDNAs as described in Section 5.2. In a specific embodiment, a kit comprises, in one or two or more containers an arenavirus S segment or an arenavirus particle as described in the preceding paragraphs. The kit may further comprise one or more of the following: a host cell suitable for rescue of the arenavirus S segment or the arenavirus particle, reagents suitable for transfecting plasmid cDNA into a host cell, a helper virus, plasmids encoding viral proteins and/or one or more primers specific for a modified arenavirus S segment or arenavirus particle or cDNAs of the same.

### 5.4 Methods of Generating a Tri-Segmented Arenavirus Particle

A tri-segmented arenavirus particle can be recombinantly produced by reverse genetic techniques known in the art, for example as described by Emonet et al., 2008, PNAS, 106(9):3473-3478; Popkin et al., 2011, J. Virol., 85 (15):7928-7932, WO2016075250, WO2016198531, WO2017076988, WO2017080920, WO2017198726, WO2018083220 and WO2018185307, which are incorporated by reference herein.

### 5.4.1 Infectious and Replication Competent Tri-Segmented Arenavirus Particle

In certain embodiments, the method of generating the tri-segmented arenavirus particle as described in Section 5.3 comprises (i) transfecting into a host cell the nucleic acids of one L segment and two S segments; (ii) maintaining the host cell under conditions suitable for virus formation; and (iii) harvesting the cell culture supernatant containing the arenavirus particle.

Once generated from cDNA, the tri-segmented arenavirus particle as described herein (i.e., infectious and replication competent) can be propagated. In certain embodiments, the tri-segmented arenavirus particle can be propagated in any host cell that allows the virus to grow to titers that permit the uses of the virus as described herein. In one embodiment, the host cell allows the tri-segmented arenavirus particle as described herein to grow to titers comparable to those determined for the corresponding wild-type virus.

In certain embodiments, the tri-segmented arenavirus particle as described herein may be propagated in host cells. Specific examples of host cells that can be used include BHK, HEK 293, VERO cells or other. In a specific embodiment, the tri-segmented arenavirus particle as described herein may be propagated in a cell line.

In certain embodiments, the host cells are kept in culture and are transfected with one or more plasmid(s). The plasmid(s) encode the arenavirus genomic segment(s) to be expressed from one or more expression cassette(s) suitable for expression in mammalian cells, e.g., comprising a polymerase I promoter and terminator.

In specific embodiments, the host cells are kept in culture and are transfected with one or more plasmid(s). The plasmid(s) encode the viral gene(s) to be generated expressed from one or more expression cassette(s) suitable for expression in mammalian cells, e.g., comprising a polymerase I promoter and terminator.

Plasmids that can be used for generating the tri-segmented arenavirus particle comprising one L segment and two S segments can include: i) two plasmids each encoding the S genome segment e.g., pol-I S, ii) a plasmid encoding the L genome segment e.g., pol-I L.

In certain embodiments, plasmids encoding an arenavirus polymerase that direct intracellular synthesis of the viral L and S segments can be incorporated into the transfection mixture. For example, a plasmid encoding the L protein and a plasmid encoding NP. The L protein and NP are the minimal trans-acting factors for viral RNA transcription and replication. Alternatively, intracellular synthesis of viral L and S segments, together with NP and L protein can be performed using a bidirectional expression cassette with pol-I and pol-II promoters reading from opposite sides into the L and S segment cDNAs of two separate plasmids, respectively.

In addition, the plasmid(s) features a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Transfection of BHK-21 or HEK 293 cells with a plasmid(s) can be performed using any of the commonly used strategies such as calcium-phosphate, liposome-based protocols or electroporation. A few days after transfection the suitable selection agent, e.g., puromycin, is added in titrated concentrations. Surviving clones are isolated and subcloned following standard procedures, and high-expressing clones are identified using Western blot or flow cytometry procedures with antibodies directed against the viral protein(s) of interest.

Typically, RNA polymerase I-driven expression cassettes, RNA polymerase II-driven cassettes or T7 bacteriophage RNA polymerase driven cassettes can be used, the latter preferentially with a 3′-terminal ribozyme for processing of the primary transcript to yield the correct end. In certain embodiments, the plasmids encoding the arenavirus genomic segments can be the same, i.e., the genome sequence and transacting factors can be transcribed by T7, polI and polII promoters from one plasmid.

For recovering the tri-segmented arenavirus particle, the following procedures are envisaged. First day: cells, typically 80% confluent in M6-well plates, are transfected with a mixture of the plasmids, as described above. For this one can exploit any commonly used strategies such as calcium-phosphate, liposome-based protocols or electroporation. 3-5 days later: The cultured supernatant (arenavirus particle preparation) is harvested, aliquoted and stored at 4° C., −20° C., or −80° C., depending on how long the arenavirus particle should be stored prior use. The arenavirus particle preparation's infectious titer is assessed by an immunofocus assay. Alternatively, the transfected cells and supernatant may be passaged to a larger vessel (e.g., a T75 tissue culture flask) on day 3-5 after transfection, and culture supernatant is harvested up to five days after passage.

The present application furthermore relates to expression of a prostate cancer-related antigen or an antigenic fragment thereof as described herein. The ORF of prostate cancer-related antigen or an antigenic fragment thereof as described herein can be incorporated into the plasmid using restriction enzymes.

### 5.4.2 Infectious, Replication-Defective Tri-Segmented Arenavirus Particle

Infectious, replication-defective tri-segmented arenavirus particles can be rescued as described above. However, once generated from cDNA, the infectious, replication-deficient arenaviruses provided herein can be propagated in complementing cells. Complementing cells are cells that provide the functionality that has been eliminated from the replication-deficient arenavirus by modification of its genome (e.g., if the ORF encoding the GP protein is deleted or functionally inactivated, a complementing cell does provide the GP protein).

Cells that can be used, e.g., BHK-21, HEK 293, MC57G or other, are kept in culture and are transfected with the complementation plasmid(s) using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing C-cell clones are identified using Western blot or flow cytometry procedures with antibodies directed against the viral protein(s) of interest. As an alternative to the use of stably transfected C-cells transient transfection of normal cells can complement the missing viral gene(s) in each of the steps where C-cells will be used. In addition, a helper virus can be used to provide the missing functionality in trans.

### 5.5 Pharmaceutical Compositions

The present application furthermore relates to vaccines, immunogenic compositions (e.g., vaccine formulations), and pharmaceutical compositions comprising a tri-segmented arenavirus particle as described herein. Such vaccines, immunogenic compositions and pharmaceutical compositions can be formulated according to standard procedures in the art. It will be readily apparent to one of ordinary skill in the relevant arts that suitable modifications and adaptations to the methods and applications described herein can be obvious and can be made without departing from the scope or any embodiment thereof.

In certain embodiments, provided herein are immunogenic compositions comprising an arenavirus particle as described herein. In certain embodiments, such an immunogenic composition further comprises a pharmaceutically acceptable excipient. In certain embodiments, such an immunogenic composition further comprises an adjuvant. The adjuvant for administration in combination with a composition described herein may be administered before, concomitantly with, or after administration of said composition. In some embodiments, the term “adjuvant” refers to a compound that when administered in conjunction with or as part of a composition described herein augments, enhances and/or boosts the immune response to a tri-segmented arenavirus particle and, most importantly, the gene products it vectorises, but when the compound is administered alone does not generate an immune response to the tri-segmented arenavirus particle as described herein and the gene products vectorised by the latter. In some embodiments, the adjuvant generates an immune response to the tri-segmented arenavirus particle as described herein and the gene products vectorised by the latter and does not produce an allergy or other adverse reaction. Adjuvants can enhance an immune response by several mechanisms including, e.g., lymphocyte recruitment, stimulation of B and/or T cells, and stimulation of macrophages or dendritic cells. When a vaccine or immunogenic composition comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., 1995, in Vaccine Design. The Subunit and Adjuvant Approach (eds. Powell & Newman, Plenum Press, NY); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., 1997, N. Engl. J. Med. 336, 86-91).

The compositions comprise the tri-segmented arenavirus particle described herein alone or together with a pharmaceutically acceptable carrier. Suspensions or dispersions of the tri-segmented arenavirus particle as described herein, especially isotonic aqueous suspensions or dispersions, can be used. The pharmaceutical compositions may be sterilized and/or may comprise excipients, e.g., preservatives, stabilizers, wetting agents and/or emulsifiers, solubilizers, salts for regulating osmotic pressure and/or buffers and are prepared in a manner known per se, for example by means of conventional dispersing and suspending processes. In certain embodiments, such dispersions or suspensions may comprise viscosity-regulating agents. The suspensions or dispersions are kept at temperatures around 2° C. to 8° C., or preferentially for longer storage may be frozen and then thawed shortly before use, or alternatively may be lyophilized for storage. For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

In certain embodiments, the compositions described herein additionally comprise a preservative, e.g., the mercury derivative thimerosal. In a specific embodiment, the pharmaceutical compositions described herein comprise 0.001% to 0.01% thimerosal. In other embodiments, the pharmaceutical compositions described herein do not comprise a preservative.

In another embodiment, provided herein are compositions comprising a tri-segmented arenavirus particle described herein. Such compositions can be used in methods of treatment and prevention of prostate cancer. In other embodiments, the compositions described herein are used in the treatment of subjects susceptible to or exhibiting symptoms characteristic of prostate cancer or are diagnosed with prostate cancer. In another specific embodiment, the immunogenic compositions provided herein can be used to induce an immune response in a host to whom the composition is administered. The immunogenic compositions described herein can be used as vaccines and can accordingly be formulated as pharmaceutical compositions. In a specific embodiment, the immunogenic compositions described herein are used in the prevention of prostate cancer of subjects (e.g., human subjects). In other embodiments, the vaccine, immunogenic composition or pharmaceutical composition are suitable for veterinary and/or human administration.

### 5.6 Methods of Treating Prostate Cancer

### 5.6.1 Treatment of Prostate Cancer

The tri-segmented arenavirus particle comprising the prostate cancer-related antigens or antigenc fragments thereof described in Section 5.3 and the pharmaceutical composition described in Section 5.5 are designed to induce a potent T cell response directed against prostate tumor cells expressing the same antigens. In some embodiments, the tri-segmented arenavirus particle described in Section 5.3 targets DCs and macrophages, thus delivering antigens for efficient cytotoxic T lymphocyte (CTL) induction. As a result, influx of the induced prostate cancer-related antigen-specific effector CD8+ T cells into the tumor results in CTL-mediated elimination of prostate cancer cells.

Accordingly, in some embodiments, provided herein is a method of treating prostate cancer comprising administering to a subject in need thereof the pharmaceutical composition as described in Section 5.5 in a therapeutically effective amount. In other embodiments, provided herein is a method of preventing prostate cancer comprising administering to a subject in need thereof the pharmaceutical composition as described in Section 5.5 in a therapeutically effective amount.

In one embodiment, administration of the pharmaceutical composition is parenteral administration. Parenteral administration can be intravenous or subcutaneous administration. In another embodiment, the arenaviral particle or the pharmaceutical composition provided herein is administered to a subject by, including but not limited to, oral, intradermal, intramuscular, intraperitoneal, intravenous, topical, subcutaneous, percutaneous, intranasal and inhalation routes, via scarification (scratching through the top layers of skin, e.g., using a bifurcated needle), and via intratumoral administration.

For administration intranasally or by inhalation, the preparation for use according to the present disclosure can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflators may be formulated containing a powder mix of the compound and as suitable powder base such as lactose or starch.

### 5.6.2 Alternating Vector Therapy

In some embodiments, provided herein is a method for treating prostate cancer in a subject in need thereof, wherein the method comprises (i) administering to the subject a first pharmaceutical composition, wherein the first pharmaceutical composition comprises one or more arenavirus particles as described in Section 5.3; and (ii) administering to the subject, after a period of time, a second pharmaceutical composition, wherein the second pharmaceutical composition comprises one or more arenavirus particles as described in Section 5.3. In other embodiments, provided herein is a method for preventing prostate cancer in a subject in need thereof, wherein the method comprises (i) administering to the subject a first pharmaceutical composition, wherein the first pharmaceutical composition comprises one or more arenavirus particles as described in Section 5.3; and (ii) administering to the subject, after a period of time, a second pharmaceutical composition, wherein the second pharmaceutical composition comprises one or more arenavirus particles as described in Section 5.3.

In some embodiments, provided herein is a method for treating prostate cancer in a subject in need thereof, wherein the method comprises (i) administering to the subject a first pharmaceutical composition, wherein the first pharmaceutical composition comprises one or more arenavirus particles as described in Section 5.3; (ii) administering to the subject, after a period of time, a second pharmaceutical composition, wherein the second pharmaceutical composition comprises one or more arenavirus particles as described in Section 5.3; (iii) administering to the subject, after a period of time, the first pharmaceutical composition again; and (iv) administering to the subject, after a period of time, the second pharmaceutical composition again. In other embodiments, provided herein is a method for preventing prostate cancer in a subject in need thereof, wherein the method comprises (i) administering to the subject a first pharmaceutical composition, wherein the first pharmaceutical composition comprises one or more arenavirus particles as described in Section 5.3; (ii) administering to the subject, after a period of time, a second pharmaceutical composition, wherein the second pharmaceutical composition comprises one or more arenavirus particles as described in Section 5.3; (iii) administering to the subject, after a period of time, the first pharmaceutical composition again; and (iv) administering to the subject, after a period of time, the second pharmaceutical composition again.

In some embodiments, provided herein is a method for treating prostate cancer in a subject in need thereof, wherein the method comprises (i) administering to the subject a first pharmaceutical composition, wherein the first pharmaceutical composition comprises one or more arenavirus particles as described in Section 5.3; (ii) administering to the subject, after a period of time, a second pharmaceutical composition, wherein the second pharmaceutical composition comprises one or more arenavirus particles as described in Section 5.3; (iii) administering to the subject, after a period of time, the first pharmaceutical composition again; (iv) administering to the subject, after a period of time, the second pharmaceutical composition again; (v) administering to the subject, after a period of time, the first pharmaceutical composition again; and (vi) administering to the subject, after a period of time, the second pharmaceutical composition again. In other embodiments, provided herein is a method for preventing prostate cancer in a subject in need thereof, wherein the method comprises (i) administering to the subject a first pharmaceutical composition, wherein the first pharmaceutical composition comprises one or more arenavirus particles as described in Section 5.3; (ii) administering to the subject, after a period of time, a second pharmaceutical composition, wherein the second pharmaceutical composition comprises one or more arenavirus particles as described in Section 5.3; (iii) administering to the subject, after a period of time, the first pharmaceutical composition again; (iv) administering to the subject, after a period of time, the second pharmaceutical composition again; (v) administering to the subject, after a period of time, the first pharmaceutical composition again; and (vi) administering to the subject, after a period of time, the second pharmaceutical composition again.

In some embodiments, provided herein is a method for treating prostate cancer in a subject in need thereof, wherein the method comprises (i) administering to the subject a first pharmaceutical composition, wherein the first pharmaceutical composition comprises one or more arenavirus particles as described in Section 5.3; (ii) administering to the subject, after a period of time, a second pharmaceutical composition, wherein the second pharmaceutical composition comprises one or more arenavirus particles as described in Section 5.3; and repeat (i) and (ii) an extra 3 times, 4 times, 5 times, 6 times, 7 times, 8 times, 9 times, or 10 times. In other embodiments, provided herein is a method for preventing prostate cancer in a subject in need thereof, wherein the method comprises (i) administering to the subject a first pharmaceutical composition, wherein the first pharmaceutical composition comprises one or more arenavirus particles as described in Section 5.3; (ii) administering to the subject, after a period of time, a second pharmaceutical composition, wherein the second pharmaceutical composition comprises one or more arenavirus particles as described in Section 5.3; and repeat (i) and (ii) an extra 3 times, 4 times, 5 times, 6 times, 7 times, 8 times, 9 times, or 10 times.

In some embodiments, the one or more arenavirus particles from the first and the second pharmaceutical compositions in the preceding paragraphs are derived from different arenavirus species, but carry ORF(s) encoding the same prostate cancer-related antigens or antigenic fragments thereof as described herein. In other embodiments, the one or more arenavirus particles from the first and the second pharmaceutical compositions in the preceding paragraphs are derived from different arenavirus species, and carry ORF(s) encoding different prostate cancer-related antigens or antigenic fragments thereof as described herein. In yet other embodiments, the one or more arenavirus particles from the first and the second pharmaceutical compositions in the preceding paragraphs are derived from the same arenavirus species, but carry ORF(s) encoding different prostate cancer-related antigens or antigenic fragments thereof as described herein.

In specific embodiments, the one or more arenavirus particles from the first pharmaceutical composition are derived from PICV, and the one or more arenavirus particles from the second pharmaceutical composition are derived from LCMV, and the arenavirus particles carry ORF(s) encoding the same prostate cancer-related antigens or antigenic fragments thereof as described herein. In other specific embodiments, the one or more arenavirus particles from the first pharmaceutical composition are derived from PICV, and the one or more arenavirus particles from the second pharmaceutical composition are derived from LCMV, and the arenavirus particles carry ORF(s) encoding different prostate cancer-related antigens or antigenic fragments thereof as described herein. In yet other embodiments, the one or more arenavirus particles from the first pharmaceutical composition are derived from LCMV, the one or more arenavirus particles from the second pharmaceutical composition are derived from PICV, and the arenavirus particles carry ORF(s) encoding the same prostate cancer-related antigens or antigenic fragments thereof as described herein. In yet still other embodiments, the one or more arenavirus particles from the first pharmaceutical composition are derived from LCMV, the one or more arenavirus particles from the second pharmaceutical composition are derived from PICV, and the arenavirus particles carry ORF(s) encoding different prostate cancer-related antigens or antigenic fragments thereof as described herein.

In specific embodiments, the one or more arenavirus particles from the first pharmaceutical composition are derived from PICV, and the one or more arenavirus particles from the second pharmaceutical composition are derived from LCMV, and the arenavirus particles from both pharmaceutical compositions carry ORF(s) encoding PAP and PSA. In specific embodiments, the one or more arenavirus particles from the first pharmaceutical composition are derived from PICV, and the one or more arenavirus particles from the second pharmaceutical composition are derived from LCMV, and the arenavirus particles from both pharmaceutical compositions carry ORF(s) encoding the same antigenic fragments of PSMA. In specific embodiments, the one or more arenavirus particles from the first pharmaceutical composition are derived from PICV, and the one or more arenavirus particles from the second pharmaceutical composition are derived from LCMV, and the arenavirus particles from both pharmaceutical compositions carry ORF(s) encoding PAP, PSA, and the same antigenic fragments of PSMA. In other specific embodiments, the one or more arenavirus particles from the first pharmaceutical composition are derived from PICV and carry ORF(s) encoding PAP and PSA, and the one or more arenavirus particles from the second pharmaceutical composition are derived from LCMV and carry ORF(s) encoding antigenic fragments of PSMA. In other specific embodiments, the one or more arenavirus particles from the first pharmaceutical composition are derived from PICV and carry ORF(s) encoding antigenic fragments of PSMA, and the one or more arenavirus particles from the second pharmaceutical composition are derived from LCMV and carry ORF(s) encoding PAP and PSA.

In specific embodiments, the one or more arenavirus particles from the first pharmaceutical composition are derived from LCMV, and the one or more arenavirus particles from the second pharmaceutical composition are derived from PICV, and the arenavirus particles from both pharmaceutical compositions carry ORF(s) encoding PAP and PSA. In specific embodiments, the one or more arenavirus particles from the first pharmaceutical composition are derived from LCMV, and the one or more arenavirus particles from the second pharmaceutical composition are derived from PICV, and the arenavirus particles from both pharmaceutical compositions carry ORF(s) encoding the same antigenic fragments of PSMA. In specific embodiments, the one or more arenavirus particles from the first pharmaceutical composition are derived from LCMV, and the one or more arenavirus particles from the second pharmaceutical composition are derived from PICV, and the arenavirus particles from both pharmaceutical compositions carry ORF(s) encoding PAP, PSA, and the same antigenic fragments of PSMA. In other specific embodiments, the one or more arenavirus particles from the first pharmaceutical composition are derived from LCMV and carry ORF(s) encoding PAP and PSA, and the one or more arenavirus particles from the second pharmaceutical composition are derived from PICV and carry ORF(s) encoding antigenic fragments of PSMA. In other specific embodiments, the one or more arenavirus particles from the first pharmaceutical composition are derived from LCMV and carry ORF(s) encoding antigenic fragments of PSMA, and the one or more arenavirus particles from the second pharmaceutical composition are derived from PICV and carry ORF(s) encoding PAP and PSA.

### 5.6.3 Dosing and Regime

Also provided herein are dosings of the methods for treating prostate cancer with the pharmaceutical compositions described herein. In some embodiments, the pharmaceutical compositions containing the tri-segmented arenavirus particles described herein are administered with about 1×10⁶ replication-competent virus focus forming units (RCV FFU). In other embodiments, the pharmaceutical compositions containing the tri-segmented arenavirus particles described herein are administered with about 1×10⁷ RCV FFU. In other embodiments, the pharmaceutical compositions containing the tri-segmented arenavirus particles described herein are administered with about 1×10⁸ RCV FFU. In other embodiments, the pharmaceutical compositions containing the tri-segmented arenavirus particles described herein are administered with about 1×10⁹ RCV FFU.

Also provided herein are dosings of the methods for treating prostate cancer with the pharmaceutical compositions described herein. As described in Section 5.6.2, pharmaceutical compositions containing the same or different tri-segmented arenavirus particles can be alternated. As such, in some embodiments, the second pharmaceutical composition can be administered about 22 days after the first pharmaceutical composition. In other embodiments, the second pharmaceutical composition can be administered about 43 days after the first pharmaceutical composition.

### 5.6.4 CombinationTherapy

Also provided herein are methods of treating prostate cancer in a subject in need thereof, wherein the method comprises (i) administering to the subject a first pharmaceutical composition, wherein the first pharmaceutical composition comprises one or more arenavirus particles as described in Section 5.3; (ii) administering to the subject, after a period of time, a second pharmaceutical composition, wherein the second pharmaceutical composition comprises one or more arenavirus particles as described in Section 5.3; and (iii) administering a second agent in combination with the first and/or second pharmaceutical composition.

The second agent provided herein is an agent that is well known in the art to treat prostate cancer. In some embodiments, the second agent is a chemotherapy drug that generally inhibits growth of tumor cells. In other embodiments, the second agent is a targeted therapy drug for mutated gene(s) that are associated with prostate cancer. In other embodiments, the second agent is an androgen axis inhibitor (i.e., an inhibitor of any of the components of androgen signaling pathways). In some specific embodiments, the second agent is an inhibitor of androgen synthesis. In some specific embodiments, the second agent binds to androgen receptors. In some specific embodiments, the second agent is a luteinizing hormone-releasing hormone (LHRH) agonists. In some specific embodiments, the second agent is a gonadotropin-releasing hormone (GnRH) antagonist. In other embodiments, the second agent is an agent used in immunotherapy for prostate cancer. In some specific embodiments, the second agent is an immunocheckpoint inhibitor. In other embodiments, the second agent is a radiopharmaceutical.

In some specific embodiments, the second agent is docetaxel. In other specific embodiments, the second agent is mitoxantrone. In other specific embodiments, the second agent is cabazitaxel (Jevtana®). In other specific embodiments, the second agent is niraparib. In other specific embodiments, the second agent is olaparib (Lynparza®). In other specific embodiments, the second agent is rucaparib (Rubraca®). In other specific embodiments, the second agent is abiraterone acetate (Zytiga®/Yonsa®). In other specific embodiments, the second agent is Ketoconazole (Nizoral®). In other specific embodiments, the second agent is an inhibitor of CYP17 enzyme family. In other specific embodiments, the second agent is bicalutamide (Casodex®). In other specific embodiments, the second agent is flutamide. In other specific embodiments, the second agent is nilutamide (Nilandron®). In other specific embodiments, the second agent is apalutamide (Erleada®). In other specific embodiments, the second agent is darolutamide (Nubega®). In other specific embodiments, the second agent is enzalutamide (Xtandi®). In other specific embodiments, the second agent is leuprolide acetate (ELIGARD®/Lupron Depot®). In other specific embodiments, the second agent is goserelin (Zoladex®). In other specific embodiments, the second agent is degarelix (Firmagon®). In other specific embodiments, the second agent is sipuleucel-T (Provenge®). In other specific embodiments, the second agent is pembrolizumab. In other specific embodiments, the second agent is ADXS-PSA. In other specific embodiments, the second agent is radium-223 (Xofigo®).

In some specific embodiments, the second agent is docetaxel with a steroid such as prednisone or methylprednisolone. In other specific embodiments, the second agent is mitoxantrone with a steroid such as prednisone or methylprednisolone. In other specific embodiments, the second agent is cabazitaxel (Jevtana®) with a steroid such as prednisone or methylprednisolone. In other specific embodiments, the second agent is niraparib with a steroid such as prednisone or methylprednisolone. In other specific embodiments, the second agent is olaparib (Lynparza®) with a steroid such as prednisone or methylprednisolone. In other specific embodiments, the second agent is rucaparib (Rubraca®) with a steroid such as prednisone or methylprednisolone. In other specific embodiments, the second agent is abiraterone acetate (Zytiga®/Yonsa®) with a steroid such as prednisone or methylprednisolone. In other specific embodiments, the second agent is Ketoconazole (Nizoral®) with a steroid such as prednisone or methylprednisolone. In other specific embodiments, the second agent is an inhibitor of CYP17 enzyme family with a steroid such as prednisone or methylprednisolone. In other specific embodiments, the second agent is bicalutamide (Casodex®) with a steroid such as prednisone or methylprednisolone. In other specific embodiments, the second agent is flutamide with a steroid such as prednisone or methylprednisolone. In other specific embodiments, the second agent is nilutamide (Nilandron®) with a steroid such as prednisone or methylprednisolone. In other specific embodiments, the second agent is apalutamide (Erleada®) with a steroid such as prednisone or methylprednisolone. In other specific embodiments, the second agent is darolutamide (Nubega®) with a steroid such as prednisone or methylprednisolone. In other specific embodiments, the second agent is enzalutamide (Xtandi®) with a steroid such as prednisone or methylprednisolone. In other specific embodiments, the second agent is leuprolide acetate (ELIGARD®/Lupron Depot®) with a steroid such as prednisone or methylprednisolone. In other specific embodiments, the second agent is Goserelin (Zoladex®) with a steroid such as prednisone or methylprednisolone. In other specific embodiments, the second agent is degarelix (Firmagon®) with a steroid such as prednisone or methylprednisolone. In other specific embodiments, the second agent is sipuleucel-T (Provenge®) with a steroid such as prednisone or methylprednisolone. In other specific embodiments, the second agent is pembrolizumab with a steroid such as prednisone or methylprednisolone. In other specific embodiments, the second agent is ADXS-PSA with a steroid such as prednisone or methylprednisolone. In other specific embodiments, the second agent is radium-223 (Xofigo®) with a steroid such as prednisone or methylprednisolone.

In some embodiments, the second agent described in this section is administered intravenously. In other embodiments, the second agent described in this section is administered subcutaneously. In other embodiments, the second agent described in this section is administered orally. In other embodiments, the second agent described in this section is administered intradermally. In other embodiments, the second agent described in this section is administered intramuscularly. In other embodiments, the second agent described in this section is administered intraperitoneally. In other embodiments, the second agent described in this section is administered topically. In other embodiments, the second agent described in this section is administered percutaneously. In other embodiments, the second agent described in this section is administered intranasally. In other embodiments, the second agent described in this section is administered intratumorally.

In some embodiments, the first pharmaceutical composition and the second agent are co-administered simultaneously. In other embodiments, the first pharmaceutical composition is administered prior to administration of the second agent. In specific embodiments, the first pharmaceutical composition is administered about 1 hour prior to administration of the second agent. In specific embodiments, the first pharmaceutical composition is administered about 2 hours prior to administration of the second agent. In specific embodiments, the first pharmaceutical composition is administered about 3 hours prior to administration of the second agent. In specific embodiments, the first pharmaceutical composition is administered about 4 hours prior to administration of the second agent. In specific embodiments, the first pharmaceutical composition is administered about 5 hours prior to administration of the second agent. In specific embodiments, the first pharmaceutical composition is administered about 6 hours prior to administration of the second agent. In specific embodiments, the first pharmaceutical composition is administered about 7 hours prior to administration of the second agent. In specific embodiments, the first pharmaceutical composition is administered about 8 hours prior to administration of the second agent. In specific embodiments, the first pharmaceutical composition is administered about 9 hours prior to administration of the second agent. In specific embodiments, the first pharmaceutical composition is administered about 10 hours prior to administration of the second agent. In specific embodiments, the first pharmaceutical composition is administered about 11 hours prior to administration of the second agent. In specific embodiments, the first pharmaceutical composition is administered about 12 hours prior to administration of the second agent. In specific embodiments, the first pharmaceutical composition is administered about 1 day prior to administration of the second agent. In specific embodiments, the first pharmaceutical composition is administered about 2 days prior to administration of the second agent. In specific embodiments, the first pharmaceutical composition is administered about 3 days prior to administration of the second agent. In specific embodiments, the first pharmaceutical composition is administered about 4 days prior to administration of the second agent. In specific embodiments, the first pharmaceutical composition is administered about 5 days prior to administration of the second agent. In specific embodiments, the first pharmaceutical composition is administered about 6 days prior to administration of the second agent. In specific embodiments, the first pharmaceutical composition is administered about 1 week prior to administration of the second agent. In specific embodiments, the first pharmaceutical composition is administered about 2 weeks prior to administration of the second agent. In specific embodiments, the first pharmaceutical composition is administered about 3 weeks prior to administration of the second agent. In specific embodiments, the first pharmaceutical composition is administered about 4 weeks prior to administration of the second agent. In specific embodiments, the first pharmaceutical composition is administered about 5 weeks prior to administration of the second agent. In specific embodiments, the first pharmaceutical composition is administered about 6 weeks prior to administration of the second agent. In specific embodiments, the first pharmaceutical composition is administered about 7 weeks prior to administration of the second agent. In specific embodiments, the first pharmaceutical composition is administered about 8 weeks prior to administration of the second agent. In specific embodiments, the first pharmaceutical composition is administered about 9 weeks prior to administration of the second agent. In specific embodiments, the first pharmaceutical composition is administered about 10 weeks prior to administration of the second agent. In specific embodiments, the first pharmaceutical composition is administered about 11 weeks prior to administration of the second agent. In specific embodiments, the first pharmaceutical composition is administered about 12 weeks prior to administration of the second agent. In specific embodiments, the first pharmaceutical composition is administered about 1 month prior to administration of the second agent. In specific embodiments, the first pharmaceutical composition is administered about 2 months prior to administration of the second agent. In specific embodiments, the first pharmaceutical composition is administered about 3 months prior to administration of the second agent. In specific embodiments, the first pharmaceutical composition is administered about 4 months prior to administration of the second agent. In specific embodiments, the first pharmaceutical composition is administered about 5 months prior to administration of the second agent. In specific embodiments, the first pharmaceutical composition is administered about 6 months prior to administration of the second agent. In specific embodiments, the first pharmaceutical composition is administered more than 6 months prior to administration of the second agent.

In some embodiments, the first pharmaceutical composition is administered after administration of the second agent. In specific embodiments, the first pharmaceutical composition is administered about 1 hour after administration of the second agent. In specific embodiments, the first pharmaceutical composition is administered about 2 hours after administration of the second agent. In specific embodiments, the first pharmaceutical composition is administered about 3 hours after administration of the second agent. In specific embodiments, the first pharmaceutical composition is administered about 4 hours after administration of the second agent. In specific embodiments, the first pharmaceutical composition is administered about 5 hours after administration of the second agent. In specific embodiments, the first pharmaceutical composition is administered about 6 hours after administration of the second agent. In specific embodiments, the first pharmaceutical composition is administered about 7 hours after administration of the second agent. In specific embodiments, the first pharmaceutical composition is administered about 8 hours after administration of the second agent. In specific embodiments, the first pharmaceutical composition is administered about 9 hours after administration of the second agent. In specific embodiments, the first pharmaceutical composition is administered about 10 hours after administration of the second agent. In specific embodiments, the first pharmaceutical composition is administered about 11 hours after administration of the second agent. In specific embodiments, the first pharmaceutical composition is administered about 12 hours after administration of the second agent. In specific embodiments, the first pharmaceutical composition is administered about 1 day after administration of the second agent. In specific embodiments, the first pharmaceutical composition is administered about 2 days after administration of the second agent. In specific embodiments, the first pharmaceutical composition is administered about 3 days after administration of the second agent. In specific embodiments, the first pharmaceutical composition is administered about 4 days after administration of the second agent. In specific embodiments, the first pharmaceutical composition is administered about 5 days after administration of the second agent. In specific embodiments, the first pharmaceutical composition is administered about 6 days after administration of the second agent. In specific embodiments, the first pharmaceutical composition is administered about 1 week after administration of the second agent. In specific embodiments, the first pharmaceutical composition is administered about 2 weeks after administration of the second agent. In specific embodiments, the first pharmaceutical composition is administered about 3 weeks after administration of the second agent. In specific embodiments, the first pharmaceutical composition is administered about 4 weeks after administration of the second agent. In specific embodiments, the first pharmaceutical composition is administered about 5 weeks after administration of the second agent. In specific embodiments, the first pharmaceutical composition is administered about 6 weeks after administration of the second agent. In specific embodiments, the first pharmaceutical composition is administered about 7 weeks after administration of the second agent. In specific embodiments, the first pharmaceutical composition is administered about 8 weeks after administration of the second agent. In specific embodiments, the first pharmaceutical composition is administered about 9 weeks after administration of the second agent. In specific embodiments, the first pharmaceutical composition is administered about 10 weeks after administration of the second agent. In specific embodiments, the first pharmaceutical composition is administered about 11 weeks after administration of the second agent. In specific embodiments, the first pharmaceutical composition is administered about 12 weeks after administration of the second agent. In specific embodiments, the first pharmaceutical composition is administered about 1 month after administration of the second agent. In specific embodiments, the first pharmaceutical composition is administered about 2 months after administration of the second agent. In specific embodiments, the first pharmaceutical composition is administered about 3 months after administration of the second agent. In specific embodiments, the first pharmaceutical composition is administered about 4 months after administration of the second agent. In specific embodiments, the first pharmaceutical composition is administered about 5 months after administration of the second agent. In specific embodiments, the first pharmaceutical composition is administered about 6 months after administration of the second agent. In specific embodiments, the first pharmaceutical composition is administered more than 6 months after administration of the second agent.

In some embodiments, the second pharmaceutical composition and the second agent are co-administered simultaneously. In other embodiments, the second pharmaceutical composition is administered prior to administration of the second agent. In specific embodiments, the second pharmaceutical composition is administered about 1 hour prior to administration of the second agent. In specific embodiments, the second pharmaceutical composition is administered about 2 hours prior to administration of the second agent. In specific embodiments, the second pharmaceutical composition is administered about 3 hours prior to administration of the second agent. In specific embodiments, the second pharmaceutical composition is administered about 4 hours prior to administration of the second agent. In specific embodiments, the second pharmaceutical composition is administered about 5 hours prior to administration of the second agent. In specific embodiments, the second pharmaceutical composition is administered about 6 hours prior to administration of the second agent. In specific embodiments, the second pharmaceutical composition is administered about 7 hours prior to administration of the second agent. In specific embodiments, the second pharmaceutical composition is administered about 8 hours prior to administration of the second agent. In specific embodiments, the second pharmaceutical composition is administered about 9 hours prior to administration of the second agent. In specific embodiments, the second pharmaceutical composition is administered about 10 hours prior to administration of the second agent. In specific embodiments, the second pharmaceutical composition is administered about 11 hours prior to administration of the second agent. In specific embodiments, the second pharmaceutical composition is administered about 12 hours prior to administration of the second agent. In specific embodiments, the second pharmaceutical composition is administered about 1 day prior to administration of the second agent. In specific embodiments, the second pharmaceutical composition is administered about 2 days prior to administration of the second agent. In specific embodiments, the second pharmaceutical composition is administered about 3 days prior to administration of the second agent. In specific embodiments, the second pharmaceutical composition is administered about 4 days prior to administration of the second agent. In specific embodiments, the second pharmaceutical composition is administered about 5 days prior to administration of the second agent. In specific embodiments, the second pharmaceutical composition is administered about 6 days prior to administration of the second agent. In specific embodiments, the second pharmaceutical composition is administered about 1 week prior to administration of the second agent. In specific embodiments, the second pharmaceutical composition is administered about 2 weeks prior to administration of the second agent. In specific embodiments, the second pharmaceutical composition is administered about 3 weeks prior to administration of the second agent. In specific embodiments, the second pharmaceutical composition is administered about 4 weeks prior to administration of the second agent. In specific embodiments, the second pharmaceutical composition is administered about 5 weeks prior to administration of the second agent. In specific embodiments, the second pharmaceutical composition is administered about 6 weeks prior to administration of the second agent. In specific embodiments, the second pharmaceutical composition is administered about 7 weeks prior to administration of the second agent. In specific embodiments, the second pharmaceutical composition is administered about 8 weeks prior to administration of the second agent. In specific embodiments, the second pharmaceutical composition is administered about 9 weeks prior to administration of the second agent. In specific embodiments, the second pharmaceutical composition is administered about 10 weeks prior to administration of the second agent. In specific embodiments, the second pharmaceutical composition is administered about 11 weeks prior to administration of the second agent. In specific embodiments, the second pharmaceutical composition is administered about 12 weeks prior to administration of the second agent. In specific embodiments, the second pharmaceutical composition is administered about 1 month prior to administration of the second agent. In specific embodiments, the second pharmaceutical composition is administered about 2 months prior to administration of the second agent. In specific embodiments, the second pharmaceutical composition is administered about 3 months prior to administration of the second agent. In specific embodiments, the second pharmaceutical composition is administered about 4 months prior to administration of the second agent. In specific embodiments, the second pharmaceutical composition is administered about 5 months prior to administration of the second agent. In specific embodiments, the second pharmaceutical composition is administered about 6 months prior to administration of the second agent. In specific embodiments, the second pharmaceutical composition is administered more than 6 months prior to administration of the second agent.

In some embodiments, the second pharmaceutical composition is administered after administration of the second agent. In specific embodiments, the second pharmaceutical composition is administered about 1 hour after administration of the second agent. In specific embodiments, the second pharmaceutical composition is administered about 2 hours after administration of the second agent. In specific embodiments, the second pharmaceutical composition is administered about 3 hours after administration of the second agent. In specific embodiments, the second pharmaceutical composition is administered about 4 hours after administration of the second agent. In specific embodiments, the second pharmaceutical composition is administered about 5 hours after administration of the second agent. In specific embodiments, the second pharmaceutical composition is administered about 6 hours after administration of the second agent. In specific embodiments, the second pharmaceutical composition is administered about 7 hours after administration of the second agent. In specific embodiments, the second pharmaceutical composition is administered about 8 hours after administration of the second agent. In specific embodiments, the second pharmaceutical composition is administered about 9 hours after administration of the second agent. In specific embodiments, the second pharmaceutical composition is administered about 10 hours after administration of the second agent. In specific embodiments, the second pharmaceutical composition is administered about 11 hours after administration of the second agent. In specific embodiments, the second pharmaceutical composition is administered about 12 hours after administration of the second agent. In specific embodiments, the second pharmaceutical composition is administered about 1 day after administration of the second agent. In specific embodiments, the second pharmaceutical composition is administered about 2 days after administration of the second agent. In specific embodiments, the second pharmaceutical composition is administered about 3 days after administration of the second agent. In specific embodiments, the second pharmaceutical composition is administered about 4 days after administration of the second agent. In specific embodiments, the second pharmaceutical composition is administered about 5 days after administration of the second agent. In specific embodiments, the second pharmaceutical composition is administered about 6 days after administration of the second agent. In specific embodiments, the second pharmaceutical composition is administered about 1 week after administration of the second agent. In specific embodiments, the second pharmaceutical composition is administered about 2 weeks after administration of the second agent. In specific embodiments, the second pharmaceutical composition is administered about 3 weeks after administration of the second agent. In specific embodiments, the second pharmaceutical composition is administered about 4 weeks after administration of the second agent. In specific embodiments, the second pharmaceutical composition is administered about 5 weeks after administration of the second agent. In specific embodiments, the second pharmaceutical composition is administered about 6 weeks after administration of the second agent. In specific embodiments, the second pharmaceutical composition is administered about 7 weeks after administration of the second agent. In specific embodiments, the second pharmaceutical composition is administered about 8 weeks after administration of the second agent. In specific embodiments, the second pharmaceutical composition is administered about 9 weeks after administration of the second agent. In specific embodiments, the second pharmaceutical composition is administered about 10 weeks after administration of the second agent. In specific embodiments, the second pharmaceutical composition is administered about 11 weeks after administration of the second agent. In specific embodiments, the second pharmaceutical composition is administered about 12 weeks after administration of the second agent. In specific embodiments, the second pharmaceutical composition is administered about 1 month after administration of the second agent. In specific embodiments, the second pharmaceutical composition is administered about 2 months after administration of the second agent. In specific embodiments, the second pharmaceutical composition is administered about 3 months after administration of the second agent. In specific embodiments, the second pharmaceutical composition is administered about 4 months after administration of the second agent. In specific embodiments, the second pharmaceutical composition is administered about 5 months after administration of the second agent. In specific embodiments, the second pharmaceutical composition is administered about 6 months after administration of the second agent. In specific embodiments, the second pharmaceutical composition is administered more than 6 months after administration of the second agent.

### 5.6.5 Patient Populations

In some embodiments, the methods provided herein are to administer to a subject who is suffering from prostate cancer. In other embodiments, the methods provided herein are to administer to a subject who is susceptible to prostate cancer. In other embodiments, the methods provided herein are to administer to a subject who is at risk of prostate cancer.

As is well known in the field, a widely used staging system to evaluate the advancement of prostate cancer is the American Joint Committee on Cancer (AJCC) TNM system. The TNM system for prostate cancer is based on 5 aspects of information: (i) the extent of the primary tumor (T category), which can be further divided in two sub-categories: the clinical T category (cT) based on physical exam (including a digital rectal exam), prostate biopsy, and any imaging tests; and pathologic T category (pT) based on the surgically removed prostate; (ii) whether the cancer has spread to nearby lymph nodes (N category); (iii) whether the cancer has spread (metastasized) to other parts of the body (M category); (iv) PSA level; and (v) the Gleason score of the prostate biopsy. In some embodiments, the methods provided herein are to administer to a subject who is diagnosed as cT1, N0, M0, Grade Group 1 (Gleason score 6 or less), and PSA less than 10. In other embodiments, the methods provided herein are to administer to a subject who is diagnosed as cT2a, N0, M0, Grade Group 1 (Gleason score 6 or less), and PSA less than 10. In other embodiments, the methods provided herein are to administer to a subject who is diagnosed as pT2, N0, M0, Grade Group 1 (Gleason score 6 or less), and PSA less than 10. In other embodiments, the methods provided herein are to administer to a subject who is diagnosed as cT1, N0, M0, Grade Group 1 (Gleason score 6 or less), and PSA at least 10 but less than 20. In other embodiments, the methods provided herein are to administer to a subject who is diagnosed as cT2a or pT2, N0, M0, Grade Group 1 (Gleason score 6 or less), and PSA at least 10 but less than 20. In other embodiments, the methods provided herein are to administer to a subject who is diagnosed as cT2b or cT2c, N0, M0, Grade Group 1 (Gleason score 6 or less), and PSA less than 20. In other embodiments, the methods provided herein are to administer to a subject who is diagnosed as T1 or T2, N0, M0, Grade Group 2 (Gleason score 7), and PSA less than 20. In other embodiments, the methods provided herein are to administer to a subject who is diagnosed as T1 or T2, N0, M0, Grade Group 2 (Gleason score 7 or 8), and PSA less than 20. In other embodiments, the methods provided herein are to administer to a subject who is diagnosed as T1 or T2, N0, M0, Grade Group 1 to 4 (Gleason score 8 or less), and PSA less than 20. In other embodiments, the methods provided herein are to administer to a subject who is diagnosed as T3 or T4, N0, M0, Grade Group 1 to 4 (Gleason score 8 or less), and any PSA. In other embodiments, the methods provided herein are to administer to a subject who is diagnosed as any T, N0, M0, Grade Group 5 (Gleason score 9 or 10), and any PSA. In other embodiments, the methods provided herein are to administer to a subject who is diagnosed as any T, NI, M0, any Grade Group for Gleason score, and any PSA. In other embodiments, the methods provided herein are to administer to a subject who is diagnosed as any T, any N, M1, any Grade Group for Gleason score, and any PSA.

### 5.6.6 A Kit

Also provided herein are kits that can be used to perform the methods described in this section (i.e., Section 5.6). Thus, in certain embodiments, the kit provided herein includes one or more containers and instructions for use, wherein the one or more containers comprise a composition (e.g., pharmaceutical, immunogenic or vaccine composition) provided herein. In some certain embodiments, a kit provided herein includes containers that each contains the active ingredients in a pharmaceutical composition suitable for intravenous administration for performing the methods described herein. In some specific embodiments, a kit provided herein includes two or more containers and instructions for use, wherein one of the containers comprises an arenavirus particle described in Section 5.3 and another container that comprises a second agent described in Section 5.6.4.

Also provided herein are kits that include one or more containers and instructions for use, wherein the one or more containers comprise a composition (e.g., pharmaceutical, immunogenic or vaccine composition) in a pharmaceutical composition suitable for intravenous administration provided herein, and an apparatus suitable for performing intravenous administration, such as rigid or semi-rigid open container, plastic or closed container, tubing, drip chamber, and other accessories to the tubing that are needed to move the fluid from the container to the patient's vein, and needles. In some specific embodiments, a kit provided herein includes two or more containers and instructions for use, wherein one of the containers comprises an arenavirus particle described in Section 5.3 and another container that comprises a second agent described in Section 5.6.4, and an apparatus suitable for performing intravenous administration, such as rigid or semi-rigid open container, plastic or closed container, tubing, drip chamber, and other accessories to the tubing that are needed to move the fluid from the container to the patient's vein, and needles.

### 5.7 Assay

### 5.7.1 Arenavirus Detection Assays

Provided herein are techniques known in the art that could detect the existence of an arenavirus S segment as described in Section 5.1 or tri-segmented arenavirus particle as described in Section 5.3 in various samples, such as saliva, feces, blood, and urine of treated animals or treated patients, as exemplified in Table 10. Summary of Sample Collection for Central Laboratory Analyses. For example, RT-PCR can be used with primers that are specific to an arenavirus to detect and quantify an arenavirus S segment that has been engineered to carry a heterologous ORF encoding a prostate cancer-related antigen as described herein or a tri-segmented arenavirus particle as described herein. Western blot, ELISA, radioimmunoassay, immunoprecipitation, immunocytochemistry, or immunocytochemistry in conjunction with FACS can be used to quantify the gene products of the arenavirus S segment or tri-segmented arenavirus particle.

### 5.7.2 Assay to Measure Infectivity

Any assay well known in the art can be used for measuring the infectivity of an arenavirus particle preparation. For example, determination of the virus/vector titer can be done by a “focus forming unit assay” (FFU assay). In brief, complementing cells, e.g., HEK293-TVL cells are plated and inoculated with different dilutions of a virus/vector sample. After an incubation period, to allow cells to form a monolayer and virus to attach to cells, the monolayer is covered with Methylcellulose. When the plates are further incubated, the original infected cells release viral progeny. Due to the Methylcellulose overlay the spread of the new viruses is restricted to neighboring cells. Consequently, each infectious particle produces a circular zone of infected cells called a Focus. Such Foci can be made visible and by that countable using antibodies against LCMV-NP or another protein expressed by the arenavirus particle or the tri-segmented arenavirus particle and an HRP-based color reaction. The titer of a virus/vector can be calculated in focus-forming units per milliliter (FFU/mL). In a similar way, the proportion of tri-segmented, replication competent virus particles can be determined. Instead of complementing cells, non-complementing cell lines are used, e.g. HEK293. This allows only trisegmented virus particles to infect neighboring cells. The titer of the replication competent virus/vector (RCV) can be calculated in focus-forming units per milliliter (RCV FFU/mL) Similarly, the infectivity can be measured in clinical setting in samples from treated patients, as exemplified in Table 10. Summary of Sample Collection for Central Laboratory Analyses.

### 5.7.3 Growth of an Arenavirus Particle

Growth of an arenavirus particle described herein can be assessed by any method known in the art or described herein. Viral growth may be determined by inoculating a defined amount/concentration of arenavirus particles described herein into cell cultures (e.g., Vero cells or BHK-21 cells). After incubation of the virus for a specified time, the virus containing supernatant is collected using standard methods and the infectivity can be measured using herein described assays.

### 5.7.4 Serum ELISA

Determination of the humoral immune response upon vaccination of animals (e.g., mice, guinea pigs) can be done by antigen-specific serum ELISA's (enzyme-linked immunosorbent assays). In brief, plates are coated with antigen (e.g., recombinant protein), blocked to avoid unspecific binding of antibodies and incubated with serial dilutions of sera. After incubation, bound serum-antibodies can be detected, e.g., using an enzyme-coupled anti-species (e.g., mouse, guinea pig)-specific antibody (detecting total IgG or IgG subclasses) and subsequent color reaction. Antibody titers can be determined as, e.g., endpoint geometric mean titer.

### 5.7.5 Assay to Measure the Neutralizing Activity of Induced Antibodies

Determination of the neutralizing antibodies in sera is performed with the following cell assay using ARPE-19 cells from ATCC and a GFP-tagged virus. In addition supplemental guinea pig serum as a source of exogenous complement is used. The assay is started with seeding of 6.5×10³ cells/well (50 μl/well) in a 384 well plate one or two days before using for neutralization. The neutralization is done in 96-well sterile tissue culture plates without cells for 1 h at 37° C. After the neutralization incubation step the mixture is added to the cells and incubated for additional 4 days for GFP-detection with a plate reader. A positive neutralizing human sera is used as assay positive control on each plate to check the reliability of all results. Titers (EC50) are determined using a 4 parameter logistic curve fitting. As additional testing the wells are checked with a fluorescence microscope. Similarly, neutralizing activity of induced antibodies can be measured in clinical setting, as exemplified in Table 10. Summary of Sample Collection for Central Laboratory Analyses.

### 5.7.6 Plaque Reduction Assay

In brief, plaque reduction (neutralization) assays for LCMV can be performed by use of a replication-competent or -deficient LCMV that is encoding a reporter gene (e.g. green fluorescent protein (GFP), 5% rabbit serum may be used as a source of exogenous complement, and plaques can be enumerated by fluorescence microscopy. Neutralization titers may be defined as the highest dilution of serum that results in a 50%, 75%, 90% or 95% reduction in plaques, compared with that in control (pre-immune) serum samples.

qPCR LCMV RNA genomes are isolated using QIAamp Viral RNA mini Kit (QIAGEN), according to the protocol provided by the manufacturer. LCMV RNA genome equivalents are detected by quantitative PCR carried out on an StepOnePlus Real Time PCR System (Applied Biosystems) with SuperScript® III Platinum® One-Step qRT-PCR Kit (Invitrogen) and primers and probes (FAM reporter and NFQ-MGB Quencher) specific for part of the LCMV NP coding region or another genomic stretch of the arenavirus particle or the tri-segmented arenavirus particle. The temperature profile of the reaction may be: 30 min at 60° C., 2 min at 95° C., followed by 45 cycles of 15 s at 95° C., 30 s at 56° C. RNA can be quantified by comparison of the sample results to a standard curve prepared from a log 10 dilution series of a spectrophotometrically quantified, in vitro-transcribed RNA fragment, corresponding to a fragment of the LCMV NP coding sequence or another genomic stretch of the arenavirus particle or the tri-segmented arenavirus particle containing the primer and probe binding sites.

### 5.7.7 Western Blotting

Infected cells grown in tissue culture flasks or in suspension are lysed at indicated time points post infection using RIPA buffer (Thermo Scientific) or used directly without cell-lysis. Samples are heated to 99° C. for 10 minutes with reducing agent and NuPage LDS Sample buffer (NOVEX) and chilled to room temperature before loading on 4-12% SDS-gels for electrophoresis. Proteins are blotted onto membranes using Invitrogens iBlot Gel transfer Device and visualized by Ponceau staining. Finally, the preparations are probed with primary antibodies directed against proteins of interest and alkaline phosphatase conjugated secondary antibodies followed by staining with 1-Step NBT/BCIP solution (INVITROGEN).

### 5.7.8 MHC-Peptide Multimer Staining Assay for Detection of Antigen-Specific CD8+ T-Cells

Any assay well known in the art can be used to test antigen-specific CD8+ T-cell responses. For example, the MHC-peptide tetramer staining assay can be used (see, e.g., Altman J. D. et al., Science. 1996; 274:94-96; and Murali-Krishna K. et al., Immunity. 1998; 8:177-187). Briefly, the assay comprises the following steps, a tetramer assay is used to detect the presence of antigen specific T-cells. In order to detect an antigen-specific T-cell, it must bind to both, the peptide and the tetramer of MHC molecules custom made for a defined antigen specificity and MHC haplotype of T-cells (typically fluorescently labeled). The tetramer is then detected by flow cytometry via the fluorescent label.

### 5.7.9 ELISPOT Assay for Detection of Antigen-Specific T-Cells

Any assay well known in the art can be used to test antigen-specific T-cell responses. For example, the ELISPOT assay can be used (see, e.g., Czerkinsky C. C. et al., J Immunol Methods. 1983; 65:109-121; and Hutchings P. R. et al., J Immunol Methods. 1989; 120:1-8). As exemplified in Table 10. Summary of Sample Collection for Central Laboratory Analyses, cytokines such as but not limited to IFN-γ can be measured by the ELISPOT assay. Briefly, the assay comprises the following steps: An immunospot plate is coated with an anti-cytokine antibody. Cells are incubated in the immunospot plate with peptides derived from the antigen of interest. Antigen-specific cells secrete cytokines, which bind to the coated antibodies. The cells are then washed off and a second biotyinlated-anticytokine antibody is added to the plate and visualized with an avidin-HRP system or other appropriate methods.

### 5.7.10 Intracellular Cytokine Assay for Detection of Functionality of CD8+ and CD4+ T-Cells.

Any assay well known in the art can be used to test the functionality of CD8+ and CD4+ T cell responses. For example, the intracellular cytokine assay combined with flow cytometry can be used as exemplified but not limited to Table 10. Summary of Sample Collection for Central Laboratory Analyses (see, e.g., Suni M. A. et al., J Immunol Methods. 1998; 212:89-98; Nomura L. E. et al., Cytometry. 2000; 40:60-68; and Ghanekar S. A. et al., Clinical and Diagnostic Laboratory Immunology. 2001; 8:628-63). Briefly, the assay comprises the following steps: upon activation of cells via specific peptides or protein, an inhibition of protein transport (e.g., brefeldin A) is added to retain the cytokines within the cell. After a defined period of incubation, typically 5 hours, a washing step follows, and antibodies to other cellular markers can be added to the cells. Cells are then fixed and permeablized. The flurochrome-conjugated anti-cytokine antibodies are added and the cells can be analyzed by flow cytometry.

### 5.7.11 Assay for Confirming Replication-Deficiency of Viral Vectors

Any assay well known in the art that determines concentration of infectious and replication-competent virus particles can also be used as a to measure replication-deficient viral particles in a sample. For example, FFU assays with non-complementing cells can be used for this purpose.

Furthermore, plaque-based assays are the standard method used to determine virus concentration in terms of plaque forming units (PFU) in a virus sample. Specifically, a confluent monolayer of non-complementing host cells is infected with the virus at varying dilutions and covered with a semi-solid medium, such as agar to prevent the virus infection from spreading indiscriminately. A viral plaque is formed when a virus successfully infects and replicates itself in a cell within the fixed cell monolayer, and spreads to surrounding cells (see, e.g., Kaufmann, S. H.; Kabelitz, D. (2002). Methods in Microbiology, Vol. 32: Immunology of Infection. Academic Press. ISBN 0-12-521532-0). Plaque formation can take 2-14 days, depending on the virus being analyzed. Plaques are generally counted manually and the results, in combination with the dilution factor used to prepare the plate, are used to calculate the number of plaque forming units per sample unit volume (PFU/mL). The PFU/mL result represents the number of infective replication-competent particles within the sample. When C-cells are used, the same assay can be used to titrate replication-deficient arenavirus particles or tri-segmented arenavirus particles.

### 5.7.12 Assay for Expression of Viral Antigen

Any assay well known in the art can be used for measuring expression of viral antigens. For example, FFU assays can be performed. For detection, mono- or polyclonal antibody preparation(s) against the respective viral antigens are used (transgene-specific FFU).

### 5.7.13 Animal Models

To investigate recombination and infectivity of an arenavirus particle described herein in vivo animal models can be used. In certain embodiments, the animal models that can be used to investigate recombination and infectivity of a tri-segmented arenavirus particle include mouse, guinea pig, rabbit, and monkeys. In a preferred embodiment, the animal models that can be used to investigate recombination and infectivity of an arenavirus include mouse. In a more specific embodiment, the mice can be used to investigate recombination and infectivity of an arenavirus particle are triple-deficient for type I interferon receptor, type II interferon receptor and recombination activating gene 1 (RAG1).

In certain embodiments, the animal models can be used to determine arenavirus infectivity and transgene stability. In some embodiments, viral RNA can be isolated from the serum of the animal model. Techniques are readily known by those skilled in the art. The viral RNA can be reverse transcribed and the cDNA carrying the arenavirus ORFs can be PCR-amplified with gene-specific primers. Flow cytometry can also be used to investigate arenavirus infectivity and transgene stability.

### 5.7.14 Assay for Prostate Cancer Progression

Any assay well known in the art can be used for assessing the progression of prostate cancer. As exemplified in Sections 6.3.6 and 6.3.6, prostate cancer progression and other relevant clinical parameters can be monitored. Briefly, the measurement of changing level of PSA, monitoring of the progression of target lesions and prostate, detection of bone metastases, as exemplified in Table 9. PCWG3 Criteria of Progression by Disease Manifestation, can be carried out with standard methods (see, e.g., Scher et al., 2016, J Clin Oncol, 34: 1402-18). Furthermore, parameters in hematology, clinical chemistry, urinalysis, coagulation, thyroid, serology, and prostate cancer-related testing can be monitored with standard clinical laboratory methods.

### 6. EXAMPLES

### 6.1 Vector Design and Transgene Stability During Serial Passaging

6.1.1 Vector Design and Transgene Stability of artLCMV-PAP-NP/PSA-GP

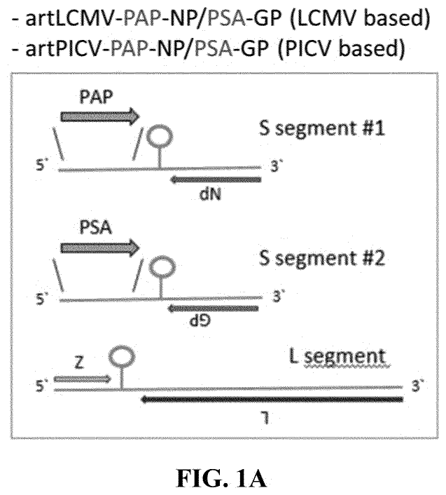

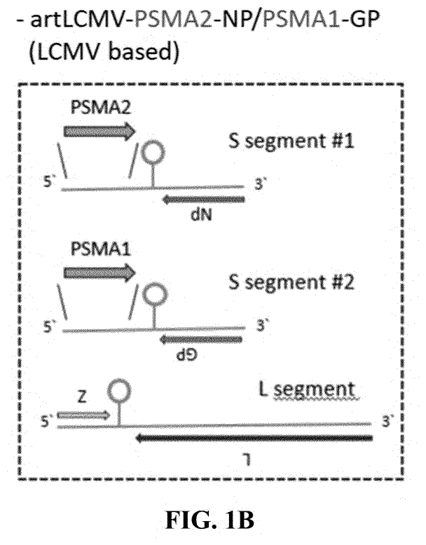

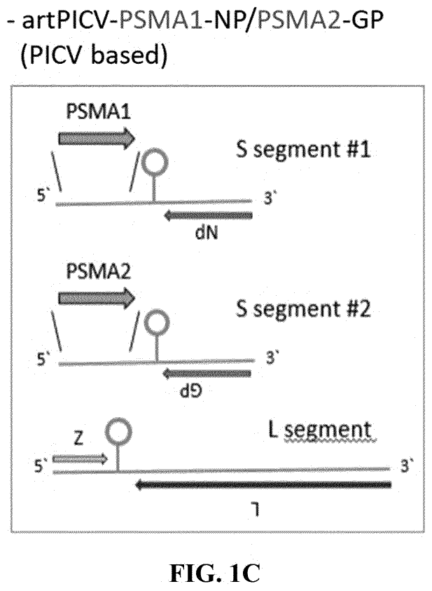

artLCMV-PAP-NP/PSA-GP is an attenuated, replication competent, tri-segmented vector based on LCMV clone 13 (LCMV c113) expressing the GP of LCMV strain WE instead of its endogenous glycoprotein (LCMV cl13/WE). As shown in FIG. 1A, the NP-S segment encodes for the human prostate cancer-related antigen PAP (SEQ ID NO. 5) and the GP-S segment encodes for PSA (SEQ ID NO. 6). The nucleotide sequences of both antigens were modified to be devoid of CpG dinucleotide motifs. The vector was generated de novo by electroporation of production cells using a five-plasmid co-transfection system, as described previously by Kallert et al. Nat Commun 2017; 8:15327.

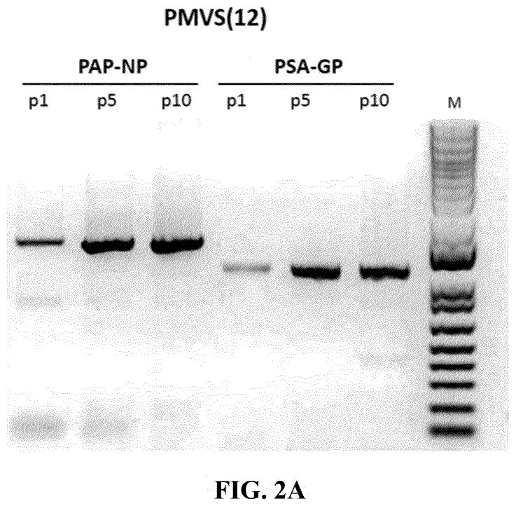

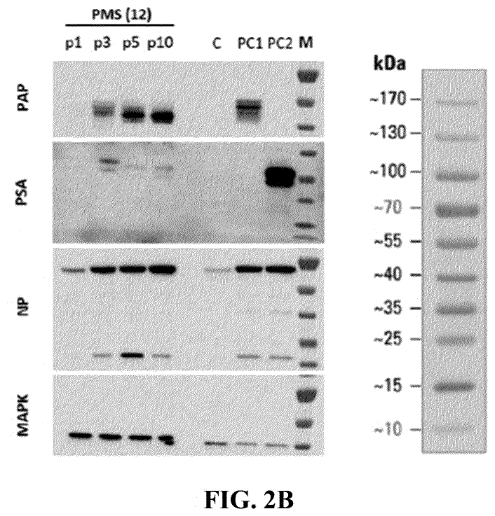

A PMVS of artLCMV-PAP-NP/PSA-GP vector, PMVS (12), was generated and genetic stability and expression of the encoded transgenes was analyzed by PCR and western blotting at increasing passage levels. As shown in FIG. 2A, the PAP and PSA transgenes were stable among all tested passage levels. As demonstrated in FIG. 2B, expression of PAP and PSA could be confirmed by western blotting.

6.1.2 Vector Design and Transgene Stability of artPICV-PAP-NP/PSA-GP

artPICV-PAP-NP/PSA-GP is an attenuated, replication competent, tri-segmented vector based on virulent strain passage 18 of Pichinde Virus (PIC; alternatively named PICV p18). As shown in FIG. 1A, the NP-S segment encodes for the human prostate cancer-related antigen PAP (SEQ ID NO. 5) and the GP-S segment encodes for PSA (SEQ ID NO. 6). The nucleotide sequences of both antigens were modified to be devoid of CpG dinucleotide motifs.

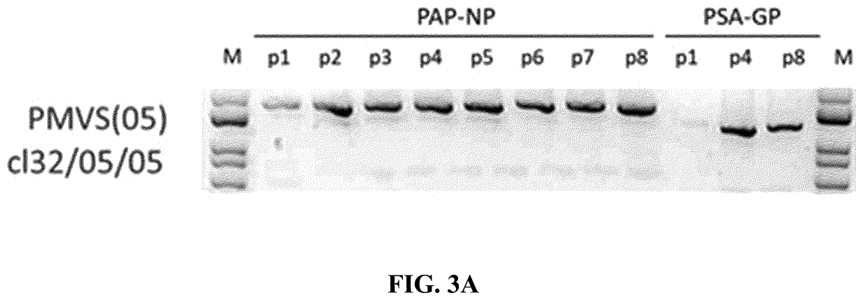

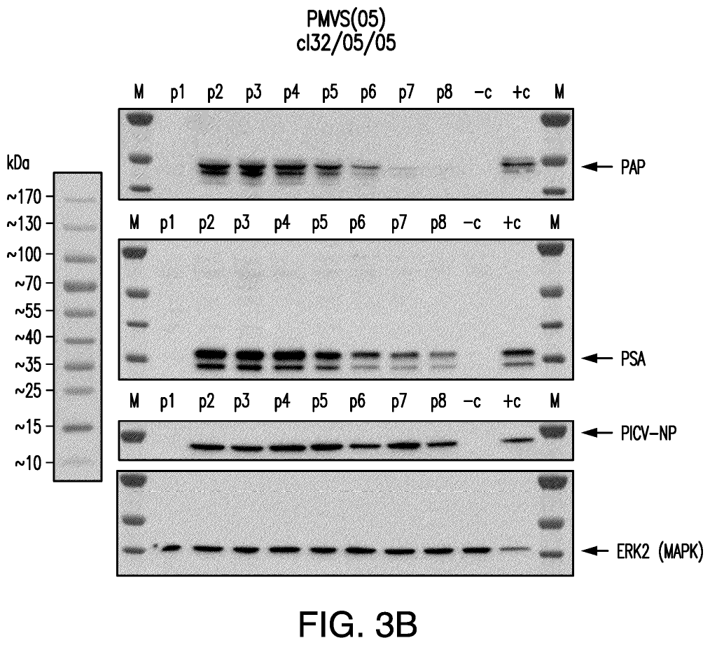

An artPICV-PAP-NP/PSA-GP PMVS candidate, PMVS (05) cl.32/05/05, was generated and genetic stability and expression of the encoded transgenes was analyzed by PCR and western blotting at increasing passage levels. As shown in FIG. 3A, the PAP and PSA transgenes were stable among all tested passage levels. As demonstrated in FIG. 3B, expression of PAP and PSA could be confirmed by western blotting.

6.1.3 Vector Design and Transgene Stability of artLCMV-PSMA2-NP/PSMA1-GP

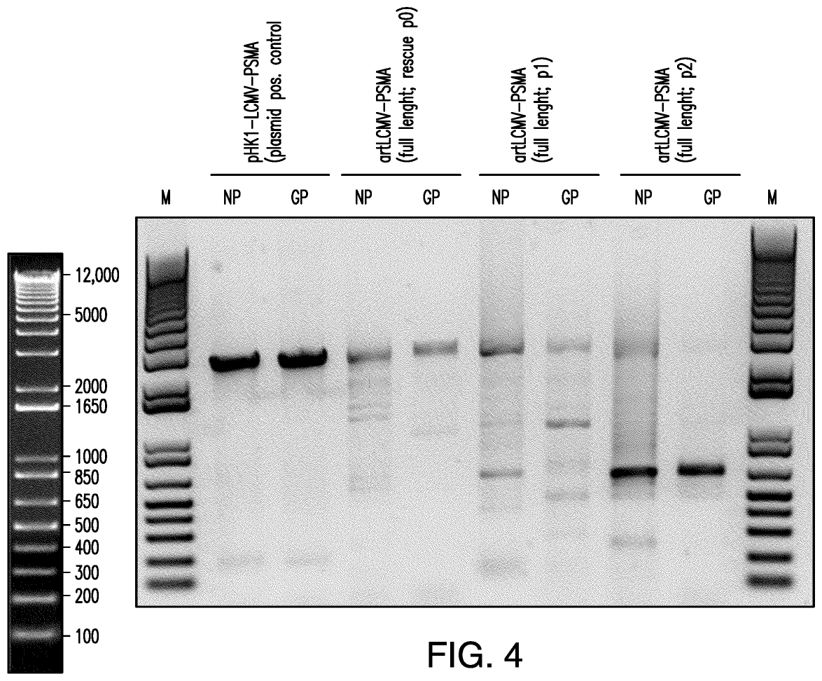

As shown in FIG. 4, an artLCMV vector encoding full length PSMA (artLCMV-PSMA) (SEQ ID NO. 9 consisting of 2253 bp, which is translated into 751 amino acids) exhibited major transgene instabilities during serial passaging. Therefore, the PSMA antigen was split into two parts, PSMA1 (SEQ ID NO. 3 and 343 amino acids or SEQ ID NO. 7 and 1032 bp), and PSMA2 (SEQ ID NO. 4 and 407 amino acids or SEQ ID NO. 8 and 1224 bp). Each part was encoded on one respective genomic S-Segment. Specifically, stop codon TGA was introduced for correct translation of PSMA1 transgene, and PSMA sequence was split before an ATG in order to keep a start codon.

artLCMV-PSMA2-NP/PSMA1-GP is an attenuated, replication competent, tri-segmented vector based on LCMV clone 13 (LCMV c113) expressing the GP of LCMV strain WE instead of its endogenous glycoprotein (LCMV c113/WE). The vector encodes the product of the human FOLH1 gene product, alternatively designated PSMA. The N-terminal amino acids 1-343 of PSMA and an artificially added stop codon were encoded by the ORF designated “PSMA1” (SEQ ID NO. 7) on the GP-S segment. The C-terminal amino acids 344-750 of PSMA, led by a pre-existing methionine on position 344 were encoded by the ORF designated “PSMA2” (SEQ ID NO. 8) on the NP-S segment. The nucleotide sequences were modified to be devoid of CpG dinucleotide motifs.

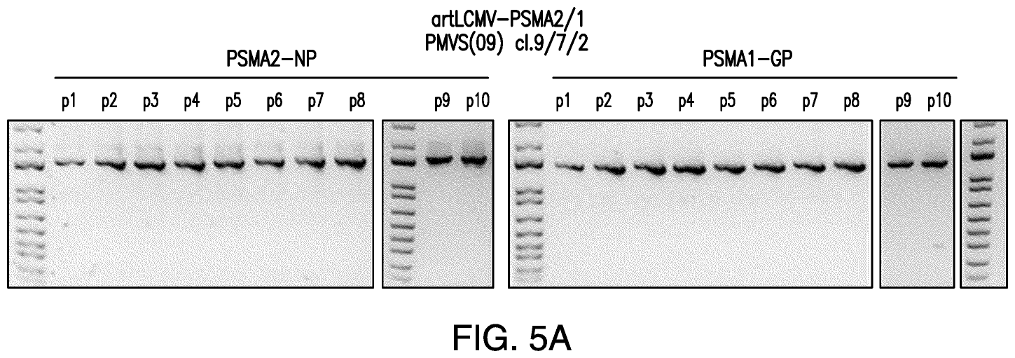

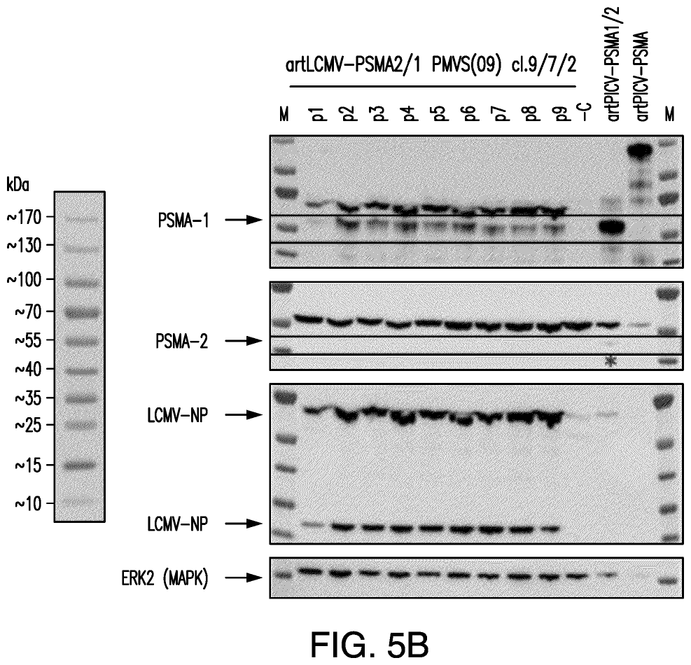

An artLCMV-PSMA2-NP/PSMA1-GP PMVS candidate, PMVS (09) cl.9/7/2, was generated and genetic stability and expression of the encoded transgenes was analyzed by PCR and western blotting at increasing passage levels. As shown in FIG. 5A, the PSMA1 and PSMA2 transgenes were stable among all tested passage levels. As demonstrated in FIG. 5B, expression of PSMA1 could be confirmed by western blotting. The protein expression results of PSMA2 were compromised by the poor quality of the antibody used, but sequencing of vector genome showed correct full length insert of the coding sequence.

6.1.4 Vector Design and Transgene Stability of artPICV-PSMA1-NP/PSMA2-GP

artPICV-PSMA1-NP/PSMA2-GP is an attenuated, replication competent, tri-segmented vector based on virulent strain passage 18 of Pichinde Virus (PICV; alternatively named PICV p18). As shown in FIG. 1C, the vector encoded the product of the human FOLH1 gene product, alternatively designated PSMA. The N-terminal amino acids 1-343 of PSMA and an artificially added stop codon were encoded by the ORF designated “PSMA1” (SEQ ID NO. 7) on the NP-S segment. The C-terminal amino acids 344-750 of PSMA, led by a pre-existing methionine on position 344 were encoded by the ORF designated “PSMA2” (SEQ ID NO. 8) on the GP-S segment. The nucleotide sequences were modified to be devoid of CpG dinucleotide motifs. As detailed below, artPICV-PSMA1-NP/PSMA2-GP was generated and found to stably encode and express the transgenes.

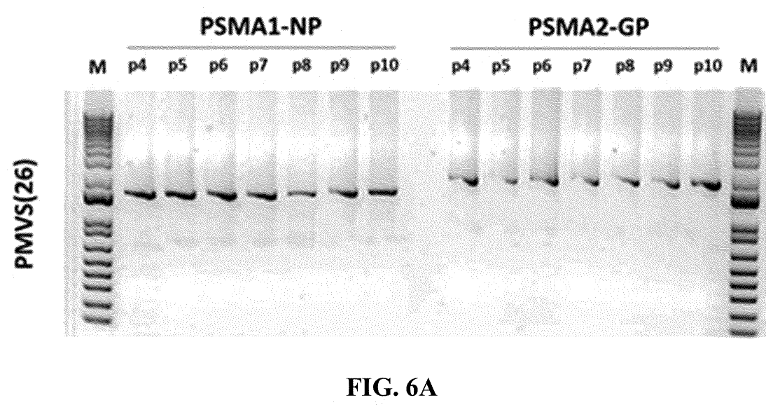

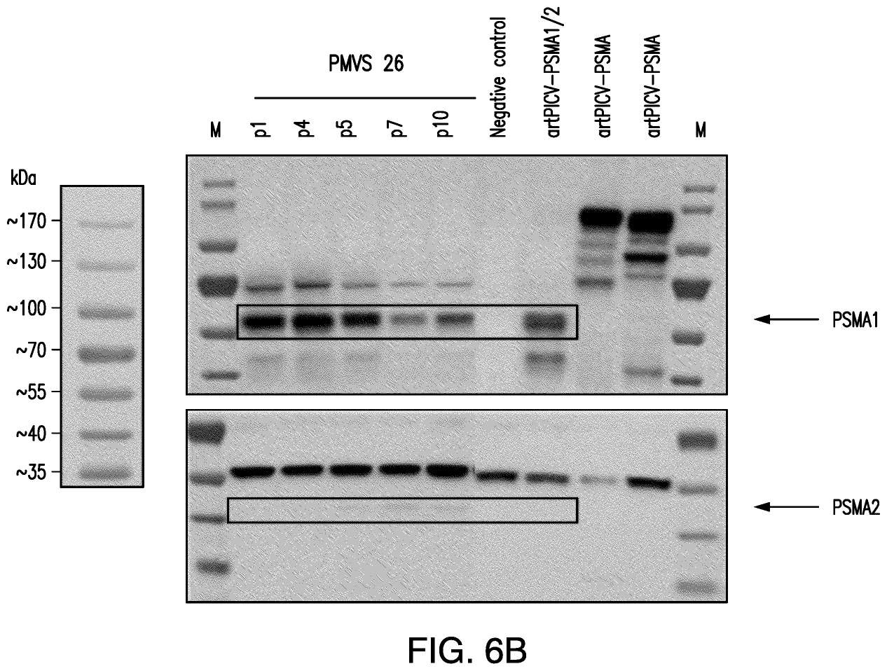

As shown by FIG. 6A, a PMVS, PMVS 26, stably expressed the encoded PSMA1 and PSMA2 transgenes up to passage level 10 without any transgene deletions in either segment. As shown by FIG. 6B, Western Blot analysis revealed that PSMA1 protein expression in PMVS 26 was detectable up to passage level 10. The protein expression results of PSMA2 were compromised by the poor quality of the antibody used, but sequencing of vector genome showed correct full length insert of coding sequence.

### 6.1.5 Vector Design and Transgene Stability of Fusion Transgene

Vectors with the following fusion transgene were tested: artLCMV-PAP_PSA-NP/PAP_PSA-GP, artLCMV-PSA_PAP-NP/PSA_PAP-GP, and artLCMV-PAP_PSA-NP/PSMA-GP. PSMA (full length) vector segment has 2253 bp which encodes 751 amino acids, and PAP_PSA fusion vector segment has 1941 bp, which encodes 647 amino acids. All three vectors were found to be unstable in P1.

6.2 Immunogenicity of artLCMV and artPICV Vectors Encoding PAP, PSA and PSMA in Mice

### 6.2.1 Immunogenicity of Single Vector Constructs and Vector Combinations

To analyze the ability of single vector constructs or combinations thereof to induce an immune response against the encoded antigens, intravenous immunization was performed in mice with the indicated vector constructs at 1×10⁵ RCV FFU/dose (see Table 2 Study Layout). Intracellular cytokine staining (ICS) using freshly isolated splenocytes was performed on day 7 post immunization. C57BL/6×Balb/c hybrid mice (i.e., CB6F1 mice) were used to cover H-2b and H-2d restricted T cell epitopes. A Tukey's multiple comparisons test using GraphPad Prism (one way ANOVA) was conducted for ICS data by comparing the means of all groups against each other. P values of p<0.05 (*), p<0.01 (**), p<0.005 (***) and p<0.001 (****) were considered significant.

TABLE 2
Study Layout

| group | Day 0 |
|---|---|
| 1 | buffer |
| 2 | artLCMV-PAP-NP/PSA-GP |
| 3 | artPICV-PAP-NP/PSA-GP |
| 4 | artLCMV-PSMA2-NP/PSMA1-GP |
| 5 | artPICV-PSMA1-NP/PSMA2-GP |
| 6 | artLCMV-PAP-NP/PSA-GP + artLCMV-PSMA2-NP/PSMA1-GP |
| 7 | artPICV-PAP-NP/PSA-GP + artPICV-PSMA1-NP/PSMA2-GP |

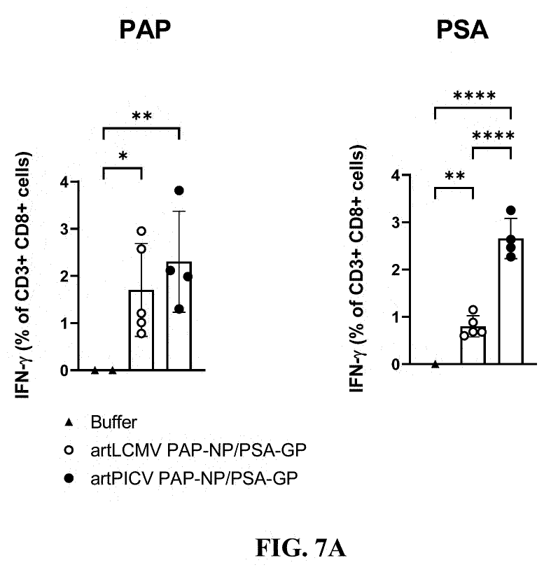

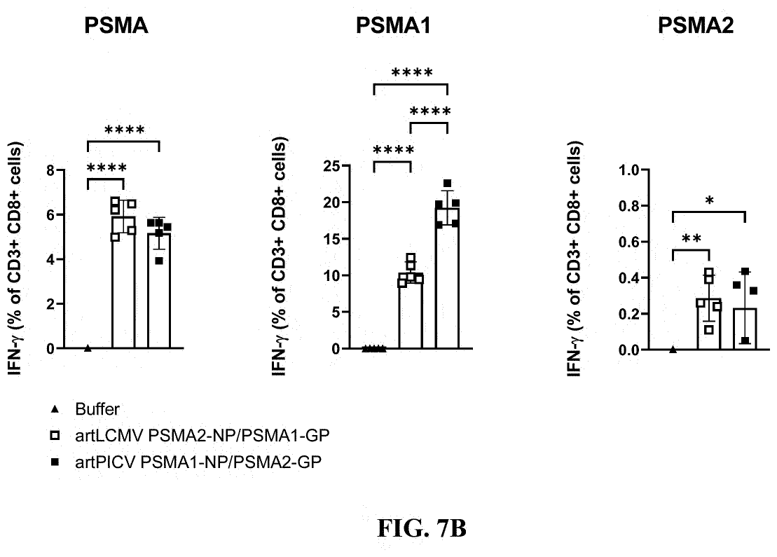

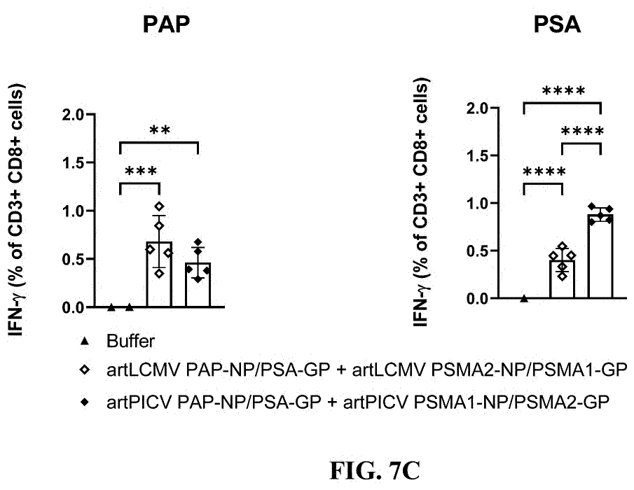

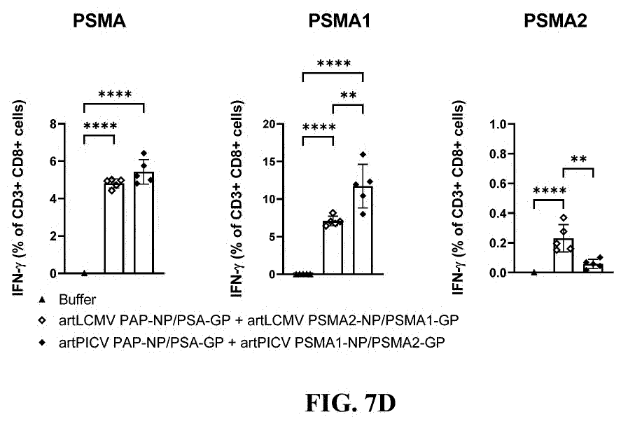

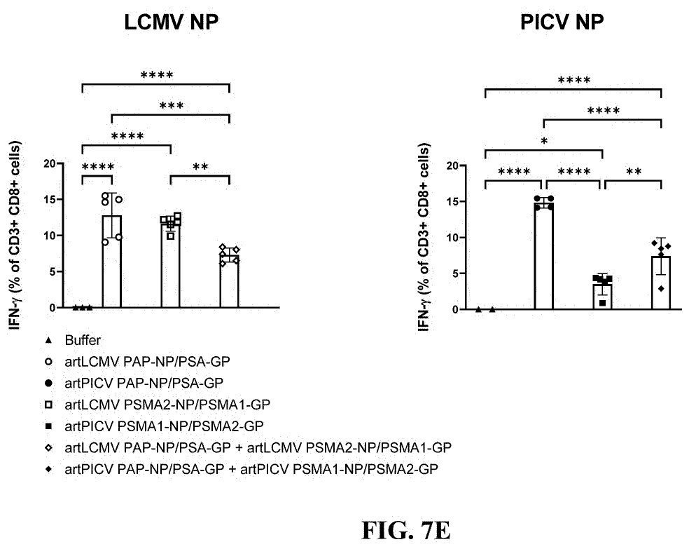

All tested vectors induced CD8 T cell responses against the encoded antigens after initial administration (FIGS. 7A and 7B). Both artLCMV-PAP-NP/PSA-GP (group 2) and artPICV-PAP-NP/PSA-GP (group 3) induced comparable PAP-specific T cell responses (FIG. 7A). However, induced T cell response to PSA were significantly higher in animals immunized with artPICV-PAP-NP/PSA-GP (group 3) compared to artLCMV-PAP-NP/PSA-GP (group 2) (FIG. 7A).

Similarly, comparable PSMA-specific T cell responses were observed after immunization with artPICV-PSMA1-NP/PSMA2-GP and artLCMV-PSMA2-NP/PSMA1-GP. However, analysis of responses directed to individual parts of the PSMA antigen revealed a significantly higher CD8 T cell response to PSMA1 after administration of artPICV-PSMA1-NP/PSMA2-GP (group 5) compared to artLCMV-PSMA2-NP/PSMA1-GP (group 4) (FIG. 7B).

To investigate whether mixed vectors would still induce T cell responses against all encoded antigens, mice in groups 6 and 7 were immunized with a combination of artLCMV-PAP-NP/PSA-GP and artLCMV-PSMA2-NP/PSMA1-GP (group 6) or artPICV-PAP-NP/PSA-GP and artPICV-PSMA1-NP/PSMA2-GP (group 7), respectively. As shown in FIGS. 7C and 7D, co-administration of a second vector did not abolish immunogenicity of the tested vectors and all vector constructs still induced considerable CD8 T cell responses against the encoded antigens after initial administration.

As shown in FIG. 7E, vector-backbone specific CD8 T cell responses were induced in all treatment groups. LCMV NP-specific T cell responses were significantly lower in animals of Group 6 immunized with a combination of artLCMV-PAP-NP/PSA-GP and artLCMV-PSMA2-NP/PSMA1-GP compared to mice of Group 2 or 4, immunized with the single vectors only. This was unexpected since the total titer of the viruses injected in the test animals was two times higher in the combination-vector group (artLCMV-PAP-NP/PSA-GP and artLCMV-PSMA2-NP/PSMA1-GP) than the single-vector group (artLCMV-PAP-NP/PSA-GP or artLCMV-PSMA2-NP/PSMA1-GP). In case of artPICV-based vectors, significantly higher vector backbone (PICV NP)-specific T cell responses were observed after administration of artPICV-PAP-NP/PSA-GP (Group 3) compared to immunization with artPICV-PSMA1-NP/PSMA2-GP (Group 5) and artPICV-PAP-NP/PSA-GP+artPICV-PSMA1-NP/PSMA2-GP (Group 7).

6.2.2 Immunogenicity of artLCMV-PAP-NP/PSA-GP and artPICV-PAP-NP/PSA-GP after Homologous or Heterologous Alternating Vector Administration

To analyze the immunogenicity of artLCMV-PAP-NP/PSA-GP and artPICV-PAP-NP/PSA-GP vectors after homologous or heterologous alternating vector administration, mice were immunized intravenously with 1×10⁵ RCV FFU/dose of artLCMV-PAP-NP/PSA-GP (groups 2 and 4) or artPICV-PAP-NP/PSA-GP (groups 3 and 5) on day 0. Three weeks later on day 21, animals in groups 2 and 5 were sequentially dosed with 1×10⁵ RCV FFU/dose of artLCMV-PAP-NP/PSA-GP, whereas mice in groups 3 and 4 were sequentially dosed with 1×10⁵ RCV FFU/dose of artPICV-PAP-NP/PSA-GP (see Table 3 Study Layout). Intracellular cytokine staining (ICS) using freshly isolated splenocytes was performed on day 26 to detect T cell responses specific for the encoded prostate cancer-related antigens PAP and PSA as well as the arenaviral vector backbone protein NP. A Tukey's multiple comparisons test using GraphPad Prism (one way ANOVA) was conducted for ICS data by comparing the means of all groups against each other. P values of p<0.05 (*), p<0.01 (**), p<0.005 (***) and p<0.001 (****) were considered significant.

TABLE 3
Study Layout

| group | Day 0 | Day 21 |
|---|---|---|
| 1 | buffer | buffer |
| 2 | artLCMV-PAP-NP/PSA-GP | artLCMV-PAP-NP/PSA-GP |
| 3 | artPICV-PAP-NP/PSA-GP | artPICV-PAP-NP/PSA-GP |
| 4 | artLCMV-PAP-NP/PSA-GP | artPICV-PAP-NP/PSA-GP |
| 5 | artPICV-PSMA1-NP/PSMA2-GP | artLCMV-PAP-NP/PSA-GP |

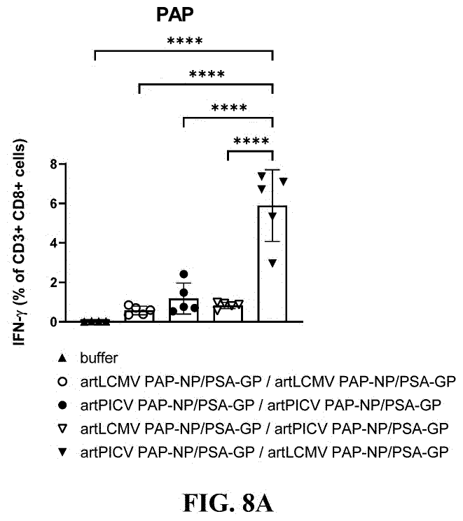

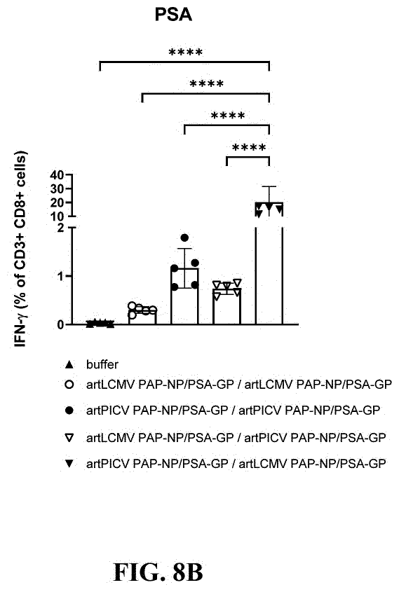

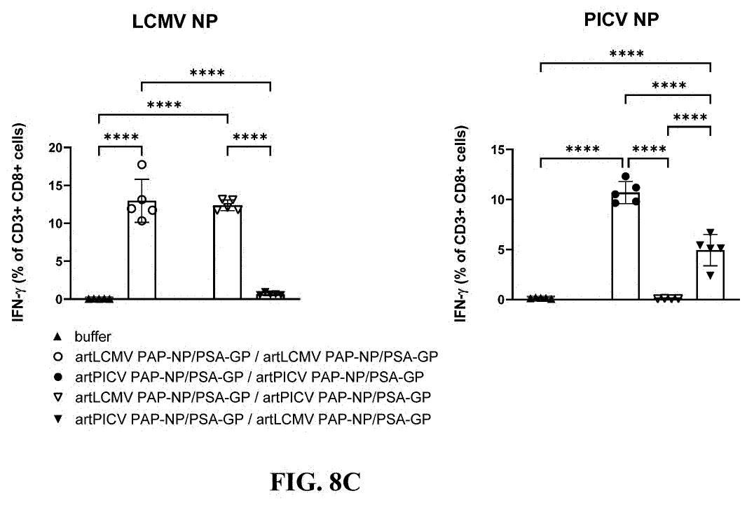

As shown in FIG. 8A and FIG. 8B, PAP- and PSA-specific CD8 T cell responses were detected in all test groups. However, animals of group 5, which were initially immunized with an artPICV-PAP-NP/PSA-GP vector and sequentially dosed with an artLCMV-PAP-NP/PSA-GP vector showed significantly higher PAP- (FIG. 8A) and PSA-specific (FIG. 8B) CD8 T cell responses compared to mice in all other test groups.

In case of PSA-specific T cell responses (FIG. 8B), there was a difference between groups 2 and 3, with considerably higher PSA-specific CD8 T cell responses induced after homologous alternating vector administration with artPICV-PAP-NP/PSA-GP (group 3) compared to artLCMV-PAP-NP/PSA-GP (group 2). Besides, there was a trend towards increase in PSA-specific CD8 T cell responses when animals initially dosed with artLCMV-PAP-NP/PSA-GP were sequentially dosed with the heterologous artPICV-PAP-NP/PSA-GP vector (group 4) compared to homologous alternating vector administration with artLCMV-PAP-NP/PSA-GP only (group 2).

Analysis of arenaviral NP-specific T cell responses (FIG. 8C) demonstrated the induction of CD8 T cell responses directed against the vector backbone that was used for initial administration. In case of initial administration of artLCMV-PAP-NP/PSA-GP there was no significant difference in the LCMV NP-specific T cell responses between group 2 (i.e., after homologous sequential administration with artLCMV-PAP-NP/PSA-GP) and group 4 (i.e., after heterologous sequential administration with artPICV-PAP-NP/PSA-GP). In contrast, when mice were initially dosed with artPICV-PAP-NP/PSA-GP, PICV NP-specific T cell responses were significantly lower when animals were sequentially dosed with the heterologous artLCMV-PAP-NP/PSA-GP vector (group 5) compared to sequential homologous dosing with artPICV-PAP-NP/PSA-GP (group 3). Thus, the ratio of transgene- to vector-specific T cells was highest in group 5, i.e., after initially dosing with artPICV-PAP-NP/PSA-GP and sequentially dosing with artLCMV-PAP-NP/PSA-GP.

6.2.3 Immunogenicity of artLCMV-PSMA2-NP/PSMA1-GP and artPICV-PSMA1-NP/PSMA2-GP after Homologous or Heterologous Alternating Vector Administration

To analyze the immunogenicity of PSMA-expressing vectors artLCMV-PSMA2-NP/PSMA1-GP and artPICV-PSMA1-NP/PSMA2-GP after homologous or heterologous alternating vector administration, mice were immunized intravenously with 1×10⁵ RCV FFU/dose of artLCMV-PSMA2-NP/PSMA1-GP (groups 2 and 4) or artPICV-PSMA1-NP/PSMA2-GP (groups 3 and 5) on day 0. Three weeks later on day 21, animals in groups 3 and 4 were sequentially dosed with 1×10⁵ RCV FFU/dose of artLCMV-PSMA2-NP/PSMA1-GP, whereas mice in groups 2 and 5 were sequentially dosed with 1×10⁵ RCV FFU/dose of artPICV-PSMA1-NP/PSMA2-GP (see Table 4 Study Layout). Intracellular cytokine staining (ICS) using freshly isolated splenocytes was performed on day 26 to detect T cell responses specific for the encoded prostate cancer-related antigen PSMA as well as the arenaviral vector backbone protein NP. A Tukey's multiple comparisons test using GraphPad Prism (one way ANOVA) was conducted for ICS data by comparing the means of all groups against each other. P values of p<0.05 (*), p<0.01 (**), p<0.005 (***) and p<0.001 (****) were considered significant.

TABLE 4
Study Layout

| group | Day 0 | Day 21 |
|---|---|---|
| 1 | buffer | buffer |
| 2 | artLCMV-PSMA2-NP/ | artLCMV-PSMA2-NP/ |
| | PSMA1-GP | PSMA1-GP |
| 3 | artPICV-PSMA1-NP/ | artPICV-PSMA1-NP/ |
| | PSMA2-GP | PSMA2-GP |
| 4 | artLCMV-PSMA2-NP/ | artPICV-PSMA1-NP/ |
| | PSMA1-GP | PSMA2-GP |
| 5 | artPICV-PSMA1-NP/ | artLCMV-PSMA2-NP/ |
| | PSMA2-GP | PSMA1-GP |

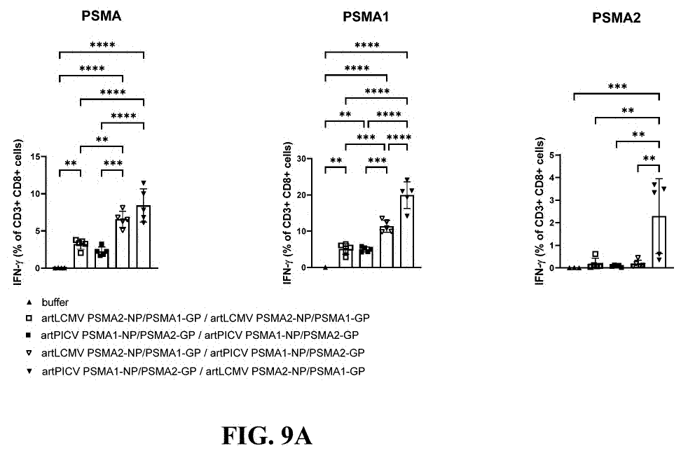

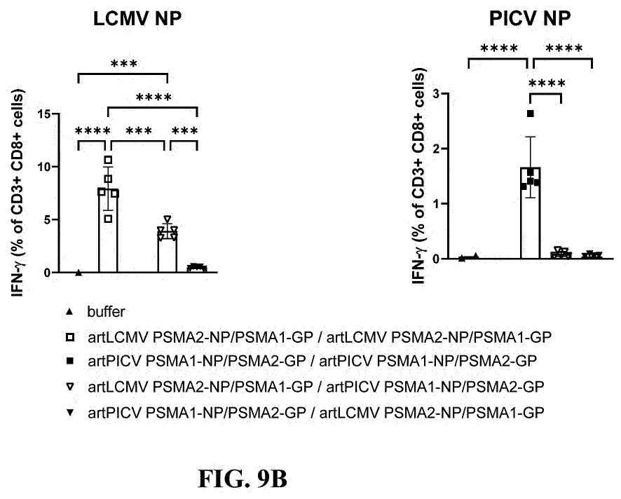

PSMA-specific CD8 T cell responses were detected in all test groups (FIG. 9A). However, highest antigen-specific responses directed against both parts of the PSMA antigen (i.e., PSMA1 and PSMA2) were observed in animals of group 5, initially dosed with artPICV-PSMA1-NP/PSMA2-GP and sequentially dosed with artLCMV-PSMA2-NP/PSMA1-GP.

In contrast, as shown in FIG. 9B, arenaviral NP-specific T cell responses (FIG. 9B) were significantly higher after homologous alternating vector administration with either artLCMV-PSMA2-NP/PSMA1-GP (group 2) or artPICV-PSMA1-NP/PSMA2-GP (group 3) compared to heterologous alternating vector administration using sequential administration of artLCMV-PSMA2-NP/PSMA1-GP followed by artPICV-PSMA1-NP/PSMA2-GP (group 4) or artPICV-PSMA1-NP/PSMA2-GP followed by artLCMV-PSMA2-NP/PSMA1-GP (group 5). Thus, the ratio of transgene- to vector-specific T cells was highest in group 5, i.e., after initially dosing with artPICV-PSMA1-NP/PSMA2-GP and sequentially dosing with artLCMV-PSMA2-NP/PSMA1-GP.

6.2.4 Immunogenicity of artLCMV and artPICV Vector Combinations after Homologous or Heterologous Alternating Vector Administration

The ability of individual vector constructs to induce an immune response against the encoded antigens was analyzed after administration of vector mixes (i.e., artLCMV-PAP-NP/PSA-GP+artLCMV-PSMA2-NP/PSMA1-GP; artPICV-PAP-NP/PSA-GP+artPICV-PSMA1-NP/PSMA2-GP) using both homologous and heterologous alternating vector administration.

Mice in all groups were initially dosed on day 0 and sequentially dosed 21 days later by intravenous administration of premixed vectors at 1×10⁵ RCV FFU/vector. Mice in groups 1 and 3 were first immunized with a combination of artLCMV-PAP-NP/PSA-GP and artLCMV-PSMA2-NP/PSMA1-GP (i.e., artLCMV vector mix). Animals in group 1 were sequentially dosed with the same vector combination, whereas mice in group 3 were sequentially dosed with a combination of artPICV-PAP-NP/PSA-GP and artPICV-PSMA1-NP/PSMA2-GP (i.e., artPICV vector mix). Animals in groups 2 and 4 received a first dose of the artPICV vector mix. Mice in group 2 were subsequently sequentially dosed homologously using the same artPICV vector mix for the second administration. In contrast, animals in group 4 were sequentially dosed with the artLCMV vector mix (see Table 5 Study Layout). Intracellular cytokine staining (ICS) using freshly isolated splenocytes was performed on day 26 to detect T cell responses specific for the encoded prostate cancer-related antigens PAP, PSA and PSMA as well as the arenaviral vector backbone protein NP. A Tukey's multiple comparisons test using GraphPad Prism (one way ANOVA) was conducted for ICS data by comparing the means of all groups against each other. P values of p<0.05 (*), p<0.01 (**), p<0.005 (***) and p<0.001 (****) were considered significant.

TABLE 5
Study Layout

| group | Day 0 | Day 21 |
|---|---|---|
| 1 | artLCMV PAP-NP/ | artLCMV PAP-NP/ |
| | PSA-GP + artLCMV | PSA-GP + artLCMV |
| | PSMA2-NP/PSMA1-GP | PSMA2-NP/PSMA1-GP |
| 2 | artPICV PAP-NP/ | artPICV PAP-NP/ |
| | PSA-GP + artPICV | PSA-GP + artPICV |
| | PSMA1-NP/PSMA2-GP | PSMA1-NP/PSMA2-GP |
| 3 | artLCMV PAP-NP/ | artPICV PAP-NP/ |
| | PSA-GP + artLCMV | PSA-GP + artPICV |
| | PSMA2-NP/PSMA1-GP | PSMA1-NP/PSMA2-GP |
| 4 | artPICV PAP-NP/ | artLCMV PAP-NP/ |
| | PSA-GP + artPICV | PSA-GP + artLCMV |
| | PSMA1-NP/PSMA2-GP | PSMA2-NP/PSMA1-GP |

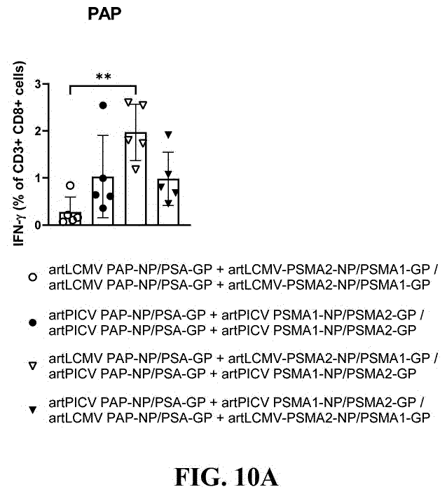

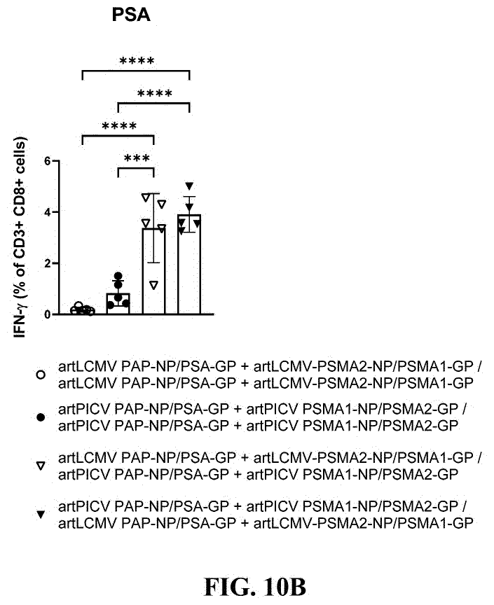

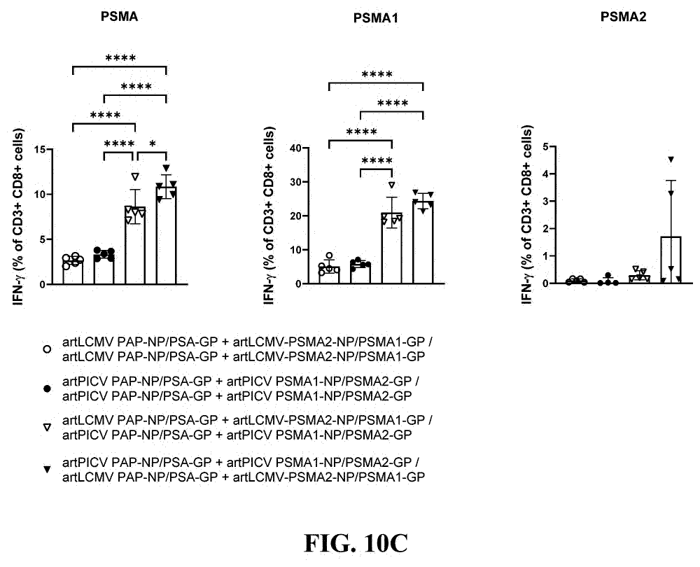

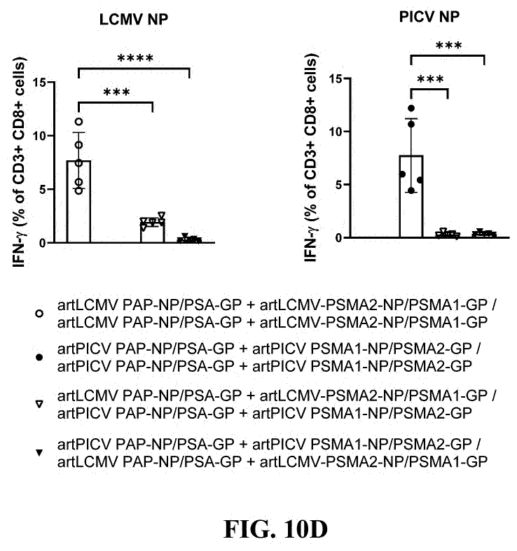

As shown in FIG. 10A, PAP-specific CD8 T cell responses were detected in all test groups. Highest PAP-specific CD8 T cell responses were observed in animals of group 3, which were initially dosed with the artLCMV vector mix and sequentially dosed with the artPICV vector mix. This was in significant contrast to animals of group 1 which were also initially dosed with the artLCMV vector mix but sequentially dosed in a homologous manner with the same artLCMV vector mix. Respective animals demonstrated the lowest PAP-specific CD8 T cell responses of all test groups.

Heterologous alternating vector administration was also significantly superior to homologous procedure in the induction of PSA-specific CD8 T cell responses. As shown in FIG. 10B, highest PSA-specific CD8 T cell responses were observed in animals of groups 3 and 4, which were initially dosed with the artLCMV vector mix and sequentially dosed with the artPICV vector mix (group 3) or initially dosed with the artPICV vector mix and sequentially dosed with the artLCMV vector mix (group 4). Significantly lower T cell response to PSA were induced in animals immunized twice with the same artLCMV (group 1) or artPICV (group 2) vector mix, respectively. A comparison between these groups with homologous alternating vector administration revealed higher PSA-specific CD8 T cell responses in animals of group 2, treated with the artPICV vector mix, compared to animals of group 1, which were immunized with the artLCMV vector mix.

The analysis of PSMA-specific CD8 T cell responses induced in the different test groups further substantiated the observation of superior immunogenicity after heterologous alternating vector administration. As shown in FIG. 10C, highest PSMA-specific CD8 T cell responses were observed in animals of groups 3 and 4, which were initially dosed with the artLCMV vector mix and sequentially dosed with the artPICV vector mix (group 3) or initially dosed with the artPICV vector mix and sequentially dosed with the artLCMV vector mix (group 4). Significantly lower T cell responses to PSMA were induced in animals treated with homologous alternating vector administration i.e., being immunized twice with the same artLCMV (group 1) or artPICV (group 2) vector mix, respectively. This observation was consistent, independent of whether the immune response to the entire PSMA antigen or only individual subdomains thereof were analyzed.

In contrast, vector backbone-specific immune responses were significantly higher after homologous alternating vector administration of either artLCMV vector mix (group 1) or artPICV vector mix (group 2) compared to heterologous alternating vector administration using sequential administration of artLCMV vector mix followed by artPICV vector mix (group 3) or artPICV vector followed by artLCMV vector mix (group 4), as demonstrated by vector NP-specific T cell responses (FIG. 10D). Thus, the ratio of transgene- to vector-specific T cells was highest in group 4, i.e., after initially dosing with artPICV vector mix and sequentially dosing with artLCMV vector mix.

### 6.3 Treating Prostate Cancer Patients

### 6.3.1 Viral Vector-Based Therapeutic Immunotherapies

Four individual viral vector-based therapeutic immunotherapies, as described in Table 6. Descriptions of the Viral Vector-Based Therapeutic Immunotherapies below, are studied, exploring two treatment regimens:
Group 1: Patients receive the alternating 2-vector treatment:
artPICV-PAP-NP/PSA-GP is administered first in alternating sequence with artLCMV-PAP-NP/PSA-GP.
Group 2: Patients receive the alternating 4-vector treatment:
artPICV-PAP-NP/PSA-GP and artPICV-PSMA1-NP/PSMA2-GP is administered first in alternating sequence with artLCMV-PAP-NP/PSA-GP and artLCMV-PSMA2-NP/PSMA1-GP.

TABLE 6
Descriptions of the Viral Vector-Based Therapeutic Immunotherapies

| | Vector | Antigen(s) | |
|---|---|---|---|
| IMP Name | backbone | expressed | Description |
| artLCMV- | LCMV | PAP & PSA | A genetically engineered LCMV |
| PAP-NP/ | | | vector based on the tri-segmented, |
| PSA-GP | | | replicating arenavirus vector |
| | | | technology, based on the LCMV |
| | | | strain Clone 13 with the viral |
| | | | surface glycoprotein from LCMV |
| | | | strain WE, expressing prostate |
| | | | cancer-related antigens PAP and PSA. |
| artLCMV- | LCMV | PSMA | A genetically engineered LCMV |
| PSMA2-NP/ | | | vector based on the tri-segmented, |
| PSMA1-GP | | | replicating arenavirus vector |
| | | | technology, based on the LCMV |
| | | | strain Clone 13 with the viral |
| | | | surface glycoprotein from LCMV |
| | | | strain WE, expressing a prostate |
| | | | cancer-related antigen PSMA. |
| artPICV- | PICV | PAP & PSA | A genetically engineered PICV |
| PAP-NP/ | | | vector based on the tri-segmented, |
| PSA-GP | | | replicating arenavirus vector |
| | | | technology, based on the PICV |
| | | | strain p18, expressing PAP/PSA. |
| artPICV- | PICV | PSMA | A genetically engineered PICV |
| PSMA1-NP/ | | | vector based on the tri-segmented, |
| PSMA2-GP | | | replicating arenavirus vector |
| | | | technology, based on the PICV |
| | | | strain p18, expressing PSMA. |

Abbreviations:
LCMV = lymphocytic choriomeningitis virus,
PAP = prostatic acid phosphatase,
PSA = prostate-specific antigen,
PICV = pichinde virus,
PSMA = prostate-specific membrane antigen

### 6.3.2 Treatment Overview

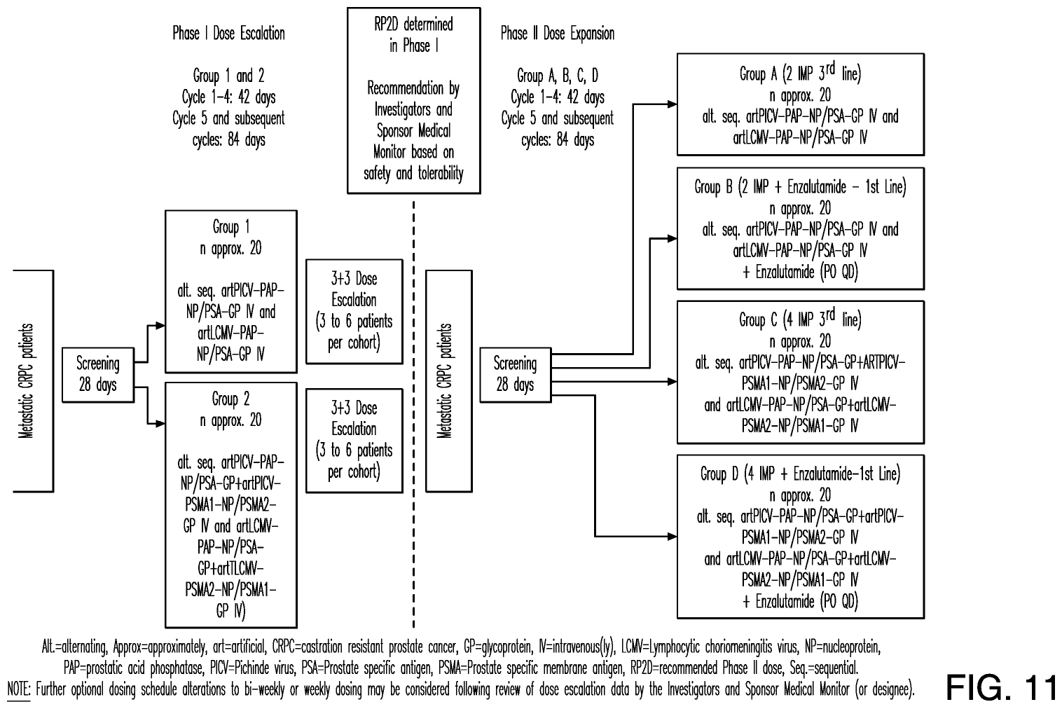

Adult patients with mCRPC are treated in two parts: Phase 1 Dose Escalation and Phase 2 Dose Expansion. Schematics of the study design are presented in FIG. 11.

### (i) Phase I Dose Escalation

Phase I Dose Escalation has two treatment groups:
Group 1: Alternating 2-vector treatment. artPICV-PAP-NP/PSA-GP is administered first in alternating sequence with artLCMV-PAP-NP/PSA-GP.
Group 2: Alternating 4-vector treatment. artPICV-PAP-NP/PSA-GP & artPICV-PSMA1-NP/PSMA2-GP is administered first in alternating sequence with artLCMV-PAP-NP/PSA-GP & artLCMV-PSMA2-NP/PSMA1-GP.

Study treatment is administered as indicated in Section 6.3.5(i).

### (ii) Phase II Dose Expansion

Phase II Dose Expansion commences upon completion of the Phase I Dose Escalation.

Study treatment regimen will be based on the safety, efficacy, biomarker, and immunogenicity results from the Phase I Dose Escalation portion of the study as indicated in Section 6.3.5(ii).

### 6.3.3 Patient Population: Inclusion Criteria

### For all Patients:

Patients who meet all of the following criteria is eligible to participate in the study:
1. Male patients≥18 years of age at the time of signing the Informed Consent Form (ICF).
2. Willing and able to give voluntary informed consent for participation in the study.
3. Histologically confirmed diagnosis of prostate adenocarcinoma without neuroendocrine differentiation or small cell features.
4. Documented castration condition with serum testosterone levels of <50 ng/dL (1.7 nmol/L). The castration condition can be obtained by bilateral orchiectomy or use of luteinizing hormone-releasing hormone (LHRH) analog (agonist or antagonist). Patients who have not undergone surgical bilateral orchiectomy must be willing to continue LHRH analog during the course of the study.
5. Patients must have ≥1 measurable soft tissue lesion by computed tomography (CT) and/or magnetic resonance imaging (MRI), which is assessed for tumor response following Response Evaluation Criteria in Solid Tumors (RECIST) v1.1 and immune RECIST (iRECIST) during study conduct.
6. Patients must have had disease progression on standard of care therapy assessed. Disease progression is defined by one or more of the following criteria according to Prostate Cancer Clinical Trials Working Group 3 (PCWG3) guidance:
   For patients who manifest disease progression solely as arising PSA level, PCWG3 requires at least two consecutive rising PSA values with ≥1 week apart (not limited to the 28-day screening period) and a minimum starting value of 1.0 ng/mL. Most recent PSA level must be obtained within 21 days prior to first study drug treatment. (Note: For patients receiving flutamide, at least one of the PSA values must be obtained ≥4 weeks after flutamide discontinuation. For patients receiving bicalutamide or nilutamide, at least one of the PSA values must be obtained ≥6 week after antiandrogen discontinuation.)
   For patients with measurable nodal or visceral lesions, disease progression of one of these lesions define by RECIST 1.1 is sufficient for eligibility independent of PSA. In case of lymph node ≥15 mm in diameter, it is considered measurable and used to evaluate change of size.
   For patients with bone metastases, progression is defined by the appearance of ≥2 new lesions by bone scan or other scans (e.g. MRI)
7. Antiandrogen withdrawal followed by progression must take place at least ≥4 weeks before enrollment unless case-specific exceptions are applied. LHRH agonists or antagonists should be continued.
8. Unless case-specific exceptions are applied, patients must have:
   An archival soft tissue tumor specimen collected following the patients' progression from last treatment or the ability to provide fresh soft tissue biopsy specimen prior to dosing. If archival sample is provided, the sample must not be older than 2 years.
   A soft tissue lesion potentially available for biopsy on-study.
9. Eastern Cooperative Oncology Group (ECOG) performance status of 0 to 1.
10. Prior curative radiation therapy must have been completed at least 4 weeks prior to study drug administration. Prior focal palliative radiotherapy must have been completed at least 2 weeks prior to study drug administration.
11. Screening laboratory values must meet the following criteria and should be obtained within 28 days prior to study treatment administration:
   Absolute neutrophil count≥1,500/mm³ (1.5×10⁹/L)
   Platelets≥100×10³/mm³ (100×10⁹/L)
   Hemoglobin≥8.5 g/dL
   Serum creatinine≤2.0×upper limit of normal (ULN) or creatinine clearance >30 mL/min (using the Cockcroft-Gault formula)
   Aspartate aminotransferase/alanine aminotransferase≤3×ULN
   Total bilirubin≤1.5×ULN (except patients with Gilbert's syndrome, who can have total bilirubin<3.0 mg/dL)

### 6.3.4 Dose Escalation Guidelines—Provisional Dose Levels Explored

artLCMV-PAP-NP/PSA-GP, artPICV-PAP-NP/PSA-GP, artLCMV-PSMA2-NP/PSMA1-GP, and artPICV-PSMA1-NP/PSMA2-GP, are administered IV (as an IV push or infusion). A starting dose of 1×10⁷ RCV FFU per dose per patient for each vector is administered. Furthermore, a dose escalation plan for the Phase 1 portion of the clinical study permits dose increments of up to one log order between cohorts (i.e., 10⁷, 10⁸, 10⁹ RCV FFU).

For Phase 1 Dose Escalation Group 1 and 2, the proposed human starting dose of artLCMV-PAP-NP/PSA-GP, artPICV-PAP-NP/PSA-GP, artLCMV-PSMA2-NP/PSMA1-GP, and artPICV-PSMA1-NP/PSMA2-GP is 1×10⁷ RCV FFU. A non-limiting example of potential dose escalation is given in Table 7. Provisional Dose Level for Group 1 (Alternating 2-Vector Treatment of artPICV-PAP-NP/PSA-GP and artLCMV-PAP-NP/PSA-GP) and Table 8. Provisional Dose Level for Group 2 (Alternating 4-Vector Treatment of artPICV-PAP-NP/PSA-GP+artPICV-PSMA1-NP/PSMA2-GP and artLCMV-PAP-NP/PSA-GP+artLCMV-PSMA2-NP/PSMA1-GP).

TABLE 7
Provisional Dose Level for Group 1 (Alternating 2-Vector Treatment
of artPICV-PAP-NP/PSA-GP and artLCMV-PAP-NP/PSA-GP)

| | artPICV-PAP-NP/PSA-GP | artLCMV-PAP-NP/PSA-GP |
|---|---|---|
| Level | Provisional Dose | Provisional Dose |
| −1 | 1 × 10⁶ RCV FFU | 1 × 10⁶ RCV FFU |
| 1 | 1 × 10⁷ RCV FFU | 1 × 10⁷ RCV FFU |
| (starting | | |
| dose) | | |
| 2 | 1 × 10⁸ RCV FFU | 1 × 10⁸ RCV FFU |
| 3 | 1 × 10⁹ RCV FFU | 1 × 10⁹ RCV FFU |

Abbreviations:
FFU = focus-forming units,
RCV = replication-competent virus

TABLE 8
Provisional Dose Level for Group 2 (Alternating 4-Vector Treatment
of artPICV-PAP-NP/PSA-GP + artPICV-PSMA1-NP/PSMA2-GP
and artLCMV-PAP-NP/PSA-GP + artLCMV-PSMA2-NP/PSMA1-GP)

| | artPICV-PAP-NP/ | artPICV-PSMA1-NP/ | artLCMV-PAP-NP/ | artLCMV-PSMA2-NP/ |
|---|---|---|---|---|
| Level | PSA-GP | PSMA2-GP | PSA-GP | PSMA1-GP |
| −1 | 1 × 10⁶ RCV FFU | 1 × 10⁶ RCV FFU | 1 × 10⁶ RCV FFU | 1 × 10⁶ RCV FFU |
| 1 | 1 × 10⁷ RCV FFU | 1 × 10⁷ RCV FFU | 1 × 10⁷ RCV FFU | 1 × 10⁷ RCV FFU |
| (starting | | | | |
| dose) | | | | |
| 2 | 1 × 10⁸ RCV FFU | 1 × 10⁸ RCV FFU | 1 × 10⁸ RCV FFU | 1 × 10⁸ RCV FFU |
| 3 | 1 × 10⁹ RCV FFU | 1 × 10⁹ RCV FFU | 1 × 10⁹ RCV FFU | 1 × 10⁹RCV FFU |

Abbreviations:
FFU = focus-forming units,
RCV = replication-competent virus

### 6.3.5 Treatment Regimen and Cycle Duration

Patients are treated until they experience unacceptable treatment-related toxicity, disease progression (immune confirmed progressive disease (iCPD) per iRECIST or bone progression per PCWG3) or withdraw consent.

### (i) Phase I Dose Escalation

For Group 1 (alternating 2-vector treatment of artPICV-PAP-NP/PSA-GP and artLCMV-PAP-NP/PSA-GP):
artPICV-PAP-NP/PSA-GP and artLCMV-PAP-NP/PSA-GP are given in alternating IV administrations. artPICV-PAP-NP/PSA-GP is administered first, followed by artLCMV-PAP-NP/PSA-GP.
For Cycles 1 and 2, a treatment cycle is defined as a period of 42 days. artPICV-PAP-NP/PSA-GP is administered first, followed by artLCMV-PAP-NP/PSA-GP, alternating treatment every three weeks (21 days) for the first four administrations.
   artPICV-PAP-NP/PSA-GP is administered IV on Day 1 of Cycles 1 and 2.
   artLCMV-PAP-NP/PSA-GP is administered IV on Day 22 of Cycles 1 and 2.
For Cycle 3 and subsequent cycles, a treatment cycle is defined as a period of 84 days. Cycle 3 Day 1 starts following the completion of Cycle 2 Day 42. artPICV-PAP-NP/PSA-GP and artLCMV-PAP-NP/PSA-GP dose administrations in Cycle 3 and subsequent cycles have a time window of 7 days. artPICV-PAP-NP/PSA-GP and artLCMV-PAP-NP/PSA-GP doses alternate every six weeks (42 days) as follows:
   artPICV-PAP-NP/PSA-GP is administered IV on Day 1 of Cycle 3 and subsequent cycles.
   artLCMV-PAP-NP/PSA-GP is administered IV on Day 43 of Cycle 3 and subsequent cycles.

For Group 2 (alternating 4-vector treatment of artPICV-PAP-NP/PSA-GP+artPICV-PSMA1-NP/PSMA2-GP and artLCMV-PAP-NP/PSA-GP+artLCMV-PSMA2-NP/PSMA1-GP):
artPICV-PAP-NP/PSA-GP+artPICV-PSMA1-NP/PSMA2-GP and artLCMV-PAP-NP/PSA-GP+artLCMV-PSMA2-NP/PSMA1-GP are given in alternating IV administrations. artPICV-PAP-NP/PSA-GP+artPICV-PSMA1-NP/PSMA2-GP are administered first, then followed by artLCMV-PAP-NP/PSA-GP+artLCMV-PSMA2-NP/PSMA1-GP.
For Cycles 1 and 2, a treatment cycle is defined as a period of 42 days. artPICV-PAP-NP/PSA-GP+artPICV-PSMA1-NP/PSMA2-GP are administered first, followed by artLCMV-PAP-NP/PSA-GP+artLCMV-PSMA2-NP/PSMA1-GP, alternating treatment every three weeks (21 days) for the first four administrations.
   artPICV-PAP-NP/PSA-GP+artPICV-PSMA1-NP/PSMA2-GP are administered IV on Day 1 of Cycles 1 and 2.
   artLCMV-PAP-NP/PSA-GP+artLCMV-PSMA2-NP/PSMA1-GP are administered IV on Day 22 of Cycles 1 and 2.
For Cycle 3 and subsequent cycles, a treatment cycle is defined as a period of 84 days. Cycle 3 Day 1 starts following the completion of Cycle 2 Day 42. artPICV-PAP-NP/PSA-GP+artPICV-PSMA1-NP/PSMA2-GP and artLCMV-PAP-NP/PSA-GP+artLCMV-PSMA2-NP/PSMA1-GP dose administrations in Cycle 3 and subsequent cycles have a time window of 7 days. artPICV-PAP-NP/PSA-GP+artPICV-PSMA1-NP/PSMA2-GP and artLCMV-PAP-NP/PSA-GP+artLCMV-PSMA2-NP/PSMA1-GP doses alternate every six weeks (42 days) as follows:
   artPICV-PAP-NP/PSA-GP+artPICV-PSMA1-NP/PSMA2-GP are administered IV on Day 1 of Cycle 3 and subsequent cycles.
   artLCMV-PAP-NP/PSA-GP+artLCMV-PSMA2-NP/PSMA1-GP are administered IV on Day 43 of Cycle 3 and subsequent cycles.

For Phase I Dose Escalation, administration schedules for artPICV-PAP-NP/PSA-GP and artLCMV-PAP-NP/PSA-GP regimen and/or artPICV-PAP-NP/PSA-GP+artPICV-PSMA1-NP/PSMA2-GP and artLCMV-PAP-NP/PSA-GP+artLCMV-PSMA2-NP/PSMA1-GP regimen may be modified for the next cohort based on safety, efficacy, or biomarker data.

For Phase I Dose Escalation, the following information are collected: (1) Incidence of DLTs from the first study drug administered during the DLT observation period; (2) Safety: type, frequency, and severity of AEs and SAEs; (3) Tolerability: dose interruptions, reductions and dose intensity, and evaluations of laboratory values; (4) ORR, DCR, PFS, OS and duration of response using RECIST v1.1/iRECIST (soft tissues) and PCWG3 (bone) criteria; (5) Proportion of patients with PSA response (≥50% decrease in PSA from baseline to the lowest postbaseline PSA result, confirmed by a second consecutive PSA assessment at least 3 weeks later); (6) Best PSA response at any time; (7) Measurement of antigen (PAP, PSA, and/or PSMA)-specific T cell responses, antigen-specific T cell functionality assays; (8) Characterization of immune cells (immunophenotyping) CD4 and CD8 T cell measurements; and (9) Assessment of biomarkers in tumor specimens, blood, and serum/plasma.

### (ii) Phase II Dose Expansion

Treatment regimen and cycle duration of the arenaviral vectors are the same as described in Phase I.

In some treatment groups patients will receive a combination therapy of a medication that is approved for treatment of advanced prostate cancer together with artLCMV-PAP-NP/PSA-GP, artPICV-PAP-NP/PSA-GP, artLCMV-PSMA2-NP/PSMA1-GP, and artPICV-PSMA1-NP/PSMA2-GP.

For Phase II Dose Expansion, the following information is collected: (1) ORR and DCR using RECIST v1.1/iRECIST (soft tissues) and PCWG3 (bone) criteria; (2) Proportion of patients with PSA response (≥50% decrease in PSA from baseline to the lowest post-baseline PSA result, confirmed by a second consecutive PSA assessment at least 3 weeks later); (3) Best PSA response at any time; (4) Tumor responses is assessed using RECIST v.1./iRECIST (soft tissue), PCWG3 (bone) criteria, such as duration of response, PFS, and OS; (5) Safety: type, frequency, and severity of AEs and SAEs; (6) Tolerability: dose interruptions, reductions and dose intensity, and evaluations of laboratory values; (7) Measurement of antigen (PAP, PSA, and/or PSMA)-specific T cell responses, antigen-specific T cell functionality assays; (8) Characterization of immune cells (immunophenotyping) CD4 and CD8 T cell measurements; (9) Assessment of biomarkers in tumor specimens, blood, and serum/plasma.

### 6.3.6 Efficacy Variables

For all groups of Phase 1 Dose Escalation and Phase 2 Dose Expansion, efficacy assessments include:
PSA levels tested every 3 weeks starting at Cycle 1 Day 1
Imaging scans for soft tissues and bone performed every 8 weeks for the first 24 weeks (i.e. starting from Cycle 2 Day 15, then Cycle 3 Day 29 and Day 84), then every 12 weeks starting from Cycle 4 Day 84 and onward.

Efficacy is assessed using PSA response according to PCWG3 (Scher et al. 2016), bone response according to PCWG3, and soft tissue response according to RECIST v1.1 (primary efficacy endpoint) and iRECIST (secondary efficacy endpoints). Bone progression requires confirmation by a second bone scan at least 6 weeks later.

Table 9. PCWG3 Criteria of Progression by Disease Manifestation shows assessments and progression criteria on the basis of changes in PSA, bone metastases, and measurable disease (Scher et al., 2016, J Clin Oncol, 34: 1402-18)

TABLE 9
PCWG3 Criteria of Progression by Disease Manifestation

| | Evaluation Criteria |
|---|---|
| Assessment | for Disease Progression |
| PSA | Obtain sequence of rising values at a |
| | minimum of 1-week intervals |
| | 1.0 ng/mL minimal starting value if |
| | confirmed rise is only indication of |
| | progression unless pure small-cell |
| | carcinoma |
| | Estimate pre-therapy PSA-DT if ≥3 |
| | values available ≥4 weeks apart |
| Target | Nodal or visceral progression sufficient |
| lesions | for trial entry independent of PSA |
| | Measurable lesions not required for entry |
| | Use RECIST v1.1 to record soft-tissue |
| | (nodal and visceral) lesions as target |
| | or nontarget |
| | Previously normal (<1.0 cm) lymph nodes |
| | must have grown by ≥5 mm in the short |
| | axis from baseline or nadir and be ≥1.0 cm |
| | in the short axis to be considered to have |
| | progressed |
| | If the node progresses to ≥1.5 cm in the |
| | short axis, it is measurable; nodes that |
| | have progressed to 1.0 to <1.5 cm are |
| | pathologic, subject to clinical discretion, |
| | and non-measurable |
| | For existing pathologic adenopathy, |
| | progression is defined per RECIST v1.1 |
| | Record presence of nodal and/or visceral |
| | disease separately: |
| | Nodal sites: Locoregional: pelvic only; |
| | Extrapelvic: retroperitoneal, mediastinal, |
| | thoracic, or other |
| | Visceral sites: lung, liver, adrenal, CNS |
| Prostate | Record prior treatment of primary tumor |
| | Perform directed pelvic imaging (CT, MRI, |
| | PET/CT, endorectal MRI, transrectal ultrasound) |
| | to document presence or absence of disease |
| Bone | Progression = appearance of ≥2 or more new |
| | lesions |
| | Confirm ambiguous results by other imaging |
| | modalities (e.g., CT or MRI), but only |
| | positivity on the bone scan defines |
| | metastatic disease to bone |

Abbreviations:
CNS = central nervous system;
PSA = prostate-specific antigen;
CT = computed tomography;
MRI = magnetic resonance imaging;
PCWG3 = Prostate Cancer Clinical Trials Working Group 3;
PET = positron emission tomography;
PSA-DT = PSA doubling time;
RECIST = Response Evaluation Criteria in Solid Tumors

### 6.3.7 Biomarker and Central Clinical Laboratory Analyses

The following laboratory analyses (Table 10. Summary of Sample Collection for Central Laboratory Analyses) are performed.

TABLE 10
Summary of Sample Collection for Central Laboratory Analyses

| Category | Sample Type | Type of Analyses |
|---|---|---|
| Viral | Saliva, feces, | Viral shedding is analyzed by |
| Shedding | blood, and | quantitative reverse transcription |
| | urine | PCR to quantify the copies of |
| | | nucleoprotein RNA |
| Viral | Serum, urine, | Viral Infectivity is analyzed |
| Infectivity | and saliva | by RCV assay to measure the |
| (RCV) | | number of replication competent |
| | | viral vectors |
| Biomarker | Blood/plasma | Exome and mutational changes |
| | | (ctDNA) |
| | | Transcriptional analysis (RNA-seq) |
| Biomarker | Serum | Cytokines |
| | | Neutralizing antibodies |
| | | bAb (anti-PAP, anti-PSA, anti- |
| | | PSMA antibodies) |
| Biomarker | Tumor tissue | IHC TIL |
| | | Transcriptome analysis (RNA-seq) |
| | | WES analysis |
| Immunogenicity | Blood | Intracellular cytokine staining |
| | | (ICS) panel CD4 and CD8: IFN-γ, |
| | | TNF-α, IL-2, CD107a, and CD154 |
| | | from PBMC samples |
| Immunogenicity | Blood | ELISpot assay measuring secreted |
| | | IFN-γ using PSA, PAP, and PSMA- |
| | | based peptides and LCMV NP peptides |
| | | from PBMC samples |

Abbreviations: bAb = binding antibody assay, CD4 = cluster of differentiation 4, CD8 = cluster of differentiation 8, ctDNA = circulating tumor deoxyribonucleic acid, ELISpot = enzyme-linked immune absorbent spot, ICS = intracellular cytokine staining, IFN-γ = interferon-gamma, IHC = immunohistochemistry, LCMV = lymphocytic choriomeningitis virus, NP = nucleoprotein, PAP = prostatic acid phosphatase, PBMC = peripheral blood mononuclear cell, PSA = prostate-specific antigen, PSMA = prostate-specific membrane antigen, RCV = replication-competent virus, RNA = ribonucleic acid, TIL = tumor-infiltrating lymphocyte, TNF α = tumor necrosis factor alpha, WES = whole exome sequencing.

### 6.4 Treating Metastatic Castration-Resistant Prostate Cancer Patients

### 6.4.1 Viral Vector-Based Therapeutic Immunotherapies

Two individual viral vector-based therapeutic immunotherapies, as described in Table 11. Descriptions of the Viral Vector-Based Therapeutic Immunotherapies below, are studied, exploring the following treatment regimen:
artPICV-PAP-NP/PSA-GP is administered first in alternating sequence with artLCMV-PAP-NP/PSA-GP.

TABLE 11
Descriptions of the Viral Vector-Based Therapeutic Immunotherapies

| | Vector | Antigen(s) | |
|---|---|---|---|
| IMP Name | backbone | expressed | Description |
| artLCMV- | LCMV | PAP & PSA | A genetically engineered LCMV |
| PAP-NP/ | | | vector based on the tri-segmented, |
| PSA-GP | | | replicating arenavirus vector |
| | | | technology, based on the LCMV |
| | | | strain Clone 13 with the viral |
| | | | surface glycoprotein from LCMV |
| | | | strain WE, expressing prostate |
| | | | cancer-related antigens PAP and PSA. |
| artPICV- | PICV | PAP & PSA | A genetically engineered PICV |
| PAP-NP/ | | | vector based on the tri-segmented, |
| PSA-GP | | | replicating arenavirus vector |
| | | | technology, based on the PICV |
| | | | strain p18, expressing PAP/PSA. |

Abbreviations:
LCMV = lymphocytic choriomeningitis virus,
PAP = prostatic acid phosphatase,
PSA = prostate-specific antigen,
PICV = pichinde virus,

### 6.4.2 Treatment Overview

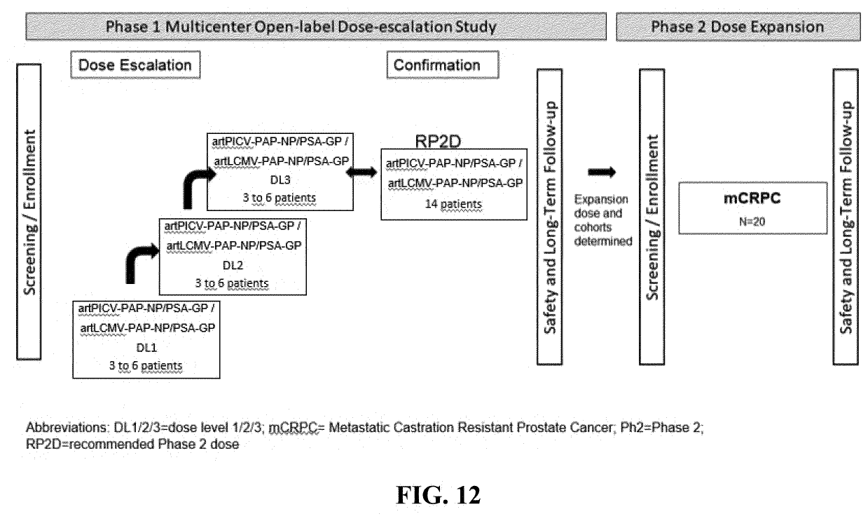

Adult patients with metastatic castration-resistant prostate cancer (mCRPC) are treated in two phases: a Phase 1 Dose Escalation and recommended phase 2 dose (RP2D) confirmation, and a Phase 2 Dose Expansion. Schematics of the study design are presented in FIG. 12.

### (i) Phase I Dose Escalation

The Phase I Dose Escalation evaluates artPICV-PAP-NP/PSA-GP/artLCMV-PAP-NP/PSA-GP alternating 2-vector therapy for safety and tolerability, preliminary efficacy, immunogenicity and determination of a safe recommended Phase 2 dose (RP2D). In the alternating 2-vector treatment artPICV-PAP-NP/PSA-GP is administered first in alternating sequence with artLCMV-PAP-NP/PSA-GP.

Study treatment is administered as indicated in Section 6.4.5(i).

### (ii) Phase II Dose Expansion

Phase II Dose Expansion commences upon completion of the Phase I Dose Escalation and assesses artPICV-PAP-NP/PSA-GP/artLCMV-PAP-NP/PSA-GP alternating 2-vector therapy at the RP2D defined in the Phase 1 part of the study.

Study treatment regimen will be based on the safety, efficacy, biomarker, and immunogenicity results from the Phase I Dose Escalation portion of the study as indicated in Section 6.4.5(ii).

### 6.4.3 Patient Population: Inclusion Criteria

Participants with histologically- or cytologically-confirmed adenocarcinoma of the prostate who have demonstrated castration condition with serum testosterone levels of <50 ng/dL (1.7 nmol/L) and at least 1 measurable soft tissue lesion and/or ≥1 detectable bone metastases will be enrolled in the study.

### For all Patients:

Patients who meet all of the following criteria are eligible to participate in the study:
1. Male patients ≥18 years of age at the time of signing the Informed Consent Form (ICF).
2. Willing and able to give voluntary informed consent for participation in the study.
3. Histologically or cytologically confirmed adenocarcinoma of the prostate without neuroendocrine differentiation or small cell features.
4. Documented castration condition with serum testosterone levels of <50 ng/dL (1.7 nmol/L). The castration condition can be obtained by bilateral orchiectomy or use of luteinizing hormone-releasing hormone (LHRH) analog (agonist or antagonist). Patients who have not undergone surgical bilateral orchiectomy must be willing to continue LHRH analog during the course of the study.
5. Patients must have ≥1 measurable soft tissue lesion and/or ≥1 detectable bone metastases. Soft tissue lesions can be assessed by computed tomography (CT) and/or magnetic resonance imaging (MRI) for tumor response following Response Evaluation Criteria in Solid Tumors (RECIST) v1.1 and immune RECIST (iRECIST) during study conduct.
6. Patients must have had assessed disease progression on standard of care therapy. Disease progression is defined by one or more of the following criteria according to Prostate Cancer Clinical Trials Working Group 3 (PCWG3) guidance:
   For patients who manifest disease progression solely as a rising PSA level, PCWG3 requires at least two consecutive rising PSA values with ≥3 week apart (not limited to the 28-day screening period) and a minimum starting value of 1.0 ng/mL. Most recent PSA level must be obtained within 21 days prior to first study drug treatment.
   For patients with measurable nodal or visceral lesions, disease progression of one of these lesions define by RECIST 1.1 is sufficient for eligibility independent of PSA. In case of lymph node ≥15 mm in diameter, it is considered measurable and used to evaluate change of size.
   For patients with bone metastases, progression is defined by the appearance of ≥2 new lesions by bone scan or other scans (e.g. MRI)
7. Antiandrogen withdrawal followed by progression must take place at least ≥4 weeks before enrollment unless case-specific exceptions are applied. LHRH agonists or antagonists should be continued.
8. Unless case-specific exceptions are applied, patients must have:
   An archival soft tissue tumor specimen collected following the patients' progression from last treatment or the ability to provide fresh soft tissue biopsy specimen prior to dosing. If archival sample is provided, the sample must not be older than 2 years.
   A soft tissue lesion potentially available for biopsy on-study.
9. Eastern Cooperative Oncology Group (ECOG) performance status (PS) of 0 to 1.
10. Prior curative radiation therapy must have been completed at least 4 weeks prior to study drug administration. Prior focal palliative radiotherapy must have been completed at least 2 weeks prior to study drug administration.
11. Screening laboratory values must meet the following criteria and should be obtained within 28 days prior to study treatment administration:
   Absolute neutrophil count≥1,500/mm³ (1.5×10⁹/L)
   Platelets ≥100×10³/mm³ (100×10⁹/L)
   Hemoglobin ≥9 g/dL (90 g/L) or ≥5.6 mmol/L
   Serum creatinine ≤1.5×upper limit of normal (ULN) or creatinine clearance >30 mL/min for patients with creatinine levels ≥1.5×institutional ULN (using the Cockcroft-Gault formula)
   Aspartate aminotransferase (AST)/alanine aminotransferase (ALT)≤2.5×ULN or ≤5×ULN for patients with liver metastases
   Total bilirubin ≤1.5×ULN or direct bilirubin ≤ULN for patients with total bilirubin levels >1.5×ULN
   Albumin≥3 g/dL
   International Normalized Ratio (INR) or Prothrombin Time (PT) 1.5×ULN (unless patient is receiving anticoagulant therapy as long as PT or Partial Thromboplastin Time (PTT) is within therapeutic range of intended use of anticoagulants)
   Activated Partial Thromboplastin Time (aPTT) or Partial Thromboplastin Time (PTT)≤1.5×ULN (unless patient is receiving anticoagulant therapy as long as PT or PTT is within therapeutic range of intended use of anticoagulants)

In some embodiments, inclusion criteria may include that patients have been treated with at least 1 targeted endocrine therapy (defined as second generation antiandrogen therapies that include but are not limited to abiraterone acetate with prednisone, enzalutamide, and next generation targeted agents such as ARN-509), and/or at least 1 regimen/line of chemotherapy that contained docetaxel, and/or no prior chemotherapy regimens, and/or no more than 3 regimens/lines of the aforementioned treatments (having failed/progressed on prior therapy).

In some embodiments, inclusion criteria may include that patients have had assessed disease progression on standard of care therapy, and for patients who manifest disease progression solely as a rising PSA level, PCWG3 requires at least two consecutive rising PSA values with ≥3 week apart (not limited to the 28-day screening period) and a minimum starting value of 1.0 ng/mL, for patients receiving flutamide, at least one of the PSA values must be obtained ≥4 weeks after flutamide discontinuation, for patients receiving bicalutamide or nilutamide, at least one of the PSA values must be obtained ≥6 week after antiandrogen discontinuation.

### 6.4.4 Dose Escalation Guidelines—Provisional Dose Levels Explored

artLCMV-PAP-NP/PSA-GP and artPICV-PAP-NP/PSA-GP are administered IV. A starting dose of 1×10⁶ RCV FFU per dose per patient for each vector is administered. Furthermore, a dose escalation plan for the Phase 1 portion of the clinical study permits dose increments of up to one log order between cohorts (i.e., 10⁶, 10⁷, 10⁸ RCV FFU).

For Phase 1 Dose Escalation, the proposed human starting dose of artLCMV-PAP-NP/PSA-GP and artPICV-PAP-NP/PSA-GP is 1×10⁶ RCV FFU. A non-limiting example of potential dose escalation is given in Table 12. Provisional Dose Level for Alternating 2-Vector Treatment of artPICV-PAP-NP/PSA-GP and artLCMV-PAP-NP/PSA-GP

TABLE 12
Provisional Dose Level for Alternating 2-Vector Treatment
of artPICV-PAP-NP/PSA-GP and artLCMV-PAP-NP/PSA-GP

| | artPICV-PAP-NP/PSA-GP | artLCMV-PAP-NP/PSA-GP |
|---|---|---|
| Level | Provisional Dose | Provisional Dose |
| 1 | 1 × 10⁶ RCV FFU | 1 × 10⁶ RCV FFU |
| (starting | | |
| dose) | | |
| 2 | 1 × 10⁷ RCV FFU | 1 × 10⁷ RCV FFU |
| 3 | 1 × 10⁸ RCV FFU | 1 × 10⁸ RCV FFU |

Abbreviations:
FFU = focus-forming units,
RCV = replication-competent virus

### 6.4.5 Treatment Regimen and Cycle Duration

Patients are treated until they experience unacceptable treatment-related adverse events, disease progression (confirmed by immune Response Evaluation Criteria in Solid Tumors (iRECIST) for visceral/soft tissue metastasis or Prostate Cancer Work 3 (PCW3) for bone metastasis) or withdraw consent.

### (i) Phase I Dose Escalation

artPICV-PAP-NP/PSA-GP and artLCMV-PAP-NP/PSA-GP are given in alternating IV administrations. artPICV-PAP-NP/PSA-GP is administered first, followed by artLCMV-PAP-NP/PSA-GP.

For Cycles 1 and 2, a treatment cycle is defined as a period of 42 days. artPICV-PAP-NP/PSA-GP is administered first, followed by artLCMV-PAP-NP/PSA-GP, alternating treatment every three weeks (21 days) for the first four administrations.
artPICV-PAP-NP/PSA-GP is administered IV on Day 1 of Cycles 1 and 2.
artLCMV-PAP-NP/PSA-GP is administered IV on Day 22 of Cycles 1 and 2.

For Cycle 3 and subsequent cycles, a treatment cycle is defined as a period of 84 days. Cycle 3 Day 1 starts following the completion of Cycle 2 Day 42. artPICV-PAP-NP/PSA-GP and artLCMV-PAP-NP/PSA-GP dose administrations in Cycle 3 and subsequent cycles have a time window of ±7 days. artPICV-PAP-NP/PSA-GP and artLCMV-PAP-NP/PSA-GP doses alternate every six weeks (42 days) as follows:
artPICV-PAP-NP/PSA-GP is administered IV on Day 1 of Cycle 3 and subsequent cycles.
artLCMV-PAP-NP/PSA-GP is administered IV on Day 43 of Cycle 3 and subsequent cycles.

In one embodiment, the first 5 doses may be administered in 3 weeks intervals, from the sixth dose onwards, doses may be administered in 6 weeks intervals.

For Phase I Dose Escalation, administration schedules for artPICV-PAP-NP/PSA-GP and artLCMV-PAP-NP/PSA-GP regimen may be modified for the next cohort based on safety, efficacy, or biomarker data.

For Phase I Dose Escalation, the following information is collected: (1) Incidence of DLTs from the first study drug administered during the DLT observation period; (2) Safety: type, frequency, and severity of AEs and SAEs; (3) Tolerability: dose interruptions, reductions and dose intensity, and evaluations of laboratory values; (4) overall survival (OS); (5) Progression-free survival (PFS) using RECIST v1.1/iRECIST (soft tissues) and PCWG3 (bone) criteria; (6) Overall response rate (ORR), disease control rate (DCR), and duration of response (DOR) using RECIST v1.1/iRECIST (soft tissues) and PCWG3 (bone) criteria; (7) Proportion of patients with PSA response (≥50% decrease in PSA from baseline to the lowest postbaseline PSA result, confirmed by a second consecutive PSA assessment at least 3 weeks later); (8) Best PSA response at any time; (9) Time to PSA Progression (PSA PFS); (10) Measurement of prostate cancer-associated antigen (PAP and PSA)-specific T cell responses; (11) Characterization of prostate cancer-associated antigen (PAP and PSA)-specific T cells; and (12) Assessment of biomarkers in tumor specimens and circulating tumor cells in blood and plasma.

### (ii) Phase II Dose Expansion

Treatment regimen and cycle duration of the arenaviral vectors are the same as described in Phase I.

In some treatment groups patients will receive a combination therapy of a medication that is approved for treatment of advanced prostate cancer together with artLCMV-PAP-NP/PSA-GP and artPICV-PAP-NP/PSA-GP.

For Phase II Dose Expansion, the following information is collected: (1) Proportion of patients with PSA response (i.e., ≥50% decrease in PSA from baseline to the lowest post-baseline PSA result, confirmed by a second consecutive PSA assessment at least 3 weeks later); (2) Tumor response using RECIST v.11/iRECIST (soft tissue), PCWG3 (bone) criteria; (3) overall survival (OS); (4) Progression-free survival (PFS) using RECIST v1.1/iRECIST (soft tissues) and PCWG3 (bone) criteria; (5) Overall response rate (ORR), disease control rate (DCR), and duration of response (DOR) using RECIST v.11/iRECIST (soft tissues) and PCWG3 (bone) criteria; (6) Best PSA response at any time; (7) Time to PSA Progression (PSA PFS); (8) Safety: type, frequency, and severity of AEs and SAEs; (8) Tolerability: dose interruptions, reductions and dose intensity, and evaluations of laboratory values; (9) Measurement of prostate cancer-associated antigen (PAP and PSA)-specific T cell responses; (10) Characterization of prostate cancer-associated antigen (PAP and PSA)-specific T cells; (11) Assessment of biomarkers in tumor specimens and circulating tumor cells in blood and plasma.

### 6.4.6 Efficacy Variables

Efficacy is assessed using PSA response according to PCWG3 (Scher et al. 2016), bone response according to PCWG3, and soft tissue response according to RECIST v1.1 and iRECIST (secondary efficacy endpoints).

PSA levels are assessed at baseline and every 3 weeks starting at Cycle 1 Day 1. Tumor and bone scans are performed every 9 weeks (±7 days) in the first year after Day 1 of Cycle 1 until objective radiological disease progression.

The imaging modalities used for RECIST assessment are CT or MRI scans of the chest, abdomen and pelvis. Any other areas of disease involvement are additionally investigated based on the signs and symptoms of individual patients.

Bone lesions are assessed by bone scintigraphy commonly performed with Technetium-99 (bone scans). Bone lesions are assessed by bone scan and are not part of the RECIST v.1.1 malignant soft tissue assessment. Positive hot spots on the bone scan are considered significant and unequivocal sites of malignant disease are recorded as metastatic bone lesions.

Table 13. PCWG3 Criteria of Progression by Disease Manifestation shows criteria used in Phase I and Phase II to assess efficacy and progression on the basis of changes in PSA, bone metastases, and measurable disease.

TABLE 13
PCWG3 Criteria of Progression by Disease Manifestation

| | Evaluation Criteria |
|---|---|
| Assessment | for Disease Progression |
| PSA | Obtain sequence of rising values |
| | at a minimum of 1-week intervals |
| | 1.0 ng/mL minimal starting value |
| | if confirmed rise is only |
| | indication of progression unless |
| | pure small-cell carcinoma |
| | Estimate pre-therapy PSA-DT if ≥3 |
| | values available ≥4 weeks apart |
| Target | Nodal or visceral progression |
| lesions | sufficient for trial entry |
| | independent of PSA |
| | Measurable lesions not required for |
| | entry |
| | Use RECIST v1.1 to record soft-tissue |
| | (nodal and visceral) lesions as target |
| | or nontarget |
| | Previously normal (<1.0 cm) lymph |
| | nodes must have grown by ≥5 mm in |
| | the short axis from baseline or nadir |
| | and be ≥1.0 cm in the short axis to be |
| | considered to have progressed |
| | If the node progresses to ≥1.5 cm in |
| | the short axis, it is measurable; nodes |
| | that have progressed to 1.0 to <1.5 cm |
| | are pathologic, subject to clinical |
| | discretion, and non-measurable |
| | For existing pathologic adenopathy, |
| | progression is defined per RECIST v1.1 |
| | Record presence of nodal and/or visceral |
| | disease separately: |
| | Nodal sites: Locoregional: pelvic only; |
| | Extrapelvic: retroperitoneal, mediastinal, |
| | thoracic, or other |
| | Visceral sites: lung, liver, adrenal, CNS |
| Prostate | Record prior treatment of primary tumor |
| | Perform directed pelvic imaging (CT, MRI, |
| | PET/CT, endorectal MRI, transrectal |
| | ultrasound) to document presence or absence |
| | of disease |
| Bone | Progression = appearance of ≥2 or more |
| | new lesions |
| | Confirm ambiguous results by other imaging |
| | modalities (e.g., CT or MRI), but only |
| | positivity on the bone scan defines |
| | metastatic disease to bone |

Abbreviations:
CNS = central nervous system;
PSA = prostate-specific antigen;
CT = computed tomography;
MRI = magnetic resonance imaging;
PCWG3 = Prostate Cancer Clinical Trials Working Group 3;
PET = positron emission tomography;
PSA-DT = PSA doubling time;
RECIST = Response Evaluation Criteria in Solid Tumors

### 6.4.7 Exploratory Biomarker, T Cell Responses, PD Biomarkers, and Other Central Clinical Laboratory Analyses

The following laboratory analyses are performed.

### Viral Shedding

Samples from saliva, blood, and urine are collected from patients for viral shedding analysis. Viral shedding will be analyzed by quantitative reverse transcription PCR to quantify the copies of nucleoprotein RNA and may be coupled with infectivity assay to characterize the shed material to confirm absence of infectious virus.

### T Cell Immune Responses

Cellular immune responses are measured by enzyme-linked immunosorbent spot (ELISpot) assay to assess secreted IFN-γ specific cells in peripheral blood mononuclear cells as an antigen specific immune response and CD8+ T cells functionality and antigen recognition by measuring IFN-γ, TNF-α, IL-2, CD107a via intracellular staining against artPICV-PAP-NP/PSA-GP and/or artLCMV-PAP-NP/PSA-GP (Table 14).

TABLE 14
Summary of Immunogenicity Analysis

| Sample | |
|---|---|
| Type | Immunogenicity Analysis |
| Blood | ICS panel CD4+ and CD8+: IFN-γ, |
| (PBMCs) | TNF-α, IL-2, CD107a and CD40L |
| | ELISpot assay measuring secreted |
| | IFN-γ using PSA and PAP based |
| | peptides, LCMV, and PICV NP peptides. |

CCD4 = cluster of differentiation 4;
CD8 = cluster of differentiation 8;
PSA = Prostate Specific Antigen and E6
PAP = Prostatic Acid Phosphatase;
ELISpot = enzyme-linked immune absorbent spot;
ICS = intracellular cytokine staining;
IFN-γ = interferon-gamma;
IL-2 = interleukin-2;
LMCV = lymphocytic choriomeningitis virus;
NP = nucleoprotein;
PBMC = peripheral blood mononuclear cell;
PICV = Pichinde Virus;
TNF-α = tumor necrosis factor alpha

### Pharmacodynamic Biomarkers

Additional biomarker research to identify factors important for artPICV-PAP-NP/PSA-GP and/or artLCMV-PAP-NP/PSA-GP therapy can be pursued to further investigate and understand determinants of response and/or resistance to therapy as well as determinants of adverse events during the clinical trial. Biospecimens, (tumor material and blood components; serum, plasma and PBMC) may be used for immunostaining of tissue markers, genomic, and transcriptional analyses.

Assays may include but are not limited to:
Germline (blood) Genetic Analyses (e.g., whole exome sequencing (WES), gene expression profile, RNA-sequencing):
Gene and Transcriptional Analyses in Tumor: ImmunoID NeXT platform will evaluate the presence specific T cell clones, mutational changes (TMB), Microsatellite Instability (MSI) gene signatures and identify somatic mutations in BRCA1/2 and other homologous recombination repair (HRR)-related genes and detect the presence of genomic scars indicative of Homologous recombination deficiency (HRD), which can be critical for clinical response to artPICV-PAP-NP/PSA-GP and/or artLCMV-PAP-NP/PSA-GP therapy as a single agent or in combination with other treatments.
Multiplex Immunofluorescent Immunohistochemistry (mIF): Tumor samples will be assessed for PD-L1/PD-1 expression, tumor infiltrating lymphocytes (TILs), exhaustion markers and CD103+ tumor-infiltrating lymphocytes expression which has been shown in many studies to be correlated to overall better survival in many different cancers.
Circulating Tumor Cells (CTC) and Circulating Tumor DNA (ctDNA): Enumeration of CTC has shown to be a biomarker of prognosis and response in metastatic castration-resistant prostate cancer (mCRPC). Decreased levels of CTC count is associated with improved progression free survival (PFS) and overall survival (OS). (Cristofanilli, 2004, Hayes, 2006) Blood sample will be collected at baseline and during the treatment to measure CTCs, which have been shown to be correlated to prognosis and response in metastatic castration-resistant prostate cancer (mCRPC). Additionally, plasma/serum will be collected at pre-defined timepoints in addition to tumor tissue, to investigate the genomic landscape and characterize tumor-associated copy number alterations (CNAs), single-nucleotide variations (SNVs), and commonly observed rearrangements in patients.
Serum biomarkers analysis: Humoral immunity (anti-PSA/PAP antibodies and anti-vector neutralizing antibodies) will also be explored by and enzyme-linked immunosorbent assay (ELISA) and LCMV and PICV neutralizing antibody will be analyzed with neutralizing assay. Cytokines and chemokines will be studied by using Meso Scale Discovery (MSD) at predefined timepoints to study cytokine secretion profiles.

### 7. EQUIVALENTS

The S segments, genome segments, viral particles, nucleic acids, methods, host cells, compositions, and kits disclosed herein are not to be limited in scope by the specific embodiments described herein. Indeed, various modifications of S segments, genome segments, viral particles, nucleic acids, methods, host cells, compositions, and kits in addition to those described become apparent to those skilled in the art from the foregoing description and accompanying figures. Such modifications are intended to fall within the scope of the appended claims.

Various publications, patents and patent applications are cited herein, the disclosures of which are incorporated b reference in their entireties.

8. SEQUENCE LISTING

| SEQ | | |
|---|---|---|
| ID | | |
| NO. | Description | Sequence |
| 1 | Amino acid | MRAAPLLLARAASLSLGFLFLLFFWLDRSVLAKELKFVTL |
| | sequence of PAP | VFRHGDRSPIDTFPTDPIKESSWPQGFGQLTQLGMEQHYEL |
| | | GEYIRKRYRKFLNESYKHEQVYIRSTDVDRTLMSAMTNLA |
| | | ALFPPEGVSIWNPILLWQPIPVHTVPLSEDQLLYLPFRNCPR |
| | | FQELESETLKSEEFQKRLHPYKDFIATLGKLSGLHGQDLFGI |
| | | WSKVYDPLYCESVHNFTLPSWATEDTMTKLRELSELSLLS |
| | | LYGIHKQKEKSRLQGGVLVNEILNHMKRATQIPSYKKLIM |
| | | YSAHDTTVSGLQMALDVYNGLLPPYASCHLTELYFEKGE |
| | | YFVEMYYRNETQHEPYPLMLPGCSPSCPLERFAELVGPVIP |
| | | QDWSTECMTTNSHQGTEDSTD |
| 2 | Amino acid | MWVPVVFLTLSVTWIGAAPLILSRIVGGWECEKHSQPWQV |
| | sequence of PSA | LVASRGRAVCGGVLVHPQWVLTAAHCIRNKSVILLGRHSL |
| | | FHPEDTGQVFQVSHSFPHPLYDMSLLKNRFLRPGDDSSHD |
| | | LMLLRLSEPAELTDAVKVMDLPTQEPALGTTCYASGWGSI |
| | | EPEEFLTPKKLQCVDLHVISNDVCAQVHPQKVTKFMLCAG |
| | | RWTGGKSTCSGDSGGPLVCNGVLQGITSWGSEPCALPERP |
| | | SLYTKVVHYRKWIKDTIVANP |
| 3 | Amino acid | MWNLLHETDSAVATARRPRWLCAGALVLAGGFFLLGFLF |
| | sequence of | GWFIKSSNEATNITPKHNMKAFLDELKAENIKKFLHNFTQI |
| | “PSMA1” | PHLAGTEQNFQLAKQIQSQWKEFGLDSVELAHYDVLLSYP |
| | | NKTHPNYISIINEDGNEIFNTSLFEPPPPGYENVSDIVPPFSAF |
| | | SPQGMPEGDLVYVNYARTEDFFKLERDMKINCSGKIVIAR |
| | | YGKVFRGNKVKNAQLAGAKGVILYSDPADYFAPGVKSYP |
| | | DGWNLPGGGVQRGNILNLNGAGDPLTPGYPANEYAYRRG |
| | | IAEAVGLPSIPVHPIGYYDAQKLLEKMGGSAPPDSSWRGSL |
| | | KVPYNVGPGFTGNFSTQKVK |
| 4 | Amino acid | MHIHSTNEVTRIYNVIGTLRGAVEPDRYVILGGHRDSWVF |
| | sequence of | GGIDPQSGAAVVHEIVRSFGTLKKEGWRPRRTILFASWDA |
| | “PSMA2” | EEFGLLGSTEWAEENSRLLQERGVAYINADSSIEGNYTLRV |
| | | DCTPLMYSLVHNLTKELKSPDEGFEGKSLYESWTKKSPSP |
| | | EFSGMPRISKLGSGNDFEVFFQRLGIASGRARYTKNWETN |
| | | KFSGYPLYHSVYETYELVEKFYDPMFKYHLTVAQVRGGM |
| | | VFELANSIVLPFDCRDYAVVLRKYADKIYSISMKHPQEMK |
| | | TYSVSFDSLFSAVKNFTEIASKFSERLQDFDKSNPIVLRMM |
| | | NDQLMFLERAFIDPLGLPDRPFYRHVIYAPSSHNKYAGESF |
| | | PGIYDALFDIESKVDPSKAWGEVKRQIYVAAFTVQAAAET |
| | | LSEVA |
| 5 | Nucleotide | ATGAGAGCTGCACCCCTTCTCCTGGCCAGGGCAGCAAG |
| | sequence of PAP | CCTCAGCCTTGGCTTCTTGTTTCTGCTTTTTTTCTGGCTG |
| | | GACAGAAGTGTGCTTGCCAAGGAGTTGAAGTTTGTGAC |
| | | TTTGGTGTTCAGGCATGGAGACAGAAGTCCCATTGACAC |
| | | CTTTCCCACAGACCCCATCAAGGAATCCTCATGGCCTCA |
| | | AGGATTTGGTCAACTCACTCAACTGGGCATGGAGCAGC |
| | | ACTATGAACTTGGGGAGTACATCAGAAAGAGATACAGA |
| | | AAATTCTTGAATGAGTCCTACAAACATGAACAGGTTTAC |
| | | ATCAGAAGCACAGATGTTGACAGGACTTTGATGAGTGC |
| | | CATGACAAACCTGGCAGCCCTGTTCCCCCCTGAAGGTGT |
| | | CAGCATCTGGAATCCCATTCTCCTTTGGCAACCCATCCC |
| | | AGTGCACACAGTTCCTCTTTCTGAAGATCAGTTGCTCTA |
| | | CCTGCCTTTCAGGAATTGTCCAAGGTTTCAAGAACTTGA |
| | | GAGTGAAACTTTGAAATCAGAAGAATTTCAGAAGAGGC |
| | | TGCACCCTTACAAGGATTTCATAGCCACCTTGGGAAAGC |
| | | TTTCAGGGTTGCATGGGCAAGACCTTTTTGGCATTTGGA |
| | | GCAAAGTCTATGACCCTTTATATTGTGAGAGTGTTCACA |
| | | ATTTCACCTTGCCTTCTTGGGCCACTGAGGACACCATGA |
| | | CAAAGTTGAGAGAATTGTCAGAATTGTCCCTCCTGTCTC |
| | | TCTATGGCATTCACAAGCAGAAAGAGAAATCCAGGCTC |
| | | CAAGGGGGTGTCCTGGTCAATGAAATCCTGAATCACAT |
| | | GAAGAGAGCAACTCAGATCCCAAGCTACAAAAAACTCA |
| | | TCATGTATTCTGCTCATGACACAACTGTGAGTGGCCTGC |
| | | AGATGGCTCTAGATGTTTACAATGGCCTCCTCCCTCCCT |
| | | ATGCTTCTTGCCACTTGACAGAATTGTATTTTGAGAAGG |
| | | GGGAGTACTTTGTGGAGATGTACTACAGGAATGAGACC |
| | | CAGCATGAGCCTTATCCTCTCATGCTGCCTGGCTGCAGC |
| | | CCCAGTTGTCCTCTTGAGAGATTTGCTGAGCTGGTTGGC |
| | | CCTGTGATCCCTCAGGACTGGTCAACTGAGTGCATGACA |
| | | ACAAACAGTCATCAAGGAACTGAGGACAGCACAGATTA |
| | | G |
| 6 | Nucleotide | ATGTGGGTCCCTGTGGTCTTCCTCACCCTGTCTGTGACTT |
| | sequence of PSA | GGATTGGAGCTGCCCCCCTCATCCTGTCCAGGATTGTGG |
| | | GTGGCTGGGAGTGTGAGAAGCATTCCCAACCCTGGCAG |
| | | GTGCTGGTGGCCTCCAGAGGCAGGGCTGTGTGTGGGGG |
| | | GGTCCTGGTGCACCCCCAGTGGGTCCTCACTGCTGCCCA |
| | | CTGCATCAGGAACAAGAGTGTGATCTTGCTGGGGAGGC |
| | | ACAGCCTGTTCCATCCTGAAGACACAGGCCAGGTCTTCC |
| | | AGGTCAGCCACAGCTTCCCCCACCCCCTCTATGACATGA |
| | | GCCTCCTGAAGAACAGATTCCTCAGGCCTGGTGATGACT |
| | | CCAGCCATGACCTCATGCTGCTCAGGCTGTCAGAGCCTG |
| | | CAGAGCTCACTGATGCTGTGAAGGTCATGGACCTGCCC |
| | | ACCCAGGAGCCAGCCCTGGGGACCACCTGCTATGCCTC |
| | | AGGCTGGGGCAGCATTGAACCAGAGGAGTTCTTGACCC |
| | | CCAAGAAACTTCAGTGTGTGGACCTCCATGTCATCTCCA |
| | | ATGATGTGTGTGCCCAAGTTCACCCCCAGAAGGTCACCA |
| | | AGTTCATGCTGTGTGCTGGAAGATGGACAGGGGGCAAA |
| | | AGCACCTGCTCTGGTGACTCTGGGGGCCCCCTTGTGTGC |
| | | AATGGTGTGCTCCAAGGCATCACCTCCTGGGGCAGTGA |
| | | GCCATGTGCCCTGCCTGAAAGGCCTTCCCTGTACACCAA |
| | | GGTGGTTCATTACAGGAAGTGGATCAAGGACACAATTG |
| | | TGGCCAACCCCTGA |
| 7 | Nucleotide | ATGTGGAATCTTCTTCATGAAACTGACTCAGCTGTGGCC |
| | sequence of | ACAGCCAGAAGACCCAGGTGGCTGTGTGCAGGGGCCCT |
| | “PSMA1” | TGTTCTTGCAGGTGGTTTTTTTCTCCTTGGCTTCCTCTTT |
| | | GGTTGGTTCATCAAGTCTTCAAATGAAGCAACCAACATC |
| | | ACTCCAAAGCACAACATGAAAGCATTTTTGGATGAATT |
| | | GAAAGCTGAGAACATCAAGAAGTTTTTGCACAATTTCA |
| | | CACAGATTCCACATTTGGCAGGAACAGAACAAAACTTT |
| | | CAGCTTGCAAAGCAAATTCAATCCCAGTGGAAAGAATT |
| | | TGGTCTGGATTCTGTTGAGCTGGCTCATTATGATGTTCT |
| | | GTTGTCCTACCCAAACAAGACTCATCCCAACTACATCTC |
| | | AATCATCAATGAAGATGGAAATGAGATTTTCAACACCT |
| | | CTTTGTTTGAACCACCACCTCCAGGATATGAAAATGTGT |
| | | CAGACATTGTTCCTCCTTTCAGTGCTTTTTCTCCTCAAGG |
| | | CATGCCAGAGGGAGATCTGGTCTATGTCAACTATGCAA |
| | | GAACTGAAGACTTTTTCAAATTGGAAAGAGACATGAAA |
| | | ATCAATTGCTCTGGGAAAATTGTCATTGCCAGATATGGG |
| | | AAAGTTTTCAGAGGCAACAAGGTGAAAAATGCCCAGCT |
| | | GGCAGGTGCCAAAGGAGTCATTCTCTACTCTGACCCTGC |
| | | AGACTATTTTGCTCCTGGGGTGAAATCTTATCCTGATGG |
| | | TTGGAATCTTCCTGGAGGTGGTGTCCAGAGGGGCAACA |
| | | TCCTCAATCTGAATGGTGCAGGAGATCCACTCACCCCAG |
| | | GTTACCCAGCAAATGAATATGCTTACAGAAGAGGAATT |
| | | GCAGAGGCTGTTGGTCTTCCCAGCATTCCTGTTCATCCA |
| | | ATTGGATACTATGATGCCCAGAAACTCCTGGAAAAGAT |
| | | GGGTGGTTCAGCACCCCCAGACAGCAGCTGGAGAGGCA |
| | | GTCTCAAAGTGCCATACAATGTTGGCCCTGGTTTCACAG |
| | | GAAACTTTTCCACTCAAAAAGTCAAATGA |
| 8 | Nucleotide | ATGCACATCCATTCAACCAATGAAGTGACAAGAATTTA |
| | sequence of | CAATGTGATTGGAACTCTCAGAGGAGCAGTGGAACCAG |
| | “PSMA2” | ACAGATATGTCATTCTGGGAGGTCACAGGGACTCCTGG |
| | | GTGTTTGGTGGAATTGACCCTCAGAGTGGAGCAGCTGTG |
| | | GTTCATGAAATTGTCAGGAGTTTTGGAACACTGAAAAA |
| | | GGAAGGGTGGAGACCCAGAAGAACAATTTTGTTTGCAA |
| | | GCTGGGATGCAGAAGAATTTGGTCTTCTTGGTTCAACTG |
| | | AGTGGGCAGAGGAGAACTCAAGACTCCTTCAGGAGAGA |
| | | GGAGTAGCTTACATCAATGCTGACTCATCTATTGAAGGA |
| | | AATTACACTCTGAGAGTTGATTGCACTCCACTAATGTAC |
| | | AGCTTGGTTCACAATCTGACAAAAGAGCTGAAAAGCCC |
| | | TGATGAAGGGTTTGAAGGAAAATCTCTTTATGAAAGTTG |
| | | GACAAAAAAAAGTCCCTCCCCAGAGTTCAGTGGAATGC |
| | | CCAGGATCAGCAAATTGGGATCTGGAAATGATTTTGAG |
| | | GTGTTCTTCCAAAGACTTGGAATTGCTTCAGGCAGAGCA |
| | | AGGTACACCAAGAATTGGGAAACCAACAAATTCAGTGG |
| | | TTATCCACTATATCACAGTGTTTATGAAACATATGAGTT |
| | | GGTGGAAAAGTTTTATGATCCAATGTTCAAATATCATCT |
| | | GACTGTGGCACAGGTCAGAGGAGGGATGGTGTTTGAGC |
| | | TGGCCAATTCCATAGTTCTCCCTTTTGATTGCAGAGATT |
| | | ATGCTGTGGTTTTGAGAAAGTATGCTGACAAAATTTACA |
| | | GCATTTCAATGAAACATCCACAGGAAATGAAGACATAC |
| | | AGTGTCTCATTTGATTCACTTTTTTCTGCAGTGAAGAATT |
| | | TCACAGAAATTGCTTCCAAGTTCAGTGAAAGGCTTCAGG |
| | | ACTTTGACAAAAGCAACCCAATTGTTTTGAGAATGATGA |
| | | ATGATCAACTCATGTTTCTGGAAAGAGCATTCATTGATC |
| | | CCTTGGGGTTGCCAGACAGGCCTTTTTACAGGCATGTCA |
| | | TCTATGCCCCAAGCAGTCACAACAAGTATGCAGGGGAG |
| | | TCATTTCCAGGAATTTATGATGCTCTGTTTGACATTGAA |
| | | AGCAAAGTGGACCCTTCCAAGGCCTGGGGAGAAGTGAA |
| | | GAGACAGATTTATGTTGCAGCCTTCACAGTTCAGGCAGC |
| | | TGCAGAGACTTTGAGTGAAGTTGCTTAA |
| 9 | Nucleotide | ATGTGGAATCTTCTTCATGAAACTGACTCAGCTGTGGCC |
| | sequence of full | ACAGCCAGAAGACCCAGGTGGCTGTGTGCAGGGGCCCT |
| | PSMA | TGTTCTTGCAGGTGGTTTTTTTCTCCTTGGCTTCCTCTTT |
| | | GGTTGGTTCATCAAGTCTTCAAATGAAGCAACCAACATC |
| | | ACTCCAAAGCACAACATGAAAGCATTTTTGGATGAATT |
| | | GAAAGCTGAGAACATCAAGAAGTTTTTGCACAATTTCA |
| | | CACAGATTCCACATTTGGCAGGAACAGAACAAAACTTT |
| | | CAGCTTGCAAAGCAAATTCAATCCCAGTGGAAAGAATT |
| | | TGGTCTGGATTCTGTTGAGCTGGCTCATTATGATGTTCT |
| | | GTTGTCCTACCCAAACAAGACTCATCCCAACTACATCTC |
| | | AATCATCAATGAAGATGGAAATGAGATTTTCAACACCT |
| | | CTTTGTTTGAACCACCACCTCCAGGATATGAAAATGTGT |
| | | CAGACATTGTTCCTCCTTTCAGTGCTTTTTCTCCTCAAGG |
| | | CATGCCAGAGGGAGATCTGGTCTATGTCAACTATGCAA |
| | | GAACTGAAGACTTTTTCAAATTGGAAAGAGACATGAAA |
| | | ATCAATTGCTCTGGGAAAATTGTCATTGCCAGATATGGG |
| | | AAAGTTTTCAGAGGCAACAAGGTGAAAAATGCCCAGCT |
| | | GGCAGGTGCCAAAGGAGTCATTCTCTACTCTGACCCTGC |
| | | AGACTATTTTGCTCCTGGGGTGAAATCTTATCCTGATGG |
| | | TTGGAATCTTCCTGGAGGTGGTGTCCAGAGGGGCAACA |
| | | TCCTCAATCTGAATGGTGCAGGAGATCCACTCACCCCAG |
| | | GTTACCCAGCAAATGAATATGCTTACAGAAGAGGAATT |
| | | GCAGAGGCTGTTGGTCTTCCCAGCATTCCTGTTCATCCA |
| | | ATTGGATACTATGATGCCCAGAAACTCCTGGAAAAGAT |
| | | GGGTGGTTCAGCACCCCCAGACAGCAGCTGGAGAGGCA |
| | | GTCTCAAAGTGCCATACAATGTTGGCCCTGGTTTCACAG |
| | | GAAACTTTTCCACTCAAAAAGTCAAAATGCACATCCATT |
| | | CAACCAATGAAGTGACAAGAATTTACAATGTGATTGGA |
| | | ACTCTCAGAGGAGCAGTGGAACCAGACAGATATGTCAT |
| | | TCTGGGAGGTCACAGGGACTCCTGGGTGTTTGGTGGAAT |
| | | TGACCCTCAGAGTGGAGCAGCTGTGGTTCATGAAATTGT |
| | | CAGGAGTTTTGGAACACTGAAAAAGGAAGGGTGGAGAC |
| | | CCAGAAGAACAATTTTGTTTGCAAGCTGGGATGCAGAA |
| | | GAATTTGGTCTTCTTGGTTCAACTGAGTGGGCAGAGGAG |
| | | AACTCAAGACTCCTTCAGGAGAGAGGAGTAGCTTACAT |
| | | CAATGCTGACTCATCTATTGAAGGAAATTACACTCTGAG |
| | | AGTTGATTGCACTCCACTAATGTACAGCTTGGTTCACAA |
| | | TCTGACAAAAGAGCTGAAAAGCCCTGATGAAGGGTTTG |
| | | AAGGAAAATCTCTTTATGAAAGTTGGACAAAAAAAAGT |
| | | CCCTCCCCAGAGTTCAGTGGAATGCCCAGGATCAGCAA |
| | | ATTGGGATCTGGAAATGATTTTGAGGTGTTCTTCCAAAG |
| | | ACTTGGAATTGCTTCAGGCAGAGCAAGGTACACCAAGA |
| | | ATTGGGAAACCAACAAATTCAGTGGTTATCCACTATATC |
| | | ACAGTGTTTATGAAACATATGAGTTGGTGGAAAAGTTTT |
| | | ATGATCCAATGTTCAAATATCATCTGACTGTGGCACAGG |
| | | TCAGAGGAGGGATGGTGTTTGAGCTGGCCAATTCCATA |
| | | GTTCTCCCTTTTGATTGCAGAGATTATGCTGTGGTTTTGA |
| | | GAAAGTATGCTGACAAAATTTACAGCATTTCAATGAAA |
| | | CATCCACAGGAAATGAAGACATACAGTGTCTCATTTGAT |
| | | TCACTTTTTTCTGCAGTGAAGAATTTCACAGAAATTGCT |
| | | TCCAAGTTCAGTGAAAGGCTTCAGGACTTTGACAAAAG |
| | | CAACCCAATTGTTTTGAGAATGATGAATGATCAACTCAT |
| | | GTTTCTGGAAAGAGCATTCATTGATCCCTTGGGGTTGCC |
| | | AGACAGGCCTTTTTACAGGCATGTCATCTATGCCCCAAG |
| | | CAGTCACAACAAGTATGCAGGGGAGTCATTTCCAGGAA |
| | | TTTATGATGCTCTGTTTGACATTGAAAGCAAAGTGGACC |
| | | CTTCCAAGGCCTGGGGAGAAGTGAAGAGACAGATTTAT |
| | | GTTGCAGCCTTCACAGTTCAGGCAGCTGCAGAGACTTTG |
| | | AGTGAAGTTGCTTAA |
| 10 | PAP-NP-S- | gcgcaccggggatcCTAGGCTTTTTGGATTGCGCTTTCCTCTAG |
| | segment of | ATCAACTGGGTGTCAGGCCCTATCCTACAGAAGGATGA |
| | PMVS (12), | GAGCTGCACCCCTTCTCCTGGCCAGGGCAGCAAGCCTCA |
| | which is a PMVS | GCCTTGGCTTCTTGTTTCTGCTTTTTTTCTGGCTGGACAG |
| | of artLCMV- | AAGTGTGCTTGCCAAGGAGTTGAAGTTTGTGACTTTGGT |
| | PAP-NP/PSA-GP | GTTCAGGCATGGAGACAGAAGTCCCATTGACACCTTTCC |
| | | CACAGACCCCATCAAGGAATCCTCATGGCCTCAAGGAT |
| | | TTGGTCAACTCACTCAACTGGGCATGGAGCAGCACTATG |
| | | AACTTGGGGAGTACATCAGAAAGAGATACAGAAAATTC |
| | | TTGAATGAGTCCTACAAACATGAACAGGTTTACATCAG |
| | | AAGCACAGATGTTGACAGGACTTTGATGAGTGCCATGA |
| | | CAAACCTGGCAGCCCTGTTCCCCCCTGAAGGTGTCAGCA |
| | | TCTGGAATCCCATTCTCCTTTGGCAACCCATCCCAGTGC |
| | | ACACAGTTCCTCTTTCTGAAGATCAGTTGCTCTACCTGC |
| | | CTTTCAGGAATTGTCCAAGGTTTCAAGAACTTGAGAGTG |
| | | AAACTTTGAAATCAGAAGAATTTCAGAAGAGGCTGCAC |
| | | CCTTACAAGGATTTCATAGCCACCTTGGGAAAGCTTTCA |
| | | GGGTTGCATGGGCAAGACCTTTTTGGCATTTGGAGCAAA |
| | | GTCTATGACCCTTTATATTGTGAGAGTGTTCACAATTTC |
| | | ACCTTGCCTTCTTGGGCCACTGAGGACACCATGACAAAG |
| | | TTGAGAGAATTGTCAGAATTGTCCCTCCTGTCTCTCTAT |
| | | GGCATTCACAAGCAGAAAGAGAAATCCAGGCTCCAAGG |
| | | GGGTGTCCTGGTCAATGAAATCCTGAATCACATGAAGA |
| | | GAGCAACTCAGATCCCAAGCTACAAAAAACTCATCATG |
| | | TATTCTGCTCATGACACAACTGTGAGTGGCCTGCAGATG |
| | | GCTCTAGATGTTTACAATGGCCTCCTCCCTCCCTATGCTT |
| | | CTTGCCACTTGACAGAATTGTATTTTGAGAAGGGGGAGT |
| | | ACTTTGTGGAGATGTACTACAGGAATGAGACCCAGCAT |
| | | GAGCCTTATCCTCTCATGCTGCCTGGCTGCAGCCCCAGT |
| | | TGTCCTCTTGAGAGATTTGCTGAGCTGGTTGGCCCTGTG |
| | | ATCCCTCAGGACTGGTCAACTGAGTGCATGACAACAAA |
| | | CAGTCATCAAGGAACTGAGGACAGCACAGATTAGAGAA |
| | | CAGCGCCTCCCTGACTCTCCACCTCGAAAGAGGTGGAG |
| | | AGTCAGGGAGGCCCAGAGGGTCTTAGAGTGTCACAACA |
| | | TTTGGGCCTCTAAAAATTAGGTCATGTGGCAGAATGTTG |
| | | TGAACAGTTTTCAGATCTGGGAGCCTTGCTTTGGAGGCG |
| | | CTTTCAAAAATGATGCAGTCCATGAGTGCACAGTGCGG |
| | | GGTGATCTCTTTCTTCTTTTTGTCCCTTACTATTCCAGTA |
| | | TGCATCTTACACAACCAGCCATATTTGTCCCACACTTTaT |
| | | CTTCATACTCCCTCGAAGCTTCCCTGGTCATTTCAACATC |
| | | GATAAGCTTAATGTCCTTCCTATTtTGTGAGTCCAGAAGC |
| | | TTTCTGATGTCATCGGAGCCTTGACAGCTTAGAACCATC |
| | | CCCTGCGGAAGAGCACCTATAACTGACGAGGTCAACCC |
| | | GGGTTGCGCATTGAAGAGGTCGGCAAGATCCATGCCGT |
| | | GTGAGTACTTGGAATCTTGCTTGAATTGTTTTTGATCAA |
| | | CGGGTTCCCTGTAAAAGTGTATGAACTGCCCGTTCTGTG |
| | | GTTGGAAAATTGCTATTTCCACTGGATCATTAAATCTAC |
| | | CCTCAATGTCAATCCATGTAGGAGCGTTGGGGTCAATTC |
| | | CTCCCATGAGGTCTTTTAAAAGCATTGTCTGGCTGTAGC |
| | | TTAAGCCCACCTGAGGTGGACCTGCTGCTCCAGGCGCTG |
| | | GCCTGGGTGAgTTGACTGCAGGTTTCTCGCTTGTGAGAT |
| | | CAATTGTTGTGTTTTCCCATGCTCTCCCCACAATCGATGT |
| | | TCTACAAGCTATGTATGGCCATCCTTCACCTGAAAGGCA |
| | | AACTTTATAGAGGATGTTTTCATAAGGGTTCCTGTCCCC |
| | | AACTTGGTCTGAAACAAACATGTTGAGTTTTCTCTTGGC |
| | | CCCGAGAACTGCCTTCAAGAGaTCCTCGCTGTTGCTTGG |
| | | CTTGATCAAAATTGACTCTAACATGTTACCCCCATCCAA |
| | | CAGGGCTGCCCCTGCCTTCACGGCAGCACCAAGACTAA |
| | | AGTTATAGCCAGAAATGTTGATGCTGGACTGCTGTTCAG |
| | | TGATGACCCCCAGAACTGGGTGCTTGTCTTTCAGCCTTT |
| | | CAAGATCATTAAGATTTGGATACTTGACTGTGTAAAGCA |
| | | AGCCAAGGTCTGTGAGCGCTTGTACAACGTCATTGAGC |
| | | GGAGTCTGTGACTGTTTGGCCATACAAGCCATAGTTAGA |
| | | CTTGGCATTGTGCCAAATTGATTGTTCAAAAGTGATGAG |
| | | TCTTTCACATCCCAAACTCTTACCACACCACTTGCACCC |
| | | TGCTGAGGCTTTCTCATCCCAACTATCTGTAGGATCTGA |
| | | GATCTTTGGTCTAGTTGCTGTGTTGTTAAGTTCCCCATAT |
| | | ATACCCCTGAAGCCTGGGGCCTTTCAGACCTCATGATCT |
| | | TGGCCTTCAGCTTCTCAAGGTCAGCCGCAAGAGACATCA |
| | | GTTCTTCTGCACTGAGCCTCCCCACTTTCAAAACATTCTT |
| | | CTTTGATGTTGACTTTAAATCCACAAGAGAATGTACAGT |
| | | CTGGTTGAGACTTCTGAGTCTCTGTAGGTCTTTGTCATCT |
| | | CTCTTTTCCTTCCTCATGATCCTCTGAACATTGCTGACCT |
| | | CAGAGAAGTCCAACCCATTCAGAAGGTTGGTTGCATCCT |
| | | TAATGACAGCAGCCTTCACATCTGATGTGAAGCTCTGCA |
| | | ATTCTCTTCTCAATGCTTGCGTCCATTGGAAGCTCTTAAC |
| | | TTCCTTAGACAAGGACATCTTGTTGCTCAATGGTTTCTC |
| | | AAGACAAATGCGCAATCAAATGCctaggatccactgtgcg |
| 11 | PSA-GP-S- | gcgcaccggggatcCTAGGCTTTTTGGATTGCGCTTTCCTCTAG |
| | segment of | ATCAACTGGGTGTCAGGCCCTATCCTACAGAAGGATGT |
| | PMVS (12), | GGGTCCCTGTGGTCTTCCTCACCCTGTCTGTGACTTGGA |
| | which is a PMVS | TTGGAGCTGCCCCCCTCATCCTGTCCAGGATTGTGGGTG |
| | of artLCMV- | GCTGGGAGTGTGAGAAGCATTCCCAACCCTGGCAGGTG |
| | PAP-NP/PSA-GP | CTGGTGGCCTCCAGAGGCAGGGCTGTGTGTGGGGGGGT |
| | | CCTGGTGCACCCCCAGTGGGTCCTCACTGCTGCCCACTG |
| | | CATCAGGAACAAGAGTGTGATCTTGCTGGGGAGGCACA |
| | | GCCTGTTCCATCCTGAAGACACAGGCCAGGTCTTCCAGG |
| | | TCAGCCACAGCTTCCCCCACCCCCTCTATGACATGAGCC |
| | | TCCTGAAGAACAGATTCCTCAGGCCTGGTGATGACTCCA |
| | | GCCATGACCTCATGCTGCTCAGGCTGTCAGAGCCTGCAG |
| | | AGCTCACTGATGCTGTGAAGGTCATGGACCTGCCCACCC |
| | | AGGAGCCAGCCCTGGGGACCACCTGCTATGCCTCAGGC |
| | | TGGGGCAGCATTGAACCAGAGGAGTTCTTGACCCCCAA |
| | | GAAACTTCAGTGTGTGGACCTCCATGTCATCTCCAATGA |
| | | TGTGTGTGCCCAAGTTCACCCCCAGAAGGTCACCAAGTT |
| | | CATGCTGTGTGCTGGAAGATGGACAGGGGGCAAAAGCA |
| | | CCTGCTCTGGTGACTCTGGGGGCCCCCTTGTGTGCAATG |
| | | GTGTGCTCCAAGGCATCACCTCCTGGGGCAGTGAGCCAT |
| | | GTGCCCTGCCTGAAAGGCCTTCCCTGTACACCAAGGTGG |
| | | TTCATTACAGGAAGTGGATCAAGGACACAATTGTGGCC |
| | | AACCCCTGAAGAACAGCGCCTCCCTGACTCTCCACCTCG |
| | | AAAGAGGTGGAGAGTCAGGGAGGCCCAGAGGGTCTCA |
| | | GCGTCTTTTCCAGATAGTTTTTACACCAGGCACCTTGAA |
| | | TGCACCACAACTACAGATCCCCTTGTTGGTCAAGCGGTG |
| | | TGGCTTTGGACATGAACCGCCCTTTATGTGTCTATGTGT |
| | | TGGTATCTTCACAAGATGCAGAAAGATGCTGATTAGAT |
| | | ATGCTGATGTTGAAAACATCAAAAGATCCATTAAGGCT |
| | | AAAGGAGTACTCCCTTGTCTTTTTATGTAGTCCTTCCTCA |
| | | ACATCTCTGTGATCATGTTATCTGCTTCTTGTTCGATTTG |
| | | ATCACTAAAGTGGGTCTCATTCAAGTAGGAGCCATTAGT |
| | | GACAAGCCAGCACTTGGGTACACTAGTCTCACCAGTCTT |
| | | AGCATGTTCCAGATACCAGAACTTTGAGTAATTACAGTA |
| | | TGGTACCCCCATTAGATCTCTTAGATGATTCCTCATCAA |
| | | CAGCTGATCGGAAATCAGAGAATTTACTGTTGTTTTGAA |
| | | TACATGCAAGGCAGACTCTACATCTTGCTTGAACTTACT |
| | | CAGGGCGGCCTTGTTGTAATCAATTAGTCGTAGCATGTC |
| | | ACAGAACTCTTCATCATGATTGACATTACATTTTGCAAC |
| | | AGCTGTATTCCCAAAACATTTGAGCTCTGCAGCAAGGAT |
| | | CATCCATTTGGTCAGGCAATAACCACCTGGATTTTCTAC |
| | | TCCTGAGGAGTCTGACAGGGTCCAGGTGAATGTGCCTG |
| | | CAAGTCTCCTAGTGAGAAACTTTGTCTTTTCCTGAGCAA |
| | | AGAGGATTCTAGACATCCCAAAAGGGCCTGCATATCTA |
| | | CAGTGGTTTTCCCAAGTCCTGTTTTGTATGATTAGGTACT |
| | | GATAGCTTGTTTGGCTGCACCAAGTGGTCTTGCCATCTG |
| | | AACCTGCCCAGCCCCAGCCACTTCTCATGTATTTTCCTC |
| | | CAAAGGCAGTTCTAAACATGTCCAAGACTCTACCTCTGA |
| | | AAGTCCTACACTGGCTTATAGCGCTCTGTGGGTCCGAAA |
| | | ATGACAAGTTGTATTGAATGGTGATGCCATTGTTAAAAT |
| | | CACAAGACACTGCTTTGTGGTTGGAATTCCCTCTAATAC |
| | | TGAGGTGCAGACTCGAGACTATACTCATGAGTGTATGGT |
| | | CAAAAGTCTTTTTGTTGAAAGCGGAGGTTAAGTTGCAAA |
| | | AATTGTGATTAAGGATGGAGTCGTTAGTGAAAGTTAGCT |
| | | CCAGTCCAGAGCTTCCCATACTGATGTAGTGATGAGAGT |
| | | TGTTGGCTGAGCACGCATTGGGCATCGTCAGATTTAAGT |
| | | GAGACATATCAAACTCCACTGATTTGAACTGGTAAACCC |
| | | CTTTATAGATGTCGGGACCATTAAGGCCGTACATGCCAC |
| | | AGGACCTACCAGCCAAAAAAAGGAAGCTGACCAGTGCT |
| | | AATATCCCACAGGTGGCGAAATTGTACACAGCTTTGATG |
| | | CTCGTGATTATAATGAGCACAATAATGACAATGTTGATG |
| | | ACCTCATCAATGATGTGAGGCAAAGCCTCAAACATTGTC |
| | | ACAATCTGACCCATCTTGTTGCTCAATGGTTTCTCAAGA |
| | | CAAATGCGCAATCAAATGCctaggatccactgtgcg |
| 12 | PAP-NP-S- | gcgcaccggggatcCTAGGCATACCTTGGACGCGCATATTACTT |
| | segment of | GATCAAAGATGATAGCTGCACCCCTTCTCCTGGCCAGGG |
| | PMVS(05) | CAGCAAGCCTCAGCCTTGGCTTCTTGTTTCTGCTTTTTTT |
| | c132/05/05, which | CTGGCTGGACAGAAGTGTGCTTGCCAAGGAGTTGAAGT |
| | is a PMVS of | TTGTGACTTTGGTGTTCAGGCATGGAGACAGAAGTCCCA |
| | artPICV-PAP- | TTGACACCTTTCCCACAGACCCCATCAAGGAATCCTCAT |
| | NP/PSA-GP | GGCCTCAAGGATTTGGTCAACTCACTCAACTGGGCATGG |
| | | AGCAGCACTATGAACTTGGGGAGTACATCAGAAAGAGA |
| | | TACAGAAAATTCTTGAATGAGTCCTACAAACATGAACA |
| | | GGTTTACATCAGAAGCACAGATGTTGACAGGACTTTGAT |
| | | GAGTGCCATGACAAACCTGGCAGCCCTGTTCCCCCCTGA |
| | | AGGTGTCAGCATCTGGAATCCCATTCTCCTTTGGCAACC |
| | | CATCCCAGTGCACACAGTTCCTCTTTCTGAAGATCAGTT |
| | | GCTCTACCTGCCTTTCAGGAATTGTCCAAGGTTTCAAGA |
| | | ACTTGAGAGTGAAACTTTGAAATCAGAAGAATTTCAGA |
| | | AGAGGCTGCACCCTTACAAGGATTTCATAGCCACCTTGG |
| | | GAAAGCTTTCAGGGTTGCATGGGCAAGACCTTTTTGGCA |
| | | TTTGGAGCAAAGTCTATGACCCTTTATATTGTGAGAGTG |
| | | TTCACAATTTCACCTTGCCTTCTTGGGCCACTGAGGACA |
| | | CCATGACAAAGTTGAGAGAATTGTCAGAATTGTCCCTCC |
| | | TGTCTCTCTATGGCATTCACAAGCAGAAAGAGAAATCC |
| | | AGGCTCCAAGGGGGTGTCCTGGTCAATGAAATCCTGAA |
| | | TCACATGAAGAGAGCAACTCAGATCCCAAGCTACAAAA |
| | | AACTCATCATGTATTCTGCTCATGACACAACTGTGAGTG |
| | | GCCTGCAGATGGCTCTAGATGTTTACAATGGCCTCCTCC |
| | | CTCCCTATGCTTCTTGCCACTTGACAGAATTGTATTTTGA |
| | | GAAGGGGGAGTACTTTGTGGAGATGTACTACAGGAATG |
| | | AGACCCAGCATGAGCCTTATCCTCTCATGCTGCCTGGCT |
| | | GCAGCCCCAGTTGTCCTCTTGAGAGATTTGCTGAGCTGG |
| | | TTGGCCCTGTGATCCCTCAGGACTGGTCAACTGAGTGCA |
| | | TGACAACAAACAGTCATCAAGGAACTGAGGACAGCACA |
| | | GATTAGGCCCTAGCCTCGACATGGGCCTCGACGTCACTC |
| | | CCCAATAGGGGAGTGACGTCGAGGCCTCTGAGGACTTG |
| | | AGCTCAGAGGTTGATCAGATCTGTGTTGTTCCTGTACAG |
| | | CGTGTCAATAGGCAAGCATCTCATCGGCTTCTGGTCCCT |
| | | AACCCAGCCTGTCACTGTTGCATCAAACATGATGGTATC |
| | | AAGCAATGCACAGTGAGGATTCGCAGTGGTTTGTGCAG |
| | | CCCCCTTCTTCTTCTTCTTTATGACCAAACCTTTATGTTT |
| | | GGTGCAGAGTAGATTGTATCTCTCCCAGATCTCATCCTC |
| | | AAAGGTGCGTGCTTGCTCGGCACTGAGTTTCACGTCAAG |
| | | CACTTTTAAGTCTCTTCTCCCATGCATTTCGAACAAACT |
| | | GATTATATCATCTGAACCTTGAGCAGTGAAAACCATGTT |
| | | TTGAGGTAAATGTCTGATGATTGAGGAAATCAGGCCTG |
| | | GTTGGGCATCAGCCAAGTCCTTTAAAAGgAGACCATGTG |
| | | AGTACTTGCTTTGCTCTTTGAAGGACTTCTCATCGTGGG |
| | | GAAATCTGTAACAATGTATGTAGTTGCCCGTGTCAGGCT |
| | | GGTAGATGGCCATTTCCACCGGATCATTTGGTGTTCCTT |
| | | CAATGTCAATCCATGTGGTAGCTTTTGAATCAAGCATCT |
| | | GAATTGAGGACACAACAGTaTCTTCTTTCTCCTTAGGGA |
| | | TTTGTTTAAGGTCCGGTGATCCTCCGTTTCTTACTGGTGG |
| | | CTGGATAGCACTCGGCTTCGAATCTAAATCTACAGTGGT |
| | | GTTATCCCAAGCCCTCCCTTGAACTTGAGACCTTGAGCC |
| | | AATGTAAGGCCAACCATCCCCTGAAAGACAAATCTTGT |
| | | ATAGTAAATTTTCATAAGGATTTCTCTGTCCGGGTGTAG |
| | | TGCTCACAAACATACCTTCACGATTCTTTATTTGCAATA |
| | | GACTCTTTATGAGAGTACTAAACATAGAAGGCTTCACCT |
| | | GGATGGTCTCAAGCATATTGCCACCATCAATCATGCAAG |
| | | CAGCTGCTTTGACTGCTGCAGACAAACTGAGATTGTACC |
| | | CTGAGATGTTTATGGCTGATGGCTCATTACTAATGATTT |
| | | TTAGGGCACTGTGTTGCTGTGTGAGTTTCTCTAGATCTG |
| | | TCATGTTCGGGAACTTGACAGTGTAGAGCAAACCAAGT |
| | | GCACTCAGCGCTTGGACAACATCATTAAGTTGTTCACCC |
| | | CCTTGCTCAGTCATACAAGCGATGGTTAAGGCTGGCATT |
| | | GATCCAAATTGATTGATCAACAATGTATTATCCTTGATG |
| | | TCCCAGATCTTCACAACCCCATCTCTGTTGCCTGTGGGT |
| | | CTAGCATTAGCGAACCCCATTGAGCGAAGGATTTCGGCT |
| | | CTTTGTTCCAACTGAGTGTTTGTGAGATTGCCCCCATAA |
| | | ACACCAGGCTGAGACAAACTCTCAGTTCTAGTGACTTTC |
| | | TTTCTTAACTTGTCCAAATCAGATGCAAGCTCCATTAGC |
| | | TCCTCTTTGGCTAAGCCTCCCACCTTAAGCACATTGTCC |
| | | CTCTGGATTGATCTCATATTCATCAGAGCATCAACCTCT |
| | | TTGTTCATGTCTCTTAACTTGGTCAGATCAGAATCAGTC |
| | | CTTTTATCTTTGCGCATCATTCTTTGAACTTGAGCAACTT |
| | | TGTGAAAGTCAAGAGCAGATAACAGTGCTCTTGTGTCC |
| | | GACAACACATCAGCCTTCACAGGATGGGTCCAGTTGGA |
| | | TAGACCCCTCCTAAGGGACTGTACCCAGCGGAATGATG |
| | | GGATGTTGTCAGACATTTTGGGGTTGTTTGCACTTCCTC |
| | | CGAGTCAGTGAAGAAGTGAACGTACAGCGTGATCTAGA |
| | | ATCGCctaggatccactgtgcg |
| 13 | PSA-GP-S- | gcgcaccggggatcCTAGGCATACCTTGGACGCGCATATTACTT |
| | segment of | GATCAAAGATGTGGGTCCCTGTGGTCTTCCTCACCCTGT |
| | PMVS(05) | CTGTGACTTGGATTGGAGCTGCCCCCCTCATCCTGTCCA |
| | c132/05/05, which | GGATTGTGGGTGGCTGGGAGTGTGAGAAGCATTCCCAA |
| | is a PMVS of | CCCTGGCAGGTGCTGGTGGCCTCCAGAGGCAGGGCTGT |
| | artPICV-PAP- | GTGTGGGGGGGTCCTGGTGCACCCCCAGTGGGTCCTCAC |
| | NP/PSA-GP | TGCTGCCCACTGCATCAGGAACAAGAGTGTGATCTTGCT |
| | | GGGGAGGCACAGCCTGTTCCATCCTGAAGACACAGGCC |
| | | AGGTCTTCCAGGTCAGCCACAGCTTCCCCCACCCCCTCT |
| | | ATGACATGAGCCTCCTGAAGAACAGATTCCTCAGGCCT |
| | | GGTGATGACTCCAGCCATGACCTCATGCTGCTCAGGCTG |
| | | TCAGAGCCTGCAGAGCTCACTGATGCTGTGAAGGTCAT |
| | | GGACCTGCCCACCCAGGAGCCAGCCCTGGGGACCACCT |
| | | GCTATGCCTCAGGCTGGGGCAGCATTGAACCAGAGGAG |
| | | TTCTTGACCCCCAAGAAACTTCAGTGTGTGGACCTCCAT |
| | | GTCATCTCCAATGATGTGTGTGCCCAAGTTCACCCCCAG |
| | | AAGGTCACCAAGTTCATGCTGTGTGCTGGAAGATGGAC |
| | | AGGGGGCAAAAGCACCTGCTCTGGTGACTCTGGGGGTC |
| | | CCCTTGTGTGCAATGGTGTGCTCCAAGGCATCACCTCCT |
| | | GGGGCAGTGAGCCATGTGCCCTGCCTGAAAGGCCTTCC |
| | | CTGTACACCAAGGTGGTTCATTACAGGAAGTGGATCAA |
| | | GGACACAATTGTGGCCAACCCCTGAGCCCTAGCCTCGA |
| | | CATGGGCCTCGACGTCACTCCCCAATAGGGGAGTGACG |
| | | TCGAGGCCTCTGAGGACTTGAGCTTATTTACCCAGTCTC |
| | | ACCCATTTGTAGGGTTTCTTTGGGATTTTATAATACCCA |
| | | CAGCTGCAAAGAGAGTTCCTAGTAATCCTATGTGGCTTC |
| | | GGACAGCCATCACCAATGATGTGCCTATGAGTGGGTATT |
| | | CCAACTAAGTGGAGAAACACTGTGATGGTGTAAAACAC |
| | | CAAAGACCAGAAGCAAATGTCTGTCAATGCTAGTGGAG |
| | | TCTTACCTTGTCTTTCTTCATATTCTTTTATCAGCATTTCA |
| | | TTGTACAGATTCTGGCTCTCCCACAACCAATCATTCTTA |
| | | AAATGCGTTTCATTGAGGTACGAGCCATTGTGAACTAAC |
| | | CAACACTGCGGTAAAGAATGTCTcCCTGTGATGGTATCA |
| | | TTGATGTACCAAAATTTTGTATAGTTGCAATAAGGGATT |
| | | TTGGCAAGCTGTTTGAGACTGTTTCTAATCACAAGTGAG |
| | | TCAGAAATAAGTCCGTTGATAGTCTTTTTAAAGAGATTC |
| | | AACGAATTCTCAACATTAAGTTGTAAGGTTTTGATAGCA |
| | | TTCTGATTGAAATCAAATAACCTCATCGTATCGCAAAAT |
| | | TCTTCATTGTGATCTTTGTTGCATTTTGCCATCACAGTGT |
| | | TATCAAAACATTTTATTCCAGCCCAAACAATAGCCCATT |
| | | GCTCCAAACAGTAACCACCTGGGACATGTTGCCCAGTA |
| | | GAGTCACTCAAGTCCCAAGTGAAAAAGCCAAGGAGTTT |
| | | CCTGCTCACAGAACTATAAGCAGTTTTTTGGAGAGCCAT |
| | | CCTTATTGTTGCCATtGGAGTATATGTACAGTGATTTTCC |
| | | CATGTGGTGTTCTGTATGATCAGGAAATTGTAATGTGTC |
| | | CCACCTTCACAGTTTGTTAGTCTGCAAGACCCTCCACTA |
| | | CAGTTATTGAAACATTTTCCAACCCACGCAATTTTTGGG |
| | | TCCCCAATGATTTGAGCAAGCGACGCAATAAGATGTCT |
| | | GCCAACCTCACCTCCTCTATCCCCAACTGTCAAGTTGTA |
| | | CTGGATCAACACCCCAGCACCCTCAACTGTTTTGCATCT |
| | | GGCACCTACATGACGAGTGACATGGAGCACATTGAAGT |
| | | GTAACTCATTAAGCAACCATTTTAATGTGTGACCTGCTT |
| | | CTTCTGTCTTATCACAATTACTAATGTTACCATATGCAA |
| | | GGCTTCTGATGTTGGAAAAGTTTCCAGTAGTTTCATTTG |
| | | CAATGGATGTGTTTGTCAAAGTGAGTTCAATTCCCCATG |
| | | TTGTGTTAGATGGTCCTTTGTAGTAATGATGTGTGTTGTT |
| | | CTTGCTACATGATTGTGGCAAGTTGTCAAACATTCTTGT |
| | | GAGGTTGAACTCAACGTGGGTGAGATTGTGCCTCCTATC |
| | | AATCATCATGCCATCACAACTTCTGCCAGCCAAAATGAG |
| | | GAAGGTGATGAGTTGGAATAGGCCACATCTCATCAGAT |
| | | TGACAAATCCTTTGATGATGCATAGGGTTGAGACAATG |
| | | ATTAAGGCGACATTGAACACCTCCTGCAGGACTTCGGGT |
| | | ATAGACTGGATCAAAGTCACAACTTGTCCCATTTTGGGG |
| | | TTGTTTGCACTTCCTCCGAGTCAGTGAAGAAGTGAACGT |
| | | ACAGCGTGATCTAGAATCGCctaggatccactgtgcg |
| 14 | PSMA2-NP-S- | gcgcaccggggatcCTAGGCTTTTTGGATTGCGCTTTCCTCTAG |
| | segment of | ATCAACTGGGTGTCAGGCCCTATCCTACAGAAGGATGC |
| | PMVS (09) c19/ | ACATCCATTCAACCAATGAAGTGACAAGAATTTACAAT |
| | 7/2, which is a | GTGATTGGAACTCTCAGAGGAGCAGTGGAACCAGACAG |
| | PMVS of | ATATGTCATTCTGGGAGGTCACAGGGACTCCTGGGTGTT |
| | artLCMV- | TGGTGGAATTGACCCTCAGAGTGGAGCAGCTGTGGTTC |
| | PSMA2- | ATGAAATTGTCAGGAGTTTTGGAACACTGAAAAAGGAA |
| | NP/PSMA1-GP | GGGTGGAGACCCAGAAGAACAATTTTGTTTGCAAGCTG |
| | | GGATGCAGAAGAATTTGGTCTTCTTGGTTCAACTGAGTG |
| | | GGCAGAGGAGAACTCAAGACTCCTTCAGGAGAGAGGAG |
| | | TAGCTTACATCAATGCTGACTCATCTATTGAAGGAAATT |
| | | ACACTCTGAGAGTTGATTGCACTCCACTAATGTACAGCT |
| | | TGGTTCACAATCTGACAAAAGAGCTGAAAAGCCCTGAT |
| | | GAAGGGTTTGAAGGAAAATCTCTTTATGAAAGTTGGAC |
| | | AAAAAAAAGTCCCTCCCCAGAGTTCAGTGGAATGCCCA |
| | | GGATCAGCAAATTGGGATCTGGAAATGATTTTGAGGTG |
| | | TTCTTCCAAAGACTTGGAATTGCTTCAGGCAGAGCAAGG |
| | | TACACCAAGAATTGGGAAACCAACAAATTCAGTGGTTA |
| | | TCCACTATATCACAGTGTTTATGAAACATATGAGTTGGT |
| | | GGAAAAGTTTTATGATCCAATGTTCAAATATCATCTGAC |
| | | TGTGGCACAGGTCAGAGGAGGGATGGTGTTTGAGCTGG |
| | | CCAATTCCATAGTTCTCCCTTTTGATTGCAGAGATTATG |
| | | CTGTGGTTTTGAGAAAGTATGCTGACAAAATTTACAGCA |
| | | TTTCAATGAAACATCCACAGGAAATGAAGACATACAGT |
| | | GTCTCATTTGATTCACTTTTTTCTGCAGTGAAGAATTTCA |
| | | CAGAAATTGCTTCCAAGTTCAGTGAAAGGCTTCAGGACT |
| | | TTGACAAAAGCAACCCAATTGTTTTGAGAATGATGAAT |
| | | GATCAACTCATGTTTCTGGAAAGAGCATTCATTGATCCC |
| | | TTGGGGTTGCCAGACAGGCCTTTTTACAGGCATGTCATC |
| | | TATGCCCCAAGCAGTCACAACAAGTATGCAGGGGAGTC |
| | | ATTTCCAGGAATTTATGATGCTCTGTTTGACATTGAAAG |
| | | CAAAGTGGACCTTTCCAAGGCCTGGGGAGAAGTGAAGA |
| | | GACAGATTTATGTTGCAGCCTTCACAGTTCAGGCAGCTG |
| | | CAGAGACTTTGAGTGAAGTTGCTTAAAGAACAGCGCCT |
| | | CCCTGACTCTCCACCTCGAAAGAGGTGGAGAGTCAGGG |
| | | AGGCCCAGAGGGTCTTAGAGTGTCACAACATTTGGGCC |
| | | TCTAAAAATTAGGTCATGTGGCAGAATGTTGTGAACAGT |
| | | TTTCAGATCTGGGAGCCTTGCTTTGGAGGCGCTTTCAAA |
| | | AATGATGCAGTCCATGAGTGCACAGTGCGGGGTGATCT |
| | | CTTTCTTCTTTTTGTCCCTTACTATTCCAGTATGCATCTT |
| | | ACACAACCAGCCATATTTGTCCCACACTTTaTCTTCATAC |
| | | TCCCTCGAAGCTTCCCTGGTCATTTCAACATCGATAAGC |
| | | TTAATGTCCTTCCTATTtTGTGAGTCCAGAAGCTTTCTGA |
| | | TGTCATCGGAGCCTTGACAGCTTAGAACCATCCCCTGCG |
| | | GAAGAGCACCTATAACTGACGAGGTCAACCCGGGTTGC |
| | | GCATTGAAGAGGTCGGCAAGATCCATGCCGTGTGAGTA |
| | | CTTGGAATCTTGCTTGAATTGTTTTTGATCAACGGGTTCC |
| | | CTGTAAAAGTGTATGAACTGCCCGTTCTGTGGTTGGAAA |
| | | ATTGCTATTTCCACTGGATCATTAAATCTACCCTCAATG |
| | | TCAATCCATGTAGGAGCGTTGGGGTCAATTCCTCCCATG |
| | | AGGTCTTTTAAAAGCATTGTCTGGCTGTAGCTTAAGCCC |
| | | ACCTGAGGTGGACCTGCTGCTCCAGGCGCTGGCCTGGGT |
| | | GAgTTGACTGCAGGTTTCTCGCTTGTGAGATCAATTGTT |
| | | GTGTTTTCCCATGCTCTCCCCACAATCGATGTTCTACAA |
| | | GCTATGTATGGCCATCCTTCACCTGAAAGGCAAACTTTA |
| | | TAGAGGATGTTTTCATAAGGGTTCCTGTCCCCAACTTGG |
| | | TCTGAAACAAACATGTTGAGTTTTCTCTTGGCCCCGAGA |
| | | ACTGCCTTCAAGAGaTCCTCGCTGTTGCTTGGCTTGATCA |
| | | AAATTGACTCTAACATGTTACCCCCATCCAACAGGGCTG |
| | | CCCCTGCCTTCACGGCAGCACCAAGACTAAAGTTATAGC |
| | | CAGAAATGTTGATGCTGGACTGCTGTTCAGTGATGACCC |
| | | CCAGAACTGGGTGCTTGTCTTTCAGCCTTTCAAGATCAT |
| | | TAAGATTTGGATACTTGACTGTGTAAAGCAAGCCAAGG |
| | | TCTGTGAGCGCTTGTACAACGTCATTGAGCGGAGTCTGT |
| | | GACTGTTTGGCCATACAAGCCATAGTTAGACTTGGCATT |
| | | GTGCCAAATTGATTGTTCAAAAGTGATGAGTCTTTCACA |
| | | TCCCAAACTCTTACCACACCACTTGCACCCTGCTGAGGC |
| | | TTTCTCATCCCAACTATCTGTAGGATCTGAGATCTTTGGT |
| | | CTAGTTGCTGTGTTGTTAAGTTCCCCATATATACCCCTG |
| | | AAGCCTGGGGCCTTTCAGACCTCATGATCTTGGCCTTCA |
| | | GCTTCTCAAGGTCAGCCGCAAGAGACATCAGTTCTTCTG |
| | | CACTGAGCCTCCCCACTTTCAAAACATTCTTCTTTGATGT |
| | | TGACTTTAAATCCACAAGAGAATGTACAGTCTGGTTGAG |
| | | ACTTCTGAGTCTCTGTAGGTCTTTGTCATCTCTCTTTTCC |
| | | TTCCTCATGATCCTCTGAACATTGCTGACCTCAGAGAAG |
| | | TCCAACCCATTCAGAAGGTTGGTTGCATCCTTAATGACA |
| | | GCAGCCTTCACATCTGATGTGAAGCTCTGCAATTCTCTT |
| | | CTCAATGCTTGCGTCCATTGGAAGCTCTTAACTTCCTTA |
| | | GACAAGGACATCTTGTTGCTCAATGGTTTCTCAAGACAA |
| | | ATGCGCAATCAAATGCctaggatccactgtgcg |
| 15 | PSMA1-GP-S- | gcgcaccggggatcCTAGGCTTTTTGGATTGCGCTTTCCTCTAG |
| | segment of | ATCAACTGGGTGTCAGGCCCTATCCTACAGAAGGATGT |
| | PMVS (09) c19/ | GGAATCTTCTTCATGAAACTGACTCAGCTGTGGCCACAG |
| | 7/2, which is a | CCAGAAGACCCAGGTGGCTGTGTGCAGGGGCCCTTGTT |
| | PMVS of | CTTGCAGGTGGTTTTTTTCTCCTTGGCTTCCTCTTTGGTT |
| | artLCMV- | GGTTCATCAAGTCTTCAAATGAAGCAACCAACATCACTC |
| | PSMA2- | CAAAGCACAACATGAAAGCATTTTTGGATGAATTGAAA |
| | NP/PSMA1-GP | GCTGAGAACATCAAGAAGTTTTTGCACAATTTCACACAG |
| | | ATTCCACATTTGGCAGGAACAGAACAAAACTTTCAGCTT |
| | | GCAAAGCAAATTCAATCCCAGTGGAAAGAATTTGGTCT |
| | | GGATTCTGTTGAGCTGGCTCATTATGATGTTCTGTTGTCC |
| | | TACCCAAACAAGACTCATCCCAACTACATCTCAATCATC |
| | | AATGAAGATGGAAATGAGATTTTCAACACCTCTTTGTTT |
| | | GAACCACCACCTCCAGGATATGAAAATGTGTCAGACAT |
| | | TGTTCCTCCTTTCAGTGCTTTTTCTCCTCAAGGCATGCCA |
| | | GAGGGAGATCTGGTCTATGTCAACTATGCAAGAACTGA |
| | | AGACTTTTTCAAATTGGAAAGAGACATGAAAATCAATT |
| | | GCTCTGGGAAAATTGTCATTGCCAGATATGGGAAAGTTT |
| | | TCAGAGGCAACAAGGTGAAAAATGCCCAGCTGGCAGGT |
| | | GCCAAAGGAGTCATTCTCTACTCTGACCCTGCAGACTAT |
| | | TTTGCTCCTGGGGTGAAATCTTATCCTGATGGTTGGAAT |
| | | CTTCCTGGAGGTGGTGTCCAGAGGGGCAACATCCTCAAT |
| | | CTGAATGGTGCAGGAGATCCACTCACCCCAGGTTACCC |
| | | AGCAAATGAATATGCTTACAGAAGAGGAATTGCAGAGG |
| | | CTGTTGGTCTTCCCAGCATTCCTGTTCATCCAATTGGATA |
| | | CTATGATGCCCAGAAACTCCTGGAAAAGATGGGTGGTT |
| | | CAGCACCCCCAGACAGCAGCTGGAGAGGCAGTCTCAAA |
| | | GTGCCATACAATGTTGGCCCTGGTTTCACAGGAAACTTT |
| | | TCCACTCAAAAAGTCAAATGAAGAACAGCGCCTCCCTG |
| | | ACTCTCCACCTCGAAAGAGGTGGAGAGTCAGGGAGGCC |
| | | CAGAGGGTCTCAGCGTCTTTTCCAGATAGTTTTTACACC |
| | | AGGCACCTTGAATGCACCACAACTACAGATCCCCTTGTT |
| | | GGTCAAGCGGTGTGGCTTTGGACATGAACCGCCCTTTAT |
| | | GTGTCTATGTGTTGGTATCTTCACAAGATGCAGAAAGAT |
| | | GCTGATTAGATATGCTGATGTTGAAAACATCAAAAGAT |
| | | CCATTAAGGCTAAAGGAGTACTCCCTTGTCTTTTTATGT |
| | | AGTCCTTCCTCAACATCTCTGTGATCATGTTATCTGCTTC |
| | | TTGTTCGATTTGATCACTAAAGTGGGTCTCATTCAAGTA |
| | | GGAGCCATTAGTGACAAGCCAGCACTTGGGTACACTAG |
| | | TCTCACCAGTCTTAGCATGTTCCAGATACCAGAACTTTG |
| | | AGTAATTACAGTATGGTACCCCCATTAGATCTCTTAGAT |
| | | GATTCCTCATCAACAGCTGATCGGAAATCAGAGAATTTA |
| | | CTGTTGTTTTGAATACATGCAAGGCAGACTCTACATCTT |
| | | GCTTGAACTTACTCAGGGCGGCCTTGTTGTAATCAATTA |
| | | GTCGTAGCATGTCACAGAACTCTTCATCATGATTGACAT |
| | | TACATTTTGCAACAGCTGTATTCCCAAAACATTTGAGCT |
| | | CTGCAGCAAGGATCATCCATTTGGTCAGGCAATAACCA |
| | | CCTGGATTTTCTACTCCTGAGGAGTCTGACAGGGTCCAG |
| | | GTGAATGTGCCTGCAAGTCTCCTAGTGAGAAACTTTGTC |
| | | TTTTCCTGAGCAAAGAGGATTCTAGACATCCCAAAAGG |
| | | GCCTGCATATCTACAGTGGTTTTCCCAAGTCCTGTTTTGT |
| | | ATGATTAGGTACTGATAGCTTGTTTGGCTGCACCAAGTG |
| | | GTCTTGCCATCTGAACCTGCCCAGCCCCAGCCACTTCTC |
| | | ATGTATTTTCCTCCAAAGGCAGTTCTAAACATGTCCAAG |
| | | ACTCTACCTCTGAAAGTCCTACACTGGCTTATAGCGCTC |
| | | TGTGGGTCCGAAAATGACAAGTTGTATTGAATGGTGAT |
| | | GCCATTGTTAAAATCACAAGACACTGCTTTGTGGTTGGA |
| | | ATTCCCTCTAATACTGAGGTGCAGACTCGAGACTATACT |
| | | CATGAGTGTATGGTCAAAAGTCTTTTTGTTGAAAGCGGA |
| | | GGTTAAGTTGCAAAAATTGTCATTAAGTATGGAGTCGTT |
| | | AGTGAAAGTTAGCTCCAGTCCAGAGCTTCCCATACTGAT |
| | | GTAGTGATGAGAGTTGTTGGCTGAGCACGCATTGGGCA |
| | | TCGTCAGATTTAAGTGAGACATATCAAACTCCACTGATT |
| | | TGAACTGGTAAACCCCTTTATAGATGTCGGGACCATTAA |
| | | GGCCGTACATGCCACAGGACCTACCAGCCAAAAAAAGG |
| | | AAGCTGACCAGTGCTAATATCCCACAGGTGGCGAAATT |
| | | GTACACAGCTTTGATGCTCGTGATTATAATGAGCACAAT |
| | | AATGACAATGTTGATGACCTCATCAATGATGTGAGGCA |
| | | AAGCCTCAAACATTGTCACAATCTGACCCATCTTGTTGC |
| | | TCAATGGTTTCTCAAGACAAATGCGCAATCAAATGCctag |
| | | gatccactgtgcg |
| 16 | PSMA1-NP-S- | gcgcaccggggatcCTAGGCATACCTTGGACGCGCATATTACTT |
| | segment of | GATCAAAGATGTGGAATCTTCTTCATGAAACTGACTCAG |
| | PMVS(26), which | CTGTGGCCACAGCCAGAAGACCCAGGTGGCTGTGTGCA |
| | is a PMVS of | GGGGCCCTTGTTCTTGCAGGTGGTTTTTTTCTCCTTGGCT |
| | artPICV-PSMA1- | TCCTCTTTGGTTGGTTCATCAAGTCTTCAAATGAAGCAA |
| | NP/PSMA2-GP | CCAACATCACTCCAAAGCACAACATGAAAGCATTTTTG |
| | | GATGAATTGAAAGCTGAGAACATCAAGAAGTTTTTGCA |
| | | CAATTTCACACAGATTCCACATTTGGCAGGAACAGAAC |
| | | AAAACTTTCAGCTTGCAAAGCAAATTCAATCCCAGTGG |
| | | AAAGAATTTGGTCTGGATTCTGTTGAGCTGGCTCATTAT |
| | | GATGTTCTGTTGTCCTACCCAAACAAGACTCATCCCAAC |
| | | TACATCTCAATCATCAATGAAGATGGAAATGAGATTTTC |
| | | AACACCTCTTTGTTTGAACCACCACCTCCAGGATATGAA |
| | | AATGTGTCAGACATTGTTCCTCCTTTCAGTGCTTTTTCTC |
| | | CTCAAGGCATGCCAGAGGGAGATCTGGTCTATGTCAAC |
| | | TATGCAAGAACTGAAGACTTTTTCAAATTGGAAAGAGA |
| | | CATGAAAATCAATTGCTCTGGGAAAATTGTCATTGCCAG |
| | | ATATGGGAAAGTTTTCAGAGGCAACAAGGTGAAAAATG |
| | | CCCAGCTGGCAGGTGCCAAAGGAGTCATTCTCTACTCTG |
| | | ACCCTGCAGACTATTTTGCTCCTGGGGTGAAATCTTATC |
| | | CTGATGGTTGGAATCTTCCTGGAGGTGGTGTCCAGAGGG |
| | | GCAACATCCTCAATCTGAATGGTGCAGGAGATCCACTC |
| | | ACCCCAGGTTACCCAGCAAATGAATATGCTTACAGAAG |
| | | AGGAATTGCAGAGGCTGTTGGTCTTCCCAGCATTCCTGT |
| | | TCATCCAATTGGATACTATGATGCCCAGAAACTCCTGGA |
| | | AAAGATGGGTGGTTCAGCACCCCCAGACAGCAGCTGGA |
| | | GAGGCAGTCTCAAAGTGCCATACAATGTTGGCCCTGGTT |
| | | TCACAGGAAACTTTTCCACTCAAAAAGTCAAAtgaGCCCT |
| | | AGCCTCGACATGGGCCTCGACGTCACTCCCCAATAGGG |
| | | GAGTGACGTCGAGGCCTCTGAGGACTTGAGCTCAGAGG |
| | | TTGATCAGATCTGTGTTGTTCCTGTACAGCGTGTCAATA |
| | | GGCAAGCATCTCATCGGCTTCTGGTCCCTAACCCAGCCT |
| | | GTCACTGTTGCATCAAACATGATGGTATCAAGCAATGCA |
| | | CAGTGAGGATTCGCAGTGGTTTGTGCAGCCCCCTTCTTC |
| | | TTCTTCTTTATGACCAAACCTTTATGTTTGGTGCAGAGTA |
| | | GATTGTATCTCTCCCAGATCTCATCCTCAAAGGTGCGTG |
| | | CTTGCTCGGCACTGAGTTTCACGTCAAGCACTTTTAAGT |
| | | CTCTTCTCCCATGCATTTCGAACAAACTGATTATATCAT |
| | | CTGAACCTTGAGCAGTGAAAACCATGTTTTGAGGTAAAT |
| | | GTCTGATGATTGAGGAAATCAGGCCTGGTTGGGCATCA |
| | | GCCAAGTCCTTTAAAAGgAGACCATGTGAGTACTTGCTT |
| | | TGCTCTTTGAAGGACTTCTCATCGTGGGGAAATCTGTAA |
| | | CAATGTATGTAGTTGCCCGTGTCAGGCTGGTAGATGGCC |
| | | ATTTCCACCGGATCATTTGGTGTTCCTTCAATGTCAATCC |
| | | ATGTGGTAGCTTTTGAATCAAGCATCTGAATTGAGGACA |
| | | CAACAGTaTCTTCTTTCTCCTTAGGGATTTGTTTAAGGTC |
| | | CGGTGATCCTCCGTTTCTTACTGGTGGCTGGATAGCACT |
| | | CGGCTTCGAATCTAAATCTACAGTGGTGTTATCCCAAGC |
| | | CCTCCCTTGAACTTGAGACCTTGAGCCAATGTAAGGCCA |
| | | ACCATCCCCTGAAAGACAAATCTTGTATAGTAAATTTTC |
| | | ATAAGGATTTCTCTGTCCGGGTGTAGTGCTCACAAACAT |
| | | ACCTTCACGATTCTTTATTTGCAATAGACTCTTTATGAG |
| | | AGTACTAAACATAGAAGGCTTCACCTGGATGGTCTCAA |
| | | GCATATTGCCACCATCAATCATGCAAGCAGCTGCTTTGA |
| | | CTGCTGCAGACAAACTGAGATTGTACCCTGAGATGTTTA |
| | | TGGCTGATGGCTCATTACTAATGATTTTTAGGGCACTGT |
| | | GTTGCTGTGTGAGTTTCTCTAGATCTGTCATGTTCGGGA |
| | | ACTTGACAGTGTAGAGCAAACCAAGTGCACTCAGCGCT |
| | | TGGACAACATCATTAAGTTGTTCACCCCCTTGCTCAGTC |
| | | ATACAAGCGATGGTTAAGGCTGGCATTGATCCAAATTG |
| | | ATTGATCAACAATGTATTATCCTTGATGTCCCAGATCTT |
| | | CACAACCCCATCTCTGTTGCCTGTGGGTCTAGCATTAGC |
| | | GAACCCCATTGAGCGAAGGATTTCGGCTCTTTGTTCCAA |
| | | CTGAGTGTTTGTGAGATTGCCCCCATAAACACCAGGCTG |
| | | AGACAAACTCTCAGTTCTAGTGACTTTCTTTCTTAACTTG |
| | | TCCAAATCAGATGCAAGCTCCATTAGCTCCTCTTTGGCT |
| | | AAGCCTCCCACCTTAAGCACATTGTCCCTCTGGATTGAT |
| | | CTCATATTCATCAGAGCATCAACCTCTTTGTTCATGTCTC |
| | | TTAACTTGGTCAGATCAGAATCAGTCCTTTTATCTTTGC |
| | | GCATCATTCTTTGAACTTGAGCAACTTTGTGAAAGTCAA |
| | | GAGCAGATAACAGTGCTCTTGTGTCCGACAACACATCA |
| | | GCCTTCACAGGATGGGTCCAGTTGGATAGACCCCTCCTA |
| | | AGGGACTGTACCCAGCGGAATGATGGGATGTTGTCAGA |
| | | CATTTTGGGGTTGTTTGCACTTCCTCCGAGTCAGTGAAG |
| | | AAGTGAACGTACAGCGTGATCTAGAATCGCctaggatccactgt |
| | | gcg |
| 17 | PSMA2-GP-S- | gcgcaccggggatcCTAGGCATACCTTGGACGCGCATATTACTT |
| | segment of | GATCAAAGATGCACATCCATTCAACCAATGAAGTGACA |
| | PMVS(26), which | AGAATTTACAATGTGATTGGAACTCTCAGAGGAGCAGT |
| | is a PMVS of | GGAACCAGACAGATATGTCATTCTGGGAGGTCACAGGG |
| | artPICV-PSMA1- | ACTCCTGGGTGTTTGGTGGAATTGACCCTCAGAGTGGAG |
| | NP/PSMA2-GP | CAGCTGTGGTTCATGAAATTGTCAGGAGTTTTGGAACAC |
| | | TGAAAAAGGAAGGGTGGAGACCCAGAAGAACAATTTTG |
| | | TTTGCAAGCTGGGATGCAGAAGAATTTGGTCTTCTTGGT |
| | | TCAACTGAGTGGGCAGAGGAGAACTCAAGACTCCTTCA |
| | | GGAGAGAGGAGTAGCTTACATCAATGCTGACTCATCTA |
| | | TTGAAGGAAATTACACTCTGAGAGTTGATTGCACTCCAC |
| | | TAATGTACAGCTTGGTTCACAATCTGACAAAAGAGCTG |
| | | AAAAGCCCTGATGAAGGGTTTGAAGGAAAATCTCTTTA |
| | | TGAAAGTTGGACAAAAAAAAGTCCCTCCCCAGAGTTCA |
| | | GTGGAATGCCCAGGATCAGCAAATTGGGATCTGGAAAT |
| | | GATTTTGAGGTGTTCTTCCAAAGACTTGGAATTGCTTCA |
| | | GGCAGAGCAAGGTACACCAAGAATTGGGAAACCAACA |
| | | AATTCAGTGGTTATCCACTATATCACAGTGTTTATGAAA |
| | | CATATGAGTTGGTGGAAAAGTTTTATGATCCAATGTTCA |
| | | AATATCATCTGACTGTGGCACAGGTCAGAGGAGGGATG |
| | | GTGTTTGAGCTGGCCAATTCCATAGTTCTCCCTTTTGATT |
| | | GCAGAGATTATGCTGTGGTTTTGAGAAAGTATGCTGACA |
| | | AAATTTACAGCATTTCAATGAAACATCCACAGGAAATG |
| | | AAGACATACAGTGTCTCATTTGATTCACTTTTTTCTGCA |
| | | GTGAAGAATTTCACAGAAATTGCTTCCAAGTTCAGTGAA |
| | | AGGCTTCAGGACTTTGACAAAAGCAACCCAATTGTTTTG |
| | | AGAATGATGAATGATCAACTCATGTTTCTGGAAAGAGC |
| | | ATTCATTGATCCCTTGGGGTTGCCAGACAGGCCTTTTTA |
| | | CAGGCATGTCATCTATGCCCCAAGCAGTCACAACAAGT |
| | | ATGCAGGGGAGTCATTTCCAGGAATTTATGATGCTCTGT |
| | | TTGACATTGAAAGCAAAGTGGACCCTTCCAAGGCCTGG |
| | | GGAGAAGTGAAGAGACAGATTTATGTTGCAGCCTTCAC |
| | | AGTTCAGGCAGCTGCAGAGACTTTGAGTGAAGTTGCTTA |
| | | AGCCCTAGCCTCGACATGGGCCTCGACGTCACTCCCCAA |
| | | TAGGGGAGTGACGTCGAGGCCTCTGAGGACTTGAGCTT |
| | | ATTTACCCAGTCTCACCCATTTGTAGGGTTTCTTTGGGAT |
| | | TTTATAATACCCACAGCTGCAAAGAGAGTTCCTAGTAAT |
| | | CCTATGTGGCTTCGGACAGCCATCACCAATGATGTGCCT |
| | | ATGAGTGGGTATTCCAACTAAGTGGAGAAACACTGTGA |
| | | TGGTGTAAAACACCAAAGACCAGAAGCAAATGTCTGTC |
| | | AATGCTAGTGGAGTCTTACCTTGTCTTTCTTCATATTCTT |
| | | TTATCAGCATTTCATTGTACAGATTCTGGCTCTCCCACA |
| | | ACCAATCATTCTTAAAATGCGTTTCATTGAGGTACGAGC |
| | | CATTGTGAACTAACCAACACTGCGGTAAAGAATGTCTcC |
| | | CTGTGATGGTATCATTGATGTACCAAAATTTTGTATAGT |
| | | TGCAATAAGGGATTTTGGCAAGCTGTTTGAGACTGTTTC |
| | | TAATCACAAGTGAGTCAGAAATAAGTCCGTTGATAGTCT |
| | | TTTTAAAGAGATTCAACGAATTCTCAACATTAAGTTGTA |
| | | AGGTTTTGATAGCATTCTGATTGAAATCAAATAACCTCA |
| | | TCGTATCGCAAAATTCTTCATTGTGATCTTTGTTGCATTT |
| | | TGCCATCACAGTGTTATCAAAACATTTTATTCCAGCCCA |
| | | AACAATAGCCCATTGCTCCAAACAGTAACCACCTGGGA |
| | | CATGTTGCCCAGTAGAGTCACTCAAGTCCCAAGTGAAA |
| | | AAGCCAAGGAGTTTCCTGCTCACAGAACTATAAGCAGT |
| | | TTTTTGGAGAGCCATCCTTATTGTTGCCATtGGAGTATAT |
| | | GTACAGTGATTTTCCCATGTGGTGTTCTGTATGATCAGG |
| | | AAATTGTAATGTGTCCCACCTTCACAGTTTGTTAGTCTG |
| | | CAAGACCCTCCACTACAGTTATTGAAACATTTTCCAACC |
| | | CACGCAATTTTTGGGTCCCCAATGATTTGAGCAAGCGAC |
| | | GCAATAAGATGTCTGCCAACCTCACCTCCTCTATCCCCA |
| | | ACTGTCAAGTTGTACTGGATCAACACCCCAGCACCCTCA |
| | | ACTGTTTTGCATCTGGCACCTACATGACGAGTGACATGG |
| | | AGCACATTGAAGTGTAACTCATTAAGCAACCATTTTAAT |
| | | GTGTGACCTGCTTCTTCTGTCTTATCACAATTACTAATGT |
| | | TACCATATGCAAGGCTTCTGATGTTGGAAAAGTTTCCAG |
| | | TAGTTTCATTTGCAATGGATGTGTTTGTCAAAGTGAGTT |
| | | CAATTCCCCATGTTGTGTTAGATGGTCCTTTGTAGTAAT |
| | | GATGTGTGTTGTTCTTGCTACATGATTGTGGCAAGTTGT |
| | | CAAACATTCTTGTGAGGTTGAACTCAACGTGGGTGAGAT |
| | | TGTGCCTCCTATCAATCATCATGCCATCACAACTTCTGC |
| | | CAGCCAAAATGAGGAAGGTGATGAGTTGGAATAGGCCA |
| | | CATCTCATCAGATTGACAAATCCTTTGATGATGCATAGG |
| | | GTTGAGACAATGATTAAGGCGACATTGAACACCTCCTG |
| | | CAGGACTTCGGGTATAGACTGGATCAAAGTCACAACTT |
| | | GTCCCATTTTGGGGTTGTTTGCACTTCCTCCGAGTCAGT |
| | | GAAGAAGTGAACGTACAGCGTGATCTAGAATCGCctaggat |
| | | ccactgtgcg |
| 18 | Amino acid | MIAAPLLLARAASLSLGFLFLLFFWLDRSVLAKELKFVTLV |
| | sequence of PAP | FRHGDRSPIDTFPTDPIKESSWPQGFGQLTQLGMEQHYELG |
| | with I2R mutation | EYIRKRYRKFLNESYKHEQVYIRSTDVDRTLMSAMTNLAA |
| | | LFPPEGVSIWNPILLWQPIPVHTVPLSEDQLLYLPFRNCPRF |
| | | QELESETLKSEEFQKRLHPYKDFIATLGKLSGLHGQDLFGI |
| | | WSKVYDPLYCESVHNFTLPSWATEDTMTKLRELSELSLLS |
| | | LYGIHKQKEKSRLQGGVLVNEILNHMKRATQIPSYKKLIM |
| | | YSAHDTTVSGLQMALDVYNGLLPPYASCHLTELYFEKGE |
| | | YFVEMYYRNETQHEPYPLMLPGCSPSCPLERFAELVGPVIP |
| | | QDWSTECMTTNSHQGTEDSTD |

What is claimed:

1. An arenavirus S segment, wherein the arenavirus S segment is engineered to carry an open reading frame (ORF) encoding a prostate cancer-related antigen or an antigenic fragment thereof, wherein the prostate cancer-related antigen is selected from the group consisting of: prostatic acid phosphatase (PAP), prostate specific antigen (PSA), and prostate-specific membrane antigen (PSMA).

2. The arenavirus S segment of claim 1, wherein the arenavirus S segment is engineered to carry a heterologous ORF encoding PAP or an antigenic fragment thereof in a position under control of an arenavirus 5′ UTR, and an ORF encoding arenaviral glycoprotein (GP) in a position under control of an arenavirus 3′ UTR.

3. The arenavirus S segment of claim 1, wherein the arenavirus S segment is engineered to carry a heterologous ORF encoding PAP or an antigenic fragment thereof in a position under control of an arenavirus 3′ UTR, and an ORF encoding GP in a position under control of an arenavirus 5′ UTR.

4. The arenavirus S segment of claim 1, wherein the arenavirus S segment is engineered to carry a heterologous ORF encoding PAP or an antigenic fragment thereof in a position under control of an arenavirus 5′ UTR, and an ORF encoding arenaviral nucleoprotein (NP) in a position under control of an arenavirus 3′ UTR.

5. The arenavirus S segment of claim 1, wherein the arenavirus S segment is engineered to carry a heterologous ORF encoding PAP or an antigenic fragment thereof in a position under control of an arenavirus 3′ UTR, and an ORF encoding NP in a position under control of an arenavirus 5′ UTR.

6. The arenavirus S segment of claim 1, wherein the arenavirus S segment is engineered to carry a heterologous ORF encoding PSA or an antigenic fragment thereof in a position under control of an arenavirus 5′ UTR, and an ORF encoding arenaviral glycoprotein (GP) in a position under control of an arenavirus 3′ UTR.

7. The arenavirus S segment of claim 1, wherein the arenavirus S segment is engineered to carry a heterologous ORF encoding PSA or an antigenic fragment thereof in a position under control of an arenavirus 3′ UTR, and an ORF encoding GP in a position under control of an arenavirus 5′ UTR.

8. The arenavirus S segment of claim 1, wherein the arenavirus S segment is engineered to carry a heterologous ORF encoding PSA or an antigenic fragment thereof in a position under control of an arenavirus 5′ UTR, and an ORF encoding arenaviral nucleoprotein (NP) in a position under control of an arenavirus 3′ UTR.

9. The arenavirus S segment of claim 1, wherein the arenavirus S segment is engineered to carry a heterologous ORF encoding PSA or an antigenic fragment thereof in a position under control of an arenavirus 3′ UTR, and an ORF encoding NP in a position under control of an arenavirus 5′ UTR.

10. The arenavirus S segment of claim 1, wherein the arenavirus S segment is engineered to carry a heterologous ORF encoding an antigenic fragment of PSMA in a position under control of an arenavirus 5′ UTR, and an ORF encoding arenaviral glycoprotein (GP) in a position under control of an arenavirus 3′ UTR.

11. The arenavirus S segment of claim 1, wherein the arenavirus S segment is engineered to carry a heterologous ORF encoding an antigenic fragment of PSMA in a position under control of an arenavirus 3′ UTR, and an ORF encoding GP in a position under control of an arenavirus 5′ UTR.

12. The arenavirus S segment of claim 1, wherein the arenavirus S segment is engineered to carry a heterologous ORF encoding an antigenic fragment of PSMA in a position under control of an arenavirus 5′ UTR, and an ORF encoding arenaviral nucleoprotein (NP) in a position under control of an arenavirus 3′ UTR.

13. The arenavirus S segment of claim 1, wherein the arenavirus S segment is engineered to carry a heterologous ORF encoding an antigenic fragment of PSMA in a position under control of an arenavirus 3′ UTR, and an ORF encoding NP in a position under control of an arenavirus 5′ UTR.

14. The arenavirus S segment of any one of claims 2 to 5, wherein the amino acid sequence of the PAP or an antigenic fragment thereof comprises at least 80% or 90% identity to SEQ ID NO: 1.

15. The arenavirus S segment of any one of claims 6 to 9, wherein the amino acid sequence of the PSA or an antigenic fragment thereof comprises at least 80% or 90% identity to SEQ ID NO: 2.

16. The arenavirus S segment of any one of claims 10 to 13, wherein the amino acid sequence of the antigenic fragment of PSMA comprises at least 80% or 90% identity to SEQ ID NO: 3 or SEQ ID NO: 4.

17. The arenavirus S segment of any one of claims 2 to 5, wherein the amino acid sequence of the PAP or an antigenic fragment thereof comprises SEQ ID NO: 1.

18. The arenavirus S segment of any one of claims 2 to 5, wherein the amino acid sequence of the PAP or an antigenic fragment thereof comprises SEQ ID NO: 18.

19. The arenavirus S segment of any one of claims 6 to 9, wherein the amino acid sequence of the PSA or an antigenic fragment thereof comprises SEQ ID NO: 2.

20. The arenavirus S segment of any one of claims 10 to 13, wherein the amino acid sequence of the antigenic fragment of PSMA comprises SEQ ID NO: 3 or SEQ ID NO: 4.

21. The arenavirus S segment of any one of claims 2 to 5, wherein the amino acid sequence of the PAP or an antigenic fragment thereof consists of SEQ ID NO: 1.

22. The arenavirus S segment of any one of claims 2 to 5, wherein the amino acid sequence of the PAP or an antigenic fragment thereof consists of SEQ ID NO: 18.

23. The arenavirus S segment of any one of claims 6 to 9, wherein the amino acid sequence of the PSA or an antigenic fragment thereof consists of SEQ ID NO: 2.

24. The arenavirus S segment of any one of claims 10 to 13, wherein the amino acid sequence of the antigenic fragment of PSMA consists of SEQ ID NO: 3 or SEQ ID NO: 4.

25. The arenavirus S segment of any one of claims 2 to 5, wherein the nucleotide sequence of the ORF encoding the PAP or an antigenic fragment thereof comprises at least 50%, 60%, 70%, 80%, or 90% identity to SEQ ID NO: 5.

26. The arenavirus S segment of any one of claims 6 to 9, wherein the nucleotide sequence of the ORF encoding the PSA or an antigenic fragment thereof comprises at least 50%, 60%, 70%, 80%, or 90% identity to SEQ ID NO: 6.

27. The arenavirus S segment of any one of claims 10 to 13, wherein the nucleotide sequence of the ORF encoding the antigenic fragment of PSMA comprises at least 50%, 60%, 70%, 80%, or 90% identity to SEQ ID NO: 7 or SEQ ID NO: 8.

28. The arenavirus S segment of any one of claims 2 to 5, wherein the nucleotide sequence of the ORF encoding the PAP or an antigenic fragment thereof comprises SEQ ID NO: 5.

29. The arenavirus S segment of any one of claims 6 to 9, wherein the nucleotide sequence of the ORF encoding the PSA or an antigenic fragment thereof comprises SEQ ID NO: 6.

30. The arenavirus S segment of any one of claims 10 to 13, wherein the nucleotide sequence of the ORF encoding the antigenic fragment of PSMA comprises SEQ ID NO: 7 or SEQ ID NO: 8.

31. The arenavirus S segment of any one of claims 2 to 5, wherein the nucleotide sequence of the ORF encoding the PAP or an antigenic fragment thereof consists of SEQ ID NO: 5.

32. The arenavirus S segment of any one of claims 6 to 9, wherein the nucleotide sequence of the ORF encoding the PSA or an antigenic fragment thereof consists of SEQ ID NO: 6.

33. The arenavirus S segment of any one of claims 10 to 13, wherein the nucleotide sequence of the ORF encoding the antigenic fragment of PSMA consists of SEQ ID NO: 7 or SEQ ID NO: 8.

34. A cDNA of the arenavirus S segment of any one of claims 1 to 33.

35. A DNA expression vector comprising the cDNA of claim 34.

36. A host cell comprising the arenavirus S segment of any one of claims 1 to 33, the cDNA of claim 34, or the vector of claim 35.

37. A tri-segmented arenavirus particle comprising one arenavirus L segment and two arenavirus S segments, wherein the two arenavirus S segments are any one of claims 1 to 33, and wherein one of the two arenavirus S segments comprises GP and the other comprises NP.

38. The tri-segmented arenavirus particle of claim 37, wherein the tri-segmented arenavirus particle has stable expression of the prostate cancer-related antigen or an antigenic fragment thereof after being passaged at least 4, 5, 6, 7, 8, 9, or 10 generations.

39. A tri-segmented arenavirus particle comprising one arenavirus L segment and two arenavirus S segments, wherein a first arenavirus S segment is engineered to carry a heterologous ORF consisting of SEQ ID NO: 5 in a position under control of an arenavirus 5′ UTR and an ORF encoding arenaviral nucleoprotein (NP) in a position under control of an arenavirus 3′ UTR, and a second arenavirus S segment is engineered to carry a heterologous ORF consisting of SEQ ID NO: 6 in a position under control of an arenavirus 5′ UTR and an ORF encoding arenaviral glycoprotein (GP) in a position under control of an arenavirus 3′ UTR.

40. A tri-segmented arenavirus particle comprising one arenavirus L segment and two arenavirus S segments, wherein a first arenavirus S segment is engineered to carry a heterologous ORF consisting of SEQ ID NO: 8 in a position under control of an arenavirus 5′ UTR and an ORF encoding arenaviral nucleoprotein (NP) in a position under control of an arenavirus 3′ UTR, and a second arenavirus S segment is engineered to carry a heterologous ORF consisting of SEQ ID NO: 7 in a position under control of an arenavirus 5′ UTR and an ORF encoding arenaviral glycoprotein (GP) in a position under control of an arenavirus 3′ UTR.

41. A tri-segmented arenavirus particle comprising one arenavirus L segment and two arenavirus S segments, wherein a first arenavirus S segment is engineered to carry a heterologous ORF consisting of SEQ ID NO: 7 in a position under control of an arenavirus 5′ UTR and an ORF encoding arenaviral nucleoprotein (NP) in a position under control of an arenavirus 3′ UTR, and a second arenavirus S segment is engineered to carry a heterologous ORF consisting of SEQ ID NO: 8 in a position under control of an arenavirus 5′ UTR and an ORF encoding arenaviral glycoprotein (GP) in a position under control of an arenavirus 3′ UTR.

42. The tri-segmented arenavirus particle of any one of claims 37 to 41, wherein the tri-segmented arenavirus particle is derived from lymphocytic choriomeningitis virus (LCMV) or Pichinde virus (PICV).

43. The tri-segmented arenavirus particle of claim 42, wherein the tri-segmented arenavirus particle is derived from LCMV.

44. The tri-segmented arenavirus particle of claim 43, wherein said LCMV is MP strain, WE strain, an LCMV clone 13 expressing the glycoprotein of LCMV strain WE instead of the endogenous LCMV clone 13 glycoprotein, Armstrong strain, or Armstrong Clone 13 strain.

45. The tri-segmented arenavirus particle claim 42, wherein the tri-segmented arenavirus particle is derived from PICV.

46. The tri-segmented arenavirus particle claim 45, wherein said PICV is strain Munchique CoAn4763 isolate P18, or P2 strain.

47. A tri-segmented arenavirus particle comprising two S segments, wherein one of the two S segments comprises SEQ ID NO. 10, and the other one of the two S segments comprises SEQ ID NO. 11.

48. A tri-segmented arenavirus particle comprising two S segments, wherein one of the two S segments comprises SEQ ID NO. 12, and the other one of the two S segments comprises SEQ ID NO. 13.

49. A tri-segmented arenavirus particle comprising two S segments, wherein one of the two S segments comprises SEQ ID NO. 14, and the other one of the two S segments comprises SEQ ID NO. 15.

50. A tri-segmented arenavirus particle comprising two S segments, wherein one of the two S segments comprises SEQ ID NO. 16, and the other one of the two S segments comprises SEQ ID NO. 17.

51. The tri-segmented arenavirus particle of any one of claims 37 to 50, wherein the tri-segmented arenavirus particle is infectious and replication competent.

52. The tri-segmented arenavirus particle of any one of claims 37 to 50, wherein the tri-segmented arenavirus particle is attenuated.

53. A method of generating a tri-segmented arenavirus particle, wherein the method comprises:
(i) transfecting into a host cell the nucleic acids of two arenavirus S segments and one arenavirus L segment, wherein the two arenavirus S segments are any one of claims 1 to 33;
(ii) maintaining the host cell under conditions suitable for virus formation; and
(iii) harvesting the cell culture supernatant containing the arenavirus particle.

54. The method of claim 53, wherein the transcription of the arenavirus L segment and the two arenavirus S segments is performed using a bidirectional expression cassette.

55. The method of claim 53 or 54, wherein the method further comprises transfecting one or more nucleic acids encoding an arenavirus polymerase into the host cell.

56. The method of claim 55, wherein the arenavirus polymerase is the arenavirus L protein.

57. The method of any one of claims 53 to 56, wherein the method further comprises transfecting one or more nucleic acids encoding the arenavirus NP protein into the host cell.

58. The method of any one of claims 53 to 57, wherein the nucleic acids are encoded in cDNA.

59. The method of any one of claims 53 to 57, wherein the nucleic acids are encoded in RNA.

60. The method of any one of claims 53 to 59, wherein transcription of the arenavirus L segment and the two arenavirus S segments are each under the control of a promoter.

61. The method of claim 58, wherein the promoter is selected from the group consisting of:
(i) a RNA polymerase I promoter;
(ii) a RNA polymerase II promoter; and
(iii) a T7 promoter.

62. A pharmaceutical composition comprising an arenavirus particle of any one of claims 37 to 52, and a pharmaceutically acceptable carrier.

63. A method for treating prostate cancer comprising administering to a subject in need thereof the pharmaceutical composition of claim 62 in a therapeutically effective amount.

64. A method for treating prostate cancer in a subject in need thereof, wherein the method comprises:
(i) administering to the subject a first pharmaceutical composition, wherein the first pharmaceutical composition comprises one or more arenavirus particles of any one of claims 37 to 52; and
(ii) administering to the subject, after a period of time, a second pharmaceutical composition, wherein the second pharmaceutical composition comprises one or more arenavirus particles of any one of claims 37 to 52.

65. The method of claim 64, wherein the method further comprises repeating (i) and (ii).

66. The method of claim 64 or 65, wherein the first and the second pharmaceutical compositions are administered intravenously or intratumorally.

67. The method of any one of claims 64 to 66, wherein the one or more arenavirus particles from the first pharmaceutical composition are derived from a different arenavirus species but carry ORF(s) encoding the same prostate cancer-related antigens or antigenic fragments thereof, when compared to the one or more arenavirus particles from the second pharmaceutical composition.

68. The method of claim 67, wherein the one or more arenavirus particles from the first pharmaceutical composition are derived from PICV, and the one or more arenavirus particles from the second pharmaceutical composition are derived from LCMV.

69. The method of any one of claims 64 to 68, wherein the one or more arenavirus particles from the first pharmaceutical composition is the tri-segmented arenavirus particle of claim 48, and the one or more arenavirus particles from the second pharmaceutical composition is the tri-segmented arenavirus particle of claim 47,

70. The method of any one of claims 64 to 68, wherein the one or more arenavirus particles from the first pharmaceutical composition are the tri-segmented arenavirus particles of claim 48 and claim 50, and the one or more arenavirus particles from the second pharmaceutical composition are the tri-segmented arenavirus particles of claim 47 and claim 49.

71. The method of any one of claims 64 to 70, wherein a second agent is administered in combination with the first and/or the second pharmaceutical composition.

72. The method of claim 71, wherein the second agent is an agent to treat prostate cancer.

73. The method of claim 72, wherein the second agent is selected from the group consisting of docetaxel, mitoxantrone, cabazitaxel, and pembrolizumab.

74. The method of claim 72, wherein the second agent is selected from the group consisting of enzalutamide and abiraterone.

75. The method of any one of claims 72 to 74, wherein the second agent is administered with a steroid.

76. The method of claim 75, wherein the steroid comprises prednisone or methylprednisolone.

77. The method of any one of claims 71 to 76, wherein the first and/or the second pharmaceutical composition and the second agent are co-administered simultaneously.

78. The method of any one of claims 71 to 76, wherein the first and/or the second pharmaceutical composition is administered prior to administration of the second agent.

79. The method of any one of claims 71 to 76, wherein the first and/or the second pharmaceutical composition is administered after administration of the second agent.

80. The method of any one of claims 78 to 79, wherein the interval between administration of the pharmaceutical composition(s) and the second agent is about 1 hour, about 2 hours, about 3 hours, about 4 hours, about 5 hours, about 6 hours, about 7 hours, about 8 hours, about 9 hours, about 10 hours, about 11 hours, about 12 hours, about 1 day, about 2 days, about 3 days, about 4 days, about 5 days, about 6 days, about 1 week, about 8 days, about 9 days, about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, or more.

81. The method of any one of claims 63 to 80, wherein the subject is suffering from, is susceptible to, or is at risk for prostate cancer.

82. A kit comprising a container and an instruction for use, wherein the container comprises an arenavirus particle of any one of claims 37 to 52, and optionally wherein the arenavirus particle is in a pharmaceutical composition suitable for intravenous administration.

83. A kit comprising two or more containers and an instruction for use, wherein one of the containers comprises an arenavirus particle of any one of claims 37 to 52, and another of the containers comprises a second agent, and optionally wherein the arenavirus particle is in a pharmaceutical composition suitable for intravenous administration.

84. The kit of any one of claims 82 to 83, wherein the kit further comprises an apparatus suitable for performing intravenous administration.


SEQUENCE LISTING (18 sequences)

SEQ ID NO: 1 - protein, 386 aa, Artificial Sequence
  Amino acid sequence of PAP
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 2 - protein, 261 aa, Artificial Sequence
  Amino acid sequence of PSA
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 3 - protein, 343 aa, Artificial Sequence
  Amino acid sequence of “PSMA1”
MWNLLHETDSAVATARRPRWLCAGALVLAGGFFLLGFLFGWFIKSSNEATNITPKHNMKAFLDELKAENIKKFLHNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVK

SEQ ID NO: 4 - protein, 407 aa, Artificial Sequence
  Amino acid sequence of “PSMA2”
MHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 5 - DNA, 1161 bp, Artificial Sequence
  Nucleotide sequence of PAP
atgagagctgcaccccttctcctggccagggcagcaagcctcagccttggcttcttgtttctgctttttttctggctggacagaagtgtgcttgccaaggagttgaagtttgtgactttggtgttcaggcatggagacagaagtcccattgacacctttcccacagaccccatcaaggaatcctcatggcctcaaggatttggtcaactcactcaactgggcatggagcagcactatgaacttggggagtacatcagaaagagatacagaaaattcttgaatgagtcctacaaacatgaacaggtttacatcagaagcacagatgttgacaggactttgatgagtgccatgacaaacctggcagccctgttcccccctgaaggtgtcagcatctggaatcccattctcctttggcaacccatcccagtgcacacagttcctctttctgaagatcagttgctctacctgcctttcaggaattgtccaaggtttcaagaacttgagagtgaaactttgaaatcagaagaatttcagaagaggctgcacccttacaaggatttcatagccaccttgggaaagctttcagggttgcatgggcaagacctttttggcatttggagcaaagtctatgaccctttatattgtgagagtgttcacaatttcaccttgccttcttgggccactgaggacaccatgacaaagttgagagaattgtcagaattgtccctcctgtctctctatggcattcacaagcagaaagagaaatccaggctccaagggggtgtcctggtcaatgaaatcctgaatcacatgaagagagcaactcagatcccaagctacaaaaaactcatcatgtattctgctcatgacacaactgtgagtggcctgcagatggctctagatgtttacaatggcctcctccctccctatgcttcttgccacttgacagaattgtattttgagaagggggagtactttgtggagatgtactacaggaatgagacccagcatgagccttatcctctcatgctgcctggctgcagccccagttgtcctcttgagagatttgctgagctggttggccctgtgatccctcaggactggtcaactgagtgcatgacaacaaacagtcatcaaggaactgaggacagcacagattag

SEQ ID NO: 6 - DNA, 786 bp, Artificial Sequence
  Nucleotide sequence of PSA
atgtgggtccctgtggtcttcctcaccctgtctgtgacttggattggagctgcccccctcatcctgtccaggattgtgggtggctgggagtgtgagaagcattcccaaccctggcaggtgctggtggcctccagaggcagggctgtgtgtgggggggtcctggtgcacccccagtgggtcctcactgctgcccactgcatcaggaacaagagtgtgatcttgctggggaggcacagcctgttccatcctgaagacacaggccaggtcttccaggtcagccacagcttcccccaccccctctatgacatgagcctcctgaagaacagattcctcaggcctggtgatgactccagccatgacctcatgctgctcaggctgtcagagcctgcagagctcactgatgctgtgaaggtcatggacctgcccacccaggagccagccctggggaccacctgctatgcctcaggctggggcagcattgaaccagaggagttcttgacccccaagaaacttcagtgtgtggacctccatgtcatctccaatgatgtgtgtgcccaagttcacccccagaaggtcaccaagttcatgctgtgtgctggaagatggacagggggcaaaagcacctgctctggtgactctgggggcccccttgtgtgcaatggtgtgctccaaggcatcacctcctggggcagtgagccatgtgccctgcctgaaaggccttccctgtacaccaaggtggttcattacaggaagtggatcaaggacacaattgtggccaacccctga

SEQ ID NO: 7 - DNA, 1032 bp, Artificial Sequence
  Nucleotide sequence of “PSMA1”
atgtggaatcttcttcatgaaactgactcagctgtggccacagccagaagacccaggtggctgtgtgcaggggcccttgttcttgcaggtggtttttttctccttggcttcctctttggttggttcatcaagtcttcaaatgaagcaaccaacatcactccaaagcacaacatgaaagcatttttggatgaattgaaagctgagaacatcaagaagtttttgcacaatttcacacagattccacatttggcaggaacagaacaaaactttcagcttgcaaagcaaattcaatcccagtggaaagaatttggtctggattctgttgagctggctcattatgatgttctgttgtcctacccaaacaagactcatcccaactacatctcaatcatcaatgaagatggaaatgagattttcaacacctctttgtttgaaccaccacctccaggatatgaaaatgtgtcagacattgttcctcctttcagtgctttttctcctcaaggcatgccagagggagatctggtctatgtcaactatgcaagaactgaagactttttcaaattggaaagagacatgaaaatcaattgctctgggaaaattgtcattgccagatatgggaaagttttcagaggcaacaaggtgaaaaatgcccagctggcaggtgccaaaggagtcattctctactctgaccctgcagactattttgctcctggggtgaaatcttatcctgatggttggaatcttcctggaggtggtgtccagaggggcaacatcctcaatctgaatggtgcaggagatccactcaccccaggttacccagcaaatgaatatgcttacagaagaggaattgcagaggctgttggtcttcccagcattcctgttcatccaattggatactatgatgcccagaaactcctggaaaagatgggtggttcagcacccccagacagcagctggagaggcagtctcaaagtgccatacaatgttggccctggtttcacaggaaacttttccactcaaaaagtcaaatga

SEQ ID NO: 8 - DNA, 1224 bp, Artificial Sequence
  Nucleotide sequence of “PSMA2”
atgcacatccattcaaccaatgaagtgacaagaatttacaatgtgattggaactctcagaggagcagtggaaccagacagatatgtcattctgggaggtcacagggactcctgggtgtttggtggaattgaccctcagagtggagcagctgtggttcatgaaattgtcaggagttttggaacactgaaaaaggaagggtggagacccagaagaacaattttgtttgcaagctgggatgcagaagaatttggtcttcttggttcaactgagtgggcagaggagaactcaagactccttcaggagagaggagtagcttacatcaatgctgactcatctattgaaggaaattacactctgagagttgattgcactccactaatgtacagcttggttcacaatctgacaaaagagctgaaaagccctgatgaagggtttgaaggaaaatctctttatgaaagttggacaaaaaaaagtccctccccagagttcagtggaatgcccaggatcagcaaattgggatctggaaatgattttgaggtgttcttccaaagacttggaattgcttcaggcagagcaaggtacaccaagaattgggaaaccaacaaattcagtggttatccactatatcacagtgtttatgaaacatatgagttggtggaaaagttttatgatccaatgttcaaatatcatctgactgtggcacaggtcagaggagggatggtgtttgagctggccaattccatagttctcccttttgattgcagagattatgctgtggttttgagaaagtatgctgacaaaatttacagcatttcaatgaaacatccacaggaaatgaagacatacagtgtctcatttgattcacttttttctgcagtgaagaatttcacagaaattgcttccaagttcagtgaaaggcttcaggactttgacaaaagcaacccaattgttttgagaatgatgaatgatcaactcatgtttctggaaagagcattcattgatcccttggggttgccagacaggcctttttacaggcatgtcatctatgccccaagcagtcacaacaagtatgcaggggagtcatttccaggaatttatgatgctctgtttgacattgaaagcaaagtggacccttccaaggcctggggagaagtgaagagacagatttatgttgcagccttcacagttcaggcagctgcagagactttgagtgaagttgcttaa

SEQ ID NO: 9 - DNA, 2253 bp, Artificial Sequence
  Nucleotide sequence of full PSMA
atgtggaatcttcttcatgaaactgactcagctgtggccacagccagaagacccaggtggctgtgtgcaggggcccttgttcttgcaggtggtttttttctccttggcttcctctttggttggttcatcaagtcttcaaatgaagcaaccaacatcactccaaagcacaacatgaaagcatttttggatgaattgaaagctgagaacatcaagaagtttttgcacaatttcacacagattccacatttggcaggaacagaacaaaactttcagcttgcaaagcaaattcaatcccagtggaaagaatttggtctggattctgttgagctggctcattatgatgttctgttgtcctacccaaacaagactcatcccaactacatctcaatcatcaatgaagatggaaatgagattttcaacacctctttgtttgaaccaccacctccaggatatgaaaatgtgtcagacattgttcctcctttcagtgctttttctcctcaaggcatgccagagggagatctggtctatgtcaactatgcaagaactgaagactttttcaaattggaaagagacatgaaaatcaattgctctgggaaaattgtcattgccagatatgggaaagttttcagaggcaacaaggtgaaaaatgcccagctggcaggtgccaaaggagtcattctctactctgaccctgcagactattttgctcctggggtgaaatcttatcctgatggttggaatcttcctggaggtggtgtccagaggggcaacatcctcaatctgaatggtgcaggagatccactcaccccaggttacccagcaaatgaatatgcttacagaagaggaattgcagaggctgttggtcttcccagcattcctgttcatccaattggatactatgatgcccagaaactcctggaaaagatgggtggttcagcacccccagacagcagctggagaggcagtctcaaagtgccatacaatgttggccctggtttcacaggaaacttttccactcaaaaagtcaaaatgcacatccattcaaccaatgaagtgacaagaatttacaatgtgattggaactctcagaggagcagtggaaccagacagatatgtcattctgggaggtcacagggactcctgggtgtttggtggaattgaccctcagagtggagcagctgtggttcatgaaattgtcaggagttttggaacactgaaaaaggaagggtggagacccagaagaacaattttgtttgcaagctgggatgcagaagaatttggtcttcttggttcaactgagtgggcagaggagaactcaagactccttcaggagagaggagtagcttacatcaatgctgactcatctattgaaggaaattacactctgagagttgattgcactccactaatgtacagcttggttcacaatctgacaaaagagctgaaaagccctgatgaagggtttgaaggaaaatctctttatgaaagttggacaaaaaaaagtccctccccagagttcagtggaatgcccaggatcagcaaattgggatctggaaatgattttgaggtgttcttccaaagacttggaattgcttcaggcagagcaaggtacaccaagaattgggaaaccaacaaattcagtggttatccactatatcacagtgtttatgaaacatatgagttggtggaaaagttttatgatccaatgttcaaatatcatctgactgtggcacaggtcagaggagggatggtgtttgagctggccaattccatagttctcccttttgattgcagagattatgctgtggttttgagaaagtatgctgacaaaatttacagcatttcaatgaaacatccacaggaaatgaagacatacagtgtctcatttgattcacttttttctgcagtgaagaatttcacagaaattgcttccaagttcagtgaaaggcttcaggactttgacaaaagcaacccaattgttttgagaatgatgaatgatcaactcatgtttctggaaagagcattcattgatcccttggggttgccagacaggcctttttacaggcatgtcatctatgccccaagcagtcacaacaagtatgcaggggagtcatttccaggaatttatgatgctctgtttgacattgaaagcaaagtggacccttccaaggcctggggagaagtgaagagacagatttatgttgcagccttcacagttcaggcagctgcagagactttgagtgaagttgcttaa

SEQ ID NO: 10 - DNA, 3041 bp, Artificial Sequence
  PAP-NP-S-segment of PMVS (12), which is a PMVS of artLCMV-PAP-NP/PSA-GP
gcgcaccggggatcctaggctttttggattgcgctttcctctagatcaactgggtgtcaggccctatcctacagaaggatgagagctgcaccccttctcctggccagggcagcaagcctcagccttggcttcttgtttctgctttttttctggctggacagaagtgtgcttgccaaggagttgaagtttgtgactttggtgttcaggcatggagacagaagtcccattgacacctttcccacagaccccatcaaggaatcctcatggcctcaaggatttggtcaactcactcaactgggcatggagcagcactatgaacttggggagtacatcagaaagagatacagaaaattcttgaatgagtcctacaaacatgaacaggtttacatcagaagcacagatgttgacaggactttgatgagtgccatgacaaacctggcagccctgttcccccctgaaggtgtcagcatctggaatcccattctcctttggcaacccatcccagtgcacacagttcctctttctgaagatcagttgctctacctgcctttcaggaattgtccaaggtttcaagaacttgagagtgaaactttgaaatcagaagaatttcagaagaggctgcacccttacaaggatttcatagccaccttgggaaagctttcagggttgcatgggcaagacctttttggcatttggagcaaagtctatgaccctttatattgtgagagtgttcacaatttcaccttgccttcttgggccactgaggacaccatgacaaagttgagagaattgtcagaattgtccctcctgtctctctatggcattcacaagcagaaagagaaatccaggctccaagggggtgtcctggtcaatgaaatcctgaatcacatgaagagagcaactcagatcccaagctacaaaaaactcatcatgtattctgctcatgacacaactgtgagtggcctgcagatggctctagatgtttacaatggcctcctccctccctatgcttcttgccacttgacagaattgtattttgagaagggggagtactttgtggagatgtactacaggaatgagacccagcatgagccttatcctctcatgctgcctggctgcagccccagttgtcctcttgagagatttgctgagctggttggccctgtgatccctcaggactggtcaactgagtgcatgacaacaaacagtcatcaaggaactgaggacagcacagattagagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttatcttcatactccctcgaagcttccctggtcatttcaacatcgataagcttaatgtccttcctattttgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgagttgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagagatcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

SEQ ID NO: 11 - DNA, 2486 bp, Artificial Sequence
  PSA-GP-S-segment of PMVS (12), which is a PMVS of artLCMV-PAP-NP/PSA-GP
gcgcaccggggatcctaggctttttggattgcgctttcctctagatcaactgggtgtcaggccctatcctacagaaggatgtgggtccctgtggtcttcctcaccctgtctgtgacttggattggagctgcccccctcatcctgtccaggattgtgggtggctgggagtgtgagaagcattcccaaccctggcaggtgctggtggcctccagaggcagggctgtgtgtgggggggtcctggtgcacccccagtgggtcctcactgctgcccactgcatcaggaacaagagtgtgatcttgctggggaggcacagcctgttccatcctgaagacacaggccaggtcttccaggtcagccacagcttcccccaccccctctatgacatgagcctcctgaagaacagattcctcaggcctggtgatgactccagccatgacctcatgctgctcaggctgtcagagcctgcagagctcactgatgctgtgaaggtcatggacctgcccacccaggagccagccctggggaccacctgctatgcctcaggctggggcagcattgaaccagaggagttcttgacccccaagaaacttcagtgtgtggacctccatgtcatctccaatgatgtgtgtgcccaagttcacccccagaaggtcaccaagttcatgctgtgtgctggaagatggacagggggcaaaagcacctgctctggtgactctgggggcccccttgtgtgcaatggtgtgctccaaggcatcacctcctggggcagtgagccatgtgccctgcctgaaaggccttccctgtacaccaaggtggttcattacaggaagtggatcaaggacacaattgtggccaacccctgaagaacagcgcctccctgactctccacctcgaaagaggtggagagtcagggaggcccagagggtctcagcgtcttttccagatagtttttacaccaggcaccttgaatgcaccacaactacagatccccttgttggtcaagcggtgtggctttggacatgaaccgccctttatgtgtctatgtgttggtatcttcacaagatgcagaaagatgctgattagatatgctgatgttgaaaacatcaaaagatccattaaggctaaaggagtactcccttgtctttttatgtagtccttcctcaacatctctgtgatcatgttatctgcttcttgttcgatttgatcactaaagtgggtctcattcaagtaggagccattagtgacaagccagcacttgggtacactagtctcaccagtcttagcatgttccagataccagaactttgagtaattacagtatggtacccccattagatctcttagatgattcctcatcaacagctgatcggaaatcagagaatttactgttgttttgaatacatgcaaggcagactctacatcttgcttgaacttactcagggcggccttgttgtaatcaattagtcgtagcatgtcacagaactcttcatcatgattgacattacattttgcaacagctgtattcccaaaacatttgagctctgcagcaaggatcatccatttggtcaggcaataaccacctggattttctactcctgaggagtctgacagggtccaggtgaatgtgcctgcaagtctcctagtgagaaactttgtcttttcctgagcaaagaggattctagacatcccaaaagggcctgcatatctacagtggttttcccaagtcctgttttgtatgattaggtactgatagcttgtttggctgcaccaagtggtcttgccatctgaacctgcccagccccagccacttctcatgtattttcctccaaaggcagttctaaacatgtccaagactctacctctgaaagtcctacactggcttatagcgctctgtgggtccgaaaatgacaagttgtattgaatggtgatgccattgttaaaatcacaagacactgctttgtggttggaattccctctaatactgaggtgcagactcgagactatactcatgagtgtatggtcaaaagtctttttgttgaaagcggaggttaagttgcaaaaattgtgattaaggatggagtcgttagtgaaagttagctccagtccagagcttcccatactgatgtagtgatgagagttgttggctgagcacgcattgggcatcgtcagatttaagtgagacatatcaaactccactgatttgaactggtaaacccctttatagatgtcgggaccattaaggccgtacatgccacaggacctaccagccaaaaaaaggaagctgaccagtgctaatatcccacaggtggcgaaattgtacacagctttgatgctcgtgattataatgagcacaataatgacaatgttgatgacctcatcaatgatgtgaggcaaagcctcaaacattgtcacaatctgacccatcttgttgctcaatggtttctcaagacaaatgcgcaatcaaatgcctaggatccactgtgcg

SEQ ID NO: 12 - DNA, 3056 bp, Artificial Sequence
  PAP-NP-S-segment of PMVS(05) cl32/05/05, which is a PMVS of artPICV-PAP-NP/PSA-GP
gcgcaccggggatcctaggcataccttggacgcgcatattacttgatcaaagatgatagctgcaccccttctcctggccagggcagcaagcctcagccttggcttcttgtttctgctttttttctggctggacagaagtgtgcttgccaaggagttgaagtttgtgactttggtgttcaggcatggagacagaagtcccattgacacctttcccacagaccccatcaaggaatcctcatggcctcaaggatttggtcaactcactcaactgggcatggagcagcactatgaacttggggagtacatcagaaagagatacagaaaattcttgaatgagtcctacaaacatgaacaggtttacatcagaagcacagatgttgacaggactttgatgagtgccatgacaaacctggcagccctgttcccccctgaaggtgtcagcatctggaatcccattctcctttggcaacccatcccagtgcacacagttcctctttctgaagatcagttgctctacctgcctttcaggaattgtccaaggtttcaagaacttgagagtgaaactttgaaatcagaagaatttcagaagaggctgcacccttacaaggatttcatagccaccttgggaaagctttcagggttgcatgggcaagacctttttggcatttggagcaaagtctatgaccctttatattgtgagagtgttcacaatttcaccttgccttcttgggccactgaggacaccatgacaaagttgagagaattgtcagaattgtccctcctgtctctctatggcattcacaagcagaaagagaaatccaggctccaagggggtgtcctggtcaatgaaatcctgaatcacatgaagagagcaactcagatcccaagctacaaaaaactcatcatgtattctgctcatgacacaactgtgagtggcctgcagatggctctagatgtttacaatggcctcctccctccctatgcttcttgccacttgacagaattgtattttgagaagggggagtactttgtggagatgtactacaggaatgagacccagcatgagccttatcctctcatgctgcctggctgcagccccagttgtcctcttgagagatttgctgagctggttggccctgtgatccctcaggactggtcaactgagtgcatgacaacaaacagtcatcaaggaactgaggacagcacagattaggccctagcctcgacatgggcctcgacgtcactccccaataggggagtgacgtcgaggcctctgaggacttgagctcagaggttgatcagatctgtgttgttcctgtacagcgtgtcaataggcaagcatctcatcggcttctggtccctaacccagcctgtcactgttgcatcaaacatgatggtatcaagcaatgcacagtgaggattcgcagtggtttgtgcagcccccttcttcttcttctttatgaccaaacctttatgtttggtgcagagtagattgtatctctcccagatctcatcctcaaaggtgcgtgcttgctcggcactgagtttcacgtcaagcacttttaagtctcttctcccatgcatttcgaacaaactgattatatcatctgaaccttgagcagtgaaaaccatgttttgaggtaaatgtctgatgattgaggaaatcaggcctggttgggcatcagccaagtcctttaaaaggagaccatgtgagtacttgctttgctctttgaaggacttctcatcgtggggaaatctgtaacaatgtatgtagttgcccgtgtcaggctggtagatggccatttccaccggatcatttggtgttccttcaatgtcaatccatgtggtagcttttgaatcaagcatctgaattgaggacacaacagtatcttctttctccttagggatttgtttaaggtccggtgatcctccgtttcttactggtggctggatagcactcggcttcgaatctaaatctacagtggtgttatcccaagccctcccttgaacttgagaccttgagccaatgtaaggccaaccatcccctgaaagacaaatcttgtatagtaaattttcataaggatttctctgtccgggtgtagtgctcacaaacataccttcacgattctttatttgcaatagactctttatgagagtactaaacatagaaggcttcacctggatggtctcaagcatattgccaccatcaatcatgcaagcagctgctttgactgctgcagacaaactgagattgtaccctgagatgtttatggctgatggctcattactaatgatttttagggcactgtgttgctgtgtgagtttctctagatctgtcatgttcgggaacttgacagtgtagagcaaaccaagtgcactcagcgcttggacaacatcattaagttgttcacccccttgctcagtcatacaagcgatggttaaggctggcattgatccaaattgattgatcaacaatgtattatccttgatgtcccagatcttcacaaccccatctctgttgcctgtgggtctagcattagcgaaccccattgagcgaaggatttcggctctttgttccaactgagtgtttgtgagattgcccccataaacaccaggctgagacaaactctcagttctagtgactttctttcttaacttgtccaaatcagatgcaagctccattagctcctctttggctaagcctcccaccttaagcacattgtccctctggattgatctcatattcatcagagcatcaacctctttgttcatgtctcttaacttggtcagatcagaatcagtccttttatctttgcgcatcattctttgaacttgagcaactttgtgaaagtcaagagcagataacagtgctcttgtgtccgacaacacatcagccttcacaggatgggtccagttggatagacccctcctaagggactgtacccagcggaatgatgggatgttgtcagacattttggggttgtttgcacttcctccgagtcagtgaagaagtgaacgtacagcgtgatctagaatcgcctaggatccactgtgcg

SEQ ID NO: 13 - DNA, 2522 bp, Artificial Sequence
  PSA-GP-S-segment of PMVS(05) cl32/05/05, which is a PMVS of artPICV-PAP-NP/PSA-GP
gcgcaccggggatcctaggcataccttggacgcgcatattacttgatcaaagatgtgggtccctgtggtcttcctcaccctgtctgtgacttggattggagctgcccccctcatcctgtccaggattgtgggtggctgggagtgtgagaagcattcccaaccctggcaggtgctggtggcctccagaggcagggctgtgtgtgggggggtcctggtgcacccccagtgggtcctcactgctgcccactgcatcaggaacaagagtgtgatcttgctggggaggcacagcctgttccatcctgaagacacaggccaggtcttccaggtcagccacagcttcccccaccccctctatgacatgagcctcctgaagaacagattcctcaggcctggtgatgactccagccatgacctcatgctgctcaggctgtcagagcctgcagagctcactgatgctgtgaaggtcatggacctgcccacccaggagccagccctggggaccacctgctatgcctcaggctggggcagcattgaaccagaggagttcttgacccccaagaaacttcagtgtgtggacctccatgtcatctccaatgatgtgtgtgcccaagttcacccccagaaggtcaccaagttcatgctgtgtgctggaagatggacagggggcaaaagcacctgctctggtgactctgggggtccccttgtgtgcaatggtgtgctccaaggcatcacctcctggggcagtgagccatgtgccctgcctgaaaggccttccctgtacaccaaggtggttcattacaggaagtggatcaaggacacaattgtggccaacccctgagccctagcctcgacatgggcctcgacgtcactccccaataggggagtgacgtcgaggcctctgaggacttgagcttatttacccagtctcacccatttgtagggtttctttgggattttataatacccacagctgcaaagagagttcctagtaatcctatgtggcttcggacagccatcaccaatgatgtgcctatgagtgggtattccaactaagtggagaaacactgtgatggtgtaaaacaccaaagaccagaagcaaatgtctgtcaatgctagtggagtcttaccttgtctttcttcatattcttttatcagcatttcattgtacagattctggctctcccacaaccaatcattcttaaaatgcgtttcattgaggtacgagccattgtgaactaaccaacactgcggtaaagaatgtctccctgtgatggtatcattgatgtaccaaaattttgtatagttgcaataagggattttggcaagctgtttgagactgtttctaatcacaagtgagtcagaaataagtccgttgatagtctttttaaagagattcaacgaattctcaacattaagttgtaaggttttgatagcattctgattgaaatcaaataacctcatcgtatcgcaaaattcttcattgtgatctttgttgcattttgccatcacagtgttatcaaaacattttattccagcccaaacaatagcccattgctccaaacagtaaccacctgggacatgttgcccagtagagtcactcaagtcccaagtgaaaaagccaaggagtttcctgctcacagaactataagcagttttttggagagccatccttattgttgccattggagtatatgtacagtgattttcccatgtggtgttctgtatgatcaggaaattgtaatgtgtcccaccttcacagtttgttagtctgcaagaccctccactacagttattgaaacattttccaacccacgcaatttttgggtccccaatgatttgagcaagcgacgcaataagatgtctgccaacctcacctcctctatccccaactgtcaagttgtactggatcaacaccccagcaccctcaactgttttgcatctggcacctacatgacgagtgacatggagcacattgaagtgtaactcattaagcaaccattttaatgtgtgacctgcttcttctgtcttatcacaattactaatgttaccatatgcaaggcttctgatgttggaaaagtttccagtagtttcatttgcaatggatgtgtttgtcaaagtgagttcaattccccatgttgtgttagatggtcctttgtagtaatgatgtgtgttgttcttgctacatgattgtggcaagttgtcaaacattcttgtgaggttgaactcaacgtgggtgagattgtgcctcctatcaatcatcatgccatcacaacttctgccagccaaaatgaggaaggtgatgagttggaataggccacatctcatcagattgacaaatcctttgatgatgcatagggttgagacaatgattaaggcgacattgaacacctcctgcaggacttcgggtatagactggatcaaagtcacaacttgtcccattttggggttgtttgcacttcctccgagtcagtgaagaagtgaacgtacagcgtgatctagaatcgcctaggatccactgtgcg

SEQ ID NO: 14 - DNA, 3104 bp, Artificial Sequence
  PSMA2-NP-S-segment of PMVS (09) cl 9/7/2, which is a PMVS of artLCMV-PSMA2-NP/PSMA1-GP
gcgcaccggggatcctaggctttttggattgcgctttcctctagatcaactgggtgtcaggccctatcctacagaaggatgcacatccattcaaccaatgaagtgacaagaatttacaatgtgattggaactctcagaggagcagtggaaccagacagatatgtcattctgggaggtcacagggactcctgggtgtttggtggaattgaccctcagagtggagcagctgtggttcatgaaattgtcaggagttttggaacactgaaaaaggaagggtggagacccagaagaacaattttgtttgcaagctgggatgcagaagaatttggtcttcttggttcaactgagtgggcagaggagaactcaagactccttcaggagagaggagtagcttacatcaatgctgactcatctattgaaggaaattacactctgagagttgattgcactccactaatgtacagcttggttcacaatctgacaaaagagctgaaaagccctgatgaagggtttgaaggaaaatctctttatgaaagttggacaaaaaaaagtccctccccagagttcagtggaatgcccaggatcagcaaattgggatctggaaatgattttgaggtgttcttccaaagacttggaattgcttcaggcagagcaaggtacaccaagaattgggaaaccaacaaattcagtggttatccactatatcacagtgtttatgaaacatatgagttggtggaaaagttttatgatccaatgttcaaatatcatctgactgtggcacaggtcagaggagggatggtgtttgagctggccaattccatagttctcccttttgattgcagagattatgctgtggttttgagaaagtatgctgacaaaatttacagcatttcaatgaaacatccacaggaaatgaagacatacagtgtctcatttgattcacttttttctgcagtgaagaatttcacagaaattgcttccaagttcagtgaaaggcttcaggactttgacaaaagcaacccaattgttttgagaatgatgaatgatcaactcatgtttctggaaagagcattcattgatcccttggggttgccagacaggcctttttacaggcatgtcatctatgccccaagcagtcacaacaagtatgcaggggagtcatttccaggaatttatgatgctctgtttgacattgaaagcaaagtggacctttccaaggcctggggagaagtgaagagacagatttatgttgcagccttcacagttcaggcagctgcagagactttgagtgaagttgcttaaagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttatcttcatactccctcgaagcttccctggtcatttcaacatcgataagcttaatgtccttcctattttgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgagttgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagagatcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

SEQ ID NO: 15 - DNA, 2732 bp, Artificial Sequence
  PSMA1-GP-S-segment of PMVS (09) cl 9/7/2, which is a PMVS of artLCMV-PSMA2-NP/PSMA1-GP
gcgcaccggggatcctaggctttttggattgcgctttcctctagatcaactgggtgtcaggccctatcctacagaaggatgtggaatcttcttcatgaaactgactcagctgtggccacagccagaagacccaggtggctgtgtgcaggggcccttgttcttgcaggtggtttttttctccttggcttcctctttggttggttcatcaagtcttcaaatgaagcaaccaacatcactccaaagcacaacatgaaagcatttttggatgaattgaaagctgagaacatcaagaagtttttgcacaatttcacacagattccacatttggcaggaacagaacaaaactttcagcttgcaaagcaaattcaatcccagtggaaagaatttggtctggattctgttgagctggctcattatgatgttctgttgtcctacccaaacaagactcatcccaactacatctcaatcatcaatgaagatggaaatgagattttcaacacctctttgtttgaaccaccacctccaggatatgaaaatgtgtcagacattgttcctcctttcagtgctttttctcctcaaggcatgccagagggagatctggtctatgtcaactatgcaagaactgaagactttttcaaattggaaagagacatgaaaatcaattgctctgggaaaattgtcattgccagatatgggaaagttttcagaggcaacaaggtgaaaaatgcccagctggcaggtgccaaaggagtcattctctactctgaccctgcagactattttgctcctggggtgaaatcttatcctgatggttggaatcttcctggaggtggtgtccagaggggcaacatcctcaatctgaatggtgcaggagatccactcaccccaggttacccagcaaatgaatatgcttacagaagaggaattgcagaggctgttggtcttcccagcattcctgttcatccaattggatactatgatgcccagaaactcctggaaaagatgggtggttcagcacccccagacagcagctggagaggcagtctcaaagtgccatacaatgttggccctggtttcacaggaaacttttccactcaaaaagtcaaatgaagaacagcgcctccctgactctccacctcgaaagaggtggagagtcagggaggcccagagggtctcagcgtcttttccagatagtttttacaccaggcaccttgaatgcaccacaactacagatccccttgttggtcaagcggtgtggctttggacatgaaccgccctttatgtgtctatgtgttggtatcttcacaagatgcagaaagatgctgattagatatgctgatgttgaaaacatcaaaagatccattaaggctaaaggagtactcccttgtctttttatgtagtccttcctcaacatctctgtgatcatgttatctgcttcttgttcgatttgatcactaaagtgggtctcattcaagtaggagccattagtgacaagccagcacttgggtacactagtctcaccagtcttagcatgttccagataccagaactttgagtaattacagtatggtacccccattagatctcttagatgattcctcatcaacagctgatcggaaatcagagaatttactgttgttttgaatacatgcaaggcagactctacatcttgcttgaacttactcagggcggccttgttgtaatcaattagtcgtagcatgtcacagaactcttcatcatgattgacattacattttgcaacagctgtattcccaaaacatttgagctctgcagcaaggatcatccatttggtcaggcaataaccacctggattttctactcctgaggagtctgacagggtccaggtgaatgtgcctgcaagtctcctagtgagaaactttgtcttttcctgagcaaagaggattctagacatcccaaaagggcctgcatatctacagtggttttcccaagtcctgttttgtatgattaggtactgatagcttgtttggctgcaccaagtggtcttgccatctgaacctgcccagccccagccacttctcatgtattttcctccaaaggcagttctaaacatgtccaagactctacctctgaaagtcctacactggcttatagcgctctgtgggtccgaaaatgacaagttgtattgaatggtgatgccattgttaaaatcacaagacactgctttgtggttggaattccctctaatactgaggtgcagactcgagactatactcatgagtgtatggtcaaaagtctttttgttgaaagcggaggttaagttgcaaaaattgtcattaagtatggagtcgttagtgaaagttagctccagtccagagcttcccatactgatgtagtgatgagagttgttggctgagcacgcattgggcatcgtcagatttaagtgagacatatcaaactccactgatttgaactggtaaacccctttatagatgtcgggaccattaaggccgtacatgccacaggacctaccagccaaaaaaaggaagctgaccagtgctaatatcccacaggtggcgaaattgtacacagctttgatgctcgtgattataatgagcacaataatgacaatgttgatgacctcatcaatgatgtgaggcaaagcctcaaacattgtcacaatctgacccatcttgttgctcaatggtttctcaagacaaatgcgcaatcaaatgcctaggatccactgtgcg

SEQ ID NO: 16 - DNA, 2927 bp, Artificial Sequence
  PSMA1-NP-S-segment of PMVS(26), which is a PMVS of artPICV-PSMA1-NP/PSMA2-GP
gcgcaccggggatcctaggcataccttggacgcgcatattacttgatcaaagatgtggaatcttcttcatgaaactgactcagctgtggccacagccagaagacccaggtggctgtgtgcaggggcccttgttcttgcaggtggtttttttctccttggcttcctctttggttggttcatcaagtcttcaaatgaagcaaccaacatcactccaaagcacaacatgaaagcatttttggatgaattgaaagctgagaacatcaagaagtttttgcacaatttcacacagattccacatttggcaggaacagaacaaaactttcagcttgcaaagcaaattcaatcccagtggaaagaatttggtctggattctgttgagctggctcattatgatgttctgttgtcctacccaaacaagactcatcccaactacatctcaatcatcaatgaagatggaaatgagattttcaacacctctttgtttgaaccaccacctccaggatatgaaaatgtgtcagacattgttcctcctttcagtgctttttctcctcaaggcatgccagagggagatctggtctatgtcaactatgcaagaactgaagactttttcaaattggaaagagacatgaaaatcaattgctctgggaaaattgtcattgccagatatgggaaagttttcagaggcaacaaggtgaaaaatgcccagctggcaggtgccaaaggagtcattctctactctgaccctgcagactattttgctcctggggtgaaatcttatcctgatggttggaatcttcctggaggtggtgtccagaggggcaacatcctcaatctgaatggtgcaggagatccactcaccccaggttacccagcaaatgaatatgcttacagaagaggaattgcagaggctgttggtcttcccagcattcctgttcatccaattggatactatgatgcccagaaactcctggaaaagatgggtggttcagcacccccagacagcagctggagaggcagtctcaaagtgccatacaatgttggccctggtttcacaggaaacttttccactcaaaaagtcaaatgagccctagcctcgacatgggcctcgacgtcactccccaataggggagtgacgtcgaggcctctgaggacttgagctcagaggttgatcagatctgtgttgttcctgtacagcgtgtcaataggcaagcatctcatcggcttctggtccctaacccagcctgtcactgttgcatcaaacatgatggtatcaagcaatgcacagtgaggattcgcagtggtttgtgcagcccccttcttcttcttctttatgaccaaacctttatgtttggtgcagagtagattgtatctctcccagatctcatcctcaaaggtgcgtgcttgctcggcactgagtttcacgtcaagcacttttaagtctcttctcccatgcatttcgaacaaactgattatatcatctgaaccttgagcagtgaaaaccatgttttgaggtaaatgtctgatgattgaggaaatcaggcctggttgggcatcagccaagtcctttaaaaggagaccatgtgagtacttgctttgctctttgaaggacttctcatcgtggggaaatctgtaacaatgtatgtagttgcccgtgtcaggctggtagatggccatttccaccggatcatttggtgttccttcaatgtcaatccatgtggtagcttttgaatcaagcatctgaattgaggacacaacagtatcttctttctccttagggatttgtttaaggtccggtgatcctccgtttcttactggtggctggatagcactcggcttcgaatctaaatctacagtggtgttatcccaagccctcccttgaacttgagaccttgagccaatgtaaggccaaccatcccctgaaagacaaatcttgtatagtaaattttcataaggatttctctgtccgggtgtagtgctcacaaacataccttcacgattctttatttgcaatagactctttatgagagtactaaacatagaaggcttcacctggatggtctcaagcatattgccaccatcaatcatgcaagcagctgctttgactgctgcagacaaactgagattgtaccctgagatgtttatggctgatggctcattactaatgatttttagggcactgtgttgctgtgtgagtttctctagatctgtcatgttcgggaacttgacagtgtagagcaaaccaagtgcactcagcgcttggacaacatcattaagttgttcacccccttgctcagtcatacaagcgatggttaaggctggcattgatccaaattgattgatcaacaatgtattatccttgatgtcccagatcttcacaaccccatctctgttgcctgtgggtctagcattagcgaaccccattgagcgaaggatttcggctctttgttccaactgagtgtttgtgagattgcccccataaacaccaggctgagacaaactctcagttctagtgactttctttcttaacttgtccaaatcagatgcaagctccattagctcctctttggctaagcctcccaccttaagcacattgtccctctggattgatctcatattcatcagagcatcaacctctttgttcatgtctcttaacttggtcagatcagaatcagtccttttatctttgcgcatcattctttgaacttgagcaactttgtgaaagtcaagagcagataacagtgctcttgtgtccgacaacacatcagccttcacaggatgggtccagttggatagacccctcctaagggactgtacccagcggaatgatgggatgttgtcagacattttggggttgtttgcacttcctccgagtcagtgaagaagtgaacgtacagcgtgatctagaatcgcctaggatccactgtgcg

SEQ ID NO: 17 - DNA, 2960 bp, Artificial Sequence
  PSMA2-GP-S-segment of PMVS(26), which is a PMVS of artPICV-PSMA1-NP/PSMA2-GP
gcgcaccggggatcctaggcataccttggacgcgcatattacttgatcaaagatgcacatccattcaaccaatgaagtgacaagaatttacaatgtgattggaactctcagaggagcagtggaaccagacagatatgtcattctgggaggtcacagggactcctgggtgtttggtggaattgaccctcagagtggagcagctgtggttcatgaaattgtcaggagttttggaacactgaaaaaggaagggtggagacccagaagaacaattttgtttgcaagctgggatgcagaagaatttggtcttcttggttcaactgagtgggcagaggagaactcaagactccttcaggagagaggagtagcttacatcaatgctgactcatctattgaaggaaattacactctgagagttgattgcactccactaatgtacagcttggttcacaatctgacaaaagagctgaaaagccctgatgaagggtttgaaggaaaatctctttatgaaagttggacaaaaaaaagtccctccccagagttcagtggaatgcccaggatcagcaaattgggatctggaaatgattttgaggtgttcttccaaagacttggaattgcttcaggcagagcaaggtacaccaagaattgggaaaccaacaaattcagtggttatccactatatcacagtgtttatgaaacatatgagttggtggaaaagttttatgatccaatgttcaaatatcatctgactgtggcacaggtcagaggagggatggtgtttgagctggccaattccatagttctcccttttgattgcagagattatgctgtggttttgagaaagtatgctgacaaaatttacagcatttcaatgaaacatccacaggaaatgaagacatacagtgtctcatttgattcacttttttctgcagtgaagaatttcacagaaattgcttccaagttcagtgaaaggcttcaggactttgacaaaagcaacccaattgttttgagaatgatgaatgatcaactcatgtttctggaaagagcattcattgatcccttggggttgccagacaggcctttttacaggcatgtcatctatgccccaagcagtcacaacaagtatgcaggggagtcatttccaggaatttatgatgctctgtttgacattgaaagcaaagtggacccttccaaggcctggggagaagtgaagagacagatttatgttgcagccttcacagttcaggcagctgcagagactttgagtgaagttgcttaagccctagcctcgacatgggcctcgacgtcactccccaataggggagtgacgtcgaggcctctgaggacttgagcttatttacccagtctcacccatttgtagggtttctttgggattttataatacccacagctgcaaagagagttcctagtaatcctatgtggcttcggacagccatcaccaatgatgtgcctatgagtgggtattccaactaagtggagaaacactgtgatggtgtaaaacaccaaagaccagaagcaaatgtctgtcaatgctagtggagtcttaccttgtctttcttcatattcttttatcagcatttcattgtacagattctggctctcccacaaccaatcattcttaaaatgcgtttcattgaggtacgagccattgtgaactaaccaacactgcggtaaagaatgtctccctgtgatggtatcattgatgtaccaaaattttgtatagttgcaataagggattttggcaagctgtttgagactgtttctaatcacaagtgagtcagaaataagtccgttgatagtctttttaaagagattcaacgaattctcaacattaagttgtaaggttttgatagcattctgattgaaatcaaataacctcatcgtatcgcaaaattcttcattgtgatctttgttgcattttgccatcacagtgttatcaaaacattttattccagcccaaacaatagcccattgctccaaacagtaaccacctgggacatgttgcccagtagagtcactcaagtcccaagtgaaaaagccaaggagtttcctgctcacagaactataagcagttttttggagagccatccttattgttgccattggagtatatgtacagtgattttcccatgtggtgttctgtatgatcaggaaattgtaatgtgtcccaccttcacagtttgttagtctgcaagaccctccactacagttattgaaacattttccaacccacgcaatttttgggtccccaatgatttgagcaagcgacgcaataagatgtctgccaacctcacctcctctatccccaactgtcaagttgtactggatcaacaccccagcaccctcaactgttttgcatctggcacctacatgacgagtgacatggagcacattgaagtgtaactcattaagcaaccattttaatgtgtgacctgcttcttctgtcttatcacaattactaatgttaccatatgcaaggcttctgatgttggaaaagtttccagtagtttcatttgcaatggatgtgtttgtcaaagtgagttcaattccccatgttgtgttagatggtcctttgtagtaatgatgtgtgttgttcttgctacatgattgtggcaagttgtcaaacattcttgtgaggttgaactcaacgtgggtgagattgtgcctcctatcaatcatcatgccatcacaacttctgccagccaaaatgaggaaggtgatgagttggaataggccacatctcatcagattgacaaatcctttgatgatgcatagggttgagacaatgattaaggcgacattgaacacctcctgcaggacttcgggtatagactggatcaaagtcacaacttgtcccattttggggttgtttgcacttcctccgagtcagtgaagaagtgaacgtacagcgtgatctagaatcgcctaggatccactgtgcg

SEQ ID NO: 18 - protein, 386 aa, Artificial Sequence
  Amino acid sequence of PAP with I2R mutation
MIAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD